(19) United States
(12) Patent Application Publication
(10) Pub. No.: US 2026/0265797 A1
(43) Pub. Date: Sep. 10, 2026

(54) CONVERSION OF HOMOFARNESOL TO AMBROXIDE

(71) Applicant: Conagen Inc., Bedford, MA (US)

(72) Inventors: Cody Ryan Lemke, Somerville, MA (US); Yisheng Wu, Bedford, MA (US)

(73) Assignee: Conagen Inc., Bedford, MA (US)

(21) Appl. No.: 19/218,162

(22) Filed: May 23, 2025

Related U.S. Application Data
(63) Continuation of application No. PCT/US23/80885, filed on Nov. 22, 2023.
(60) Provisional application No. 63/427,522, filed on Nov. 23, 2022.

(51) Int. Cl. C12P 17/04; C12N 9/90

(52) U.S. Cl. CPC C12P 17/04; C12N 9/90; C12Y 504/99017

(57) ABSTRACT

The present disclosure relates, at least in part, to the production of ambroxide or a derivative thereof from homofarnesol. The production can be mediated in a cellular system (e.g., an Escherichia coli bacterium) or in an enzymatic reaction mixture without a cellular system.

### RELATED APPLICATION

This application is a Continuation of International Patent Application Serial No. PCT/US23/80885, filed Nov. 22, 2023, entitled “CONVERSION OF HOMOFARNESOL TO AMBROXIDE”, which is an application that claims the benefit under 35 U.S.C. § 119(e) to U.S. Provisional Application No. 63/427,522 filed on Nov. 23, 2022, entitled “CONVERSION OF HOMOFARNESOL TO AMBROXIDE”, the entire contents of each of which are incorporated herein by reference.

### REFERENCE TO AN ELECTRONIC SEQUENCE LISTING

The contents of the electronic sequence listing (C149770075US01-SUBSEQ-JAV.xml; Size: 108,554 bytes; and Date of Creation: May 21, 2026) is herein incorporated by reference in its entirety.

### FIELD OF THE INVENTION

The present disclosure generally relates to methods and materials for the conversion of homofarnesol to ambroxide in bacteria, yeasts or other cellular systems, or in non-cell systems. The present disclosure relates to the discovery of several enzymes, and variants thereof, capable of converting homofarnesol to ambroxide as well as to their nucleotide and protein sequences.

### SUMMARY

Ambroxide is one of the key components of ambergris which is a solid, waxy, flammable substance of a dull gray or blackish color produced in the digestive system of sperm whales. Considering the value of this compound within the perfume industry and the unsustainable nature of its production, a sustainable and alternative biosynthetic approach to producing this compound is of interest. Here, several enzymes capable of catalyzing a reaction to form ambroxide using homofarnesol as a substrate were identified. These enzymes include AghSHC1, AteSHC1, ApaSHC1, PfuSHC1, KdiSHC1, AorSHC1, KrhSHC1, AmaSHC1, RacSHC1, AmeSHC1, AdsSHC1, RfaSHC1, KmeSHC1, MflSHC1, AsySHC1, BstSHC1 and GfrSHC2 and were isolated from various bacterial species and were screened using whole cell bioconversion assays including homofarnesol substrate. Circular permutants were also made and were shown to be functional homofarnesol-ambroxide cyclases, some of which exhibited improved performance of the enzyme.

Ambroxide produced using the methods, enzymes and/or the isolated recombinant host cells or lysates described herein can be purified and incorporated into a consumer product. For example, the ambroxide can be admixed with a consumer product. In some embodiments, the ambroxide can be incorporated into the consumer product in an amount sufficient to impart, modify, boost or enhance a desirable fragrance, scent or odor, or to conceal, modify, or minimize an undesirable fragrance, scent or odor, in the consumer product. The consumer product, for example, can be selected from the group consisting of fragrances, cosmetics, toiletries, home and body care, detergents, repellents, fertilizers, air fresheners, and soaps.

Provided herein in one aspect is a method of producing ambroxide with any one of the enzymes provided herein, including a variant thereof. In another aspect, compositions comprising any one of the enzymes provided herein, including a variant thereof, is provided. In still another aspect, methods of producing any one of the enzymes provided herein, including a variant thereof, is provided. In yet another aspect, nucleic acids and polypeptides encoding any one of the enzymes, including a variant thereof, is provided. In still another aspect, a host cell comprising any one of the nucleic acids provided herein or that produces any one of the enzymes, including a variant thereof, or polypeptide provided herein is provided. In another aspect, compositions comprising ambroxide produced in any one of the methods provided herein or using any one of the enzymes, including a variant thereof, provided herein is provided.

Other features and advantages of the present disclosure will become apparent in the following detailed description, taken with reference to the accompanying drawings.

### BRIEF DESCRIPTION OF THE DRAWINGS

FIG. 1 shows a plasmid map of an exemplary expression vector used for the expression of homofarnesol-ambroxide cyclases (e.g., squalene-hopene cyclases).

FIG. 2 shows sequence homology among modified squalene-hopene cyclases (SHC) (AghSHC1:F640Y and circular permutation variants AghSHC1:CP3/Linker12/F640Y and AghSHC1:CP6/Linker12/F640Y) to other patented squalene-hopene cyclases. GmoSHC1 (International Flavors and Fragrances), AacSHC1 (Givaudan), ZmoSHC1 (BASF), BjaSHC1 (BASF), RpaSHC1 (BASF). Needle (EMBOSS) was used to generate the percent identities using pairwise alignments.

FIG. 3 shows a performance comparison of modified homofarnesol-ambroxide cyclases (e.g., squalene-hopene cyclases (SHC)) (AghSHC1:F640Y and circular permutation variants AghSHC1:CP3/Linker12/F640Y and AghSHC1:CP6/Linker12/F640Y) to other patented squalene-hopene cyclases. GmoSHC1 (International Flavors and Fragrances), AacSHC1:215G2 (Givaudan), ZmoSHC1 (BASF). Bioconversion was performed with 25 g/L homofarnesol substrate.

FIG. 4 shows a performance comparison of functionally characterized homofarnesol-ambroxide cyclases (e.g., squalene hopene cyclases) that were isolated in the efforts outlined in this patent. Bioconversion was performed with 10 g/L homofarnesol substrate.

FIG. 5 shows sequence homology between AghSHC1 and the rest of the squalene hopene cyclases functionally characterized herein. Needle (EMBOSS) was used to generate the percent identities using pairwise alignments.

FIG. 6 shows a structure model of AghSHC1 (SEQ ID NO: 3) which displays the locations of the selected cut sites for circular permutation. The structure was modelled using SWISS-MODEL and 2SQC as a template.

### DETAILED DESCRIPTION

As used herein, the singular forms “a,” “an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean serving as an example, instance, or illustration. Any embodiment described herein as “exemplary” is not necessarily to be construed as preferred or advantageous over other embodiments.

“Cellular system” is any cells that provide for the expression of proteins. It includes bacteria, yeast, plant cells and animal cells. It includes both prokaryotic and eukaryotic cells. It also includes the in vitro expression of proteins based on cellular components, such as ribosomes.

“Coding sequence” is to be given its ordinary and customary meaning to a person of ordinary skill in the art and is used without limitation to refer to a DNA sequence that encodes a specific amino acid sequence.

“Growing” or “cultivating” a cellular system includes providing an appropriate medium that would allow cells to multiply and divide. It also includes providing resources so that cells or cellular components can translate and make recombinant proteins.

“Yeasts” are eukaryotic, single-celled microorganisms classified as members of the fungus kingdom. Yeasts are unicellular organisms which evolved from multicellular ancestors but with some species useful for the current disclosure being those that have the ability to develop multicellular characteristics by forming strings of connected budding cells known as pseudo hyphae or false hyphae.

The term “complementary” is to be given its ordinary and customary meaning to a person of ordinary skill in the art and is used without limitation to describe the relationship between nucleotide bases that are capable to hybridizing to one another. For example, with respect to DNA, adenosine is complementary to thymine and cytosine is complementary to guanine. Accordingly, the subjection technology also includes isolated nucleic acid fragments that are complementary to the complete sequences as reported in the accompanying Sequence Listing as well as those substantially similar nucleic acid sequences.

The terms “nucleic acid” and “nucleotide” are to be given their respective ordinary and customary meanings to a person of ordinary skill in the art and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. In any one embodiments provided herein, a particular nucleic acid sequence can also encompass conservatively modified or degenerate variants thereof (e.g., degenerate codon substitutions) and complementary sequences, as well as the sequence explicitly indicated.

The term “isolated” is to be given its ordinary and customary meaning to a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “incubating” and “incubation” as used herein means a process of mixing two or more chemical or biological entities (such as a chemical compound and an enzyme) and allowing them to interact under conditions favorable for producing ambroxide.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxyinosine residues. A nucleic acid sequence and all of its degenerate variants will express the same amino acid or polypeptide.

The terms “polypeptide,” “protein,” and “peptide” are to be given their respective ordinary and customary meanings to a person of ordinary skill in the art; the three terms are sometimes used interchangeably and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a polynucleotide product. Thus, exemplary polypeptides include polynucleotide products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full-length polypeptide or protein, and has substantially the same biological activity, or carries out substantially the same function as the full-length polypeptide or protein (e.g., carrying out the same enzymatic reaction). In any one embodiment, the polypeptide may be a functional fragment of AghSHC1.

A functional fragment or domain of the polypeptides provided herein may be combined with one or more other functional fragments or domains to produce a chimeric polypeptide. “Chimeric polypeptides” as used herein refer to a polypeptide that comprises at least one polypeptide as provided herein, or a functional fragment or domain thereof, with another polypeptide, or functional fragment or domain thereof. The chimeric polypeptide may be a circular permutant. The circular permutant can be obtained by shuffling protein domains or homologs thereof.

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In an aspect, a variant is a “functional variant” which retains some or all of the ability of the reference polypeptide. In any one embodiment, the polypeptide may be a functional variant of AghSHC1.

The term “functional variant” further includes conservatively substituted variants. The term “conservatively substituted variant” refers to a peptide having an amino acid sequence that differs from a reference peptide by one or more conservative amino acid substitutions and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide. In any one embodiment, the polypeptide may be a variant of AghSHC1 with any one of the foregoing percentage identities. Preferably such a AghSHC1 polypeptide is functional in the conversion of homofarnesol to ambroxide.

The term “homologous” in all its grammatical forms and spelling variations refers to the relationship between polynucleotides or polypeptides that possess a “common evolutionary origin,” including polynucleotides or polypeptides from super families and homologous polynucleotides or proteins from different species (Reeck et al., CELL 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 900 at least 91%, at least 92%, at least 93%, at least 94%, at least 95%, at least 96%, at least 97%, at least 98%, at least 99%, and even 100% identical.

“Suitable regulatory sequences” is to be given its ordinary and customary meaning to a person of ordinary skill in the art and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art and is used without limitation to refer to a DNA sequence capable of controlling the expression of a coding sequence or functional RNA. In general, a coding sequence is located 3′ to a promoter sequence. Promoters may be derived in their entirety from a native gene or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters, which cause a gene to be expressed in most cell types at most times, are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” as used herein, is to be given its ordinary and customary meaning to a person of ordinary skill in the art and is used without limitation to refer to the transcription and stable accumulation of sense (mRNA) or antisense RNA derived from the nucleic acid fragment of the subject technology or production of a gene product in transgenic, transformed or recombinant organisms.

“Transformation” is to be given its ordinary and customary meaning to a person of ordinary skill in the art and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosomal. Host organisms containing the transformed nucleic acid fragments are referred to as “transgenic” or “transformed” or “recombinant”.

The terms “transformed,” “transgenic,” and “recombinant,” when used herein in connection with host cells, are to be given their respective ordinary and customary meanings to a person of ordinary skill in the art and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are to be given their ordinary and customary meanings to a person of ordinary skill in the art and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

“Protein Expression” refers to protein production that occurs after gene expression. It consists of the stages after DNA has been transcribed to messenger RNA (mRNA). The mRNA is then translated into polypeptide chains, which are ultimately folded into proteins. DNA is present in the cells through transfection—a process of deliberately introducing nucleic acids into cells. The term is often used for non-viral methods in eukaryotic cells. It may also refer to other methods and cell types, although other terms are preferred: “transformation” is more often used to describe non-viral DNA transfer in bacteria, non-animal eukaryotic cells, including plant cells. In animal cells, transfection is the preferred term as transformation is also used to refer to progression to a cancerous state (carcinogenesis) in these cells. Transduction is often used to describe virus-mediated DNA transfer. Transformation, transduction, and viral infection are included under the definition of transfection for this application.

The terms “plasmid,” “vector,” and “cassette” are to be given their respective ordinary and customary meanings to a person of ordinary skill in the art and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc., Burlington, MA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this disclosure “percent identity” may also be determined using BLASTX version 2.0 for translated nucleotide sequences and BLASTN version 2.0 for polynucleotide sequences.

The percent of sequence identity is preferably determined using the “Best Fit” or “Gap” program of the Sequence Analysis Software Package™ (Version 10; Genetics Computer Group, Inc., Madison, WI). “Gap” utilizes the algorithm of Needleman and Wunsch (Needleman and Wunsch, JOURNAL OF MOLECULAR BIOLOGY 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, ADVANCES IN APPLIED MATHEMATICS, 2:482-489, 1981, Smith et al., NUCLEIC ACIDS RESEARCH 11:2205-2220, 1983). The percent identity is most preferably determined using the “Best Fit” program.

Useful methods for determining sequence identity are also disclosed in the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. MOL. BIOL. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, the term “substantial percent sequence identity” refers to a percent sequence identity of at least about 70% sequence identity, at least about 80% sequence identity, at least about 85% identity, at least about 90% sequence identity, or even greater sequence identity, such as about 98% or about 99% sequence identity. Thus, one embodiment of the disclosure is a polynucleotide molecule that has at least about 70% sequence identity, at least about 80% sequence identity, at least about 85% identity, at least about 90% sequence identity, or even greater sequence identity, such as about 98% or about 99% sequence identity with a polynucleotide sequence described herein. Polynucleotide molecules that have the activity genes of the current disclosure are useful in the production of ambroxide as provided herein and have a substantial percent sequence identity to the polynucleotide sequences provided herein and are encompassed within the scope of this disclosure.

Identity is the fraction of amino acids that are the same between a pair of sequences after an alignment of the sequences (which can be done using only sequence information or structural information or some other information, but usually it is based on sequence information alone), and similarity is the score assigned based on an alignment using some similarity matrix. The similarity index can be any one of the following BLOSUM62, PAM250, or GONNET, or any matrix used by one skilled in the art for the sequence alignment of proteins.

Identity is the degree of correspondence between two sub-sequences (no gaps between the sequences). An identity of 25% or higher implies similarity of function, while 18-25% implies similarity of structure or function. Keep in mind that two completely unrelated or random sequences (that are greater than 100 residues) can have higher than 20% identity. Similarity is the degree of resemblance between two sequences when they are compared. This is dependent on their identity.

### Coding Nucleic Acid Sequences

The present disclosure relates to nucleic acid sequences that code for a polypeptide as described herein and which, in some embodiments, can be applied to perform the required genetic engineering manipulations. The present disclosure also relates to nucleic acids with a certain degree of “identity” to the sequences specifically disclosed herein. For example, aspects of the present disclosure encompass a nucleic acid sequence with at least 60% identity to the nucleotide sequence that encodes the polypeptide of any one of SEQ ID NOs: 2, 36, and 38, at least 65% identity to the nucleotide sequence that encodes the polypeptide of any one of SEQ ID NOs: 2, 36, and 38, at least 70% identity to the nucleotide sequence that encodes the polypeptide of any one of SEQ ID NOs: 2, 36, and 38, at least 75% identity to the nucleotide sequence that encodes the polypeptide of any one of SEQ ID NOs: 2, 36, and 38, at least 80% identity to the nucleotide sequence that encodes the polypeptide of any one of SEQ ID NOs: 2, 36, and 38, at least 85% identity to the nucleotide sequence that encodes the polypeptide of any one of SEQ ID NOs: 2, 36, and 38, at least 90% identity to the nucleotide sequence that encodes the polypeptide of any one of SEQ ID NOs: 2, 36, and 38, at least 95% identity to the nucleotide sequence that encodes the polypeptide of any one of SEQ ID NOs: 2, 36, and 38, or at least 97%, 98% or 99% identity to the nucleotide sequence that encodes the polypeptide of any one of SEQ ID NOs: 2, 36, and 38, In some embodiments, the nucleic acid sequence used to encode an polypeptide useful for the present disclosure can have a nucleic acid sequence identical to any one of SEQ ID NOs: 2, 36, and 38.

The present disclosure also relates to nucleic acid sequences coding for a polypeptide that has an amino acid sequence with at least 60% identity to the polypeptide of any one of SEQ ID NOs: 3, 37, or 39, at least 65% identity to the polypeptide of any one of SEQ ID NOs: 3, 37, or 39, at least 70% identity to the polypeptide of any one of SEQ ID NOs: 3, 37, or 39, at least 75% identity to the polypeptide of any one of SEQ ID NOs: 3, 37, or 39, at least 80% identity to the polypeptide of any one of SEQ ID NOs: 3, 37, or 39, at least 85% identity to the polypeptide of any one of SEQ ID NOs: 3, 37, or 39, at least 90% identity to the polypeptide of any one of SEQ ID NOs: 3, 37, or 39, at least 95% identity to the polypeptide of any one of SEQ ID NOs: 3, 37, or 39, or at least 97%, 98% or 99% identity to the polypeptide of any one of SEQ ID NOs: 3, 37, or 39, In some embodiments, the polypeptide can have an amino acid sequence identical to the polypeptide of any one of SEQ ID NOs: 3, 37, or 39, In some embodiments, the present disclosure can relate to nucleic acid sequences coding for any of the foregoing or a functional equivalent of any of the foregoing.

### Constructs According to the Present Disclosure

In some aspects, the present disclosure relates to constructs like expression vectors for expressing a polypeptide provided herein, such as an AghSHC1 polypeptide, which includes variants thereof.

In an embodiment, the expression vector includes those genetic elements for expression of the recombinant polypeptide described herein (i.e., CfIEM) in various host cells. The elements for transcription and translation in the host cell can include a promoter, a coding region for the protein complex, and a transcriptional terminator.

A person of ordinary skill in the art will be aware of the molecular biology techniques available for the preparation of expression vectors. The polynucleotide used for incorporation into the expression vector of the subject technology, as described above, can be prepared by routine techniques such as polymerase chain reaction (PCR). In molecular cloning, a vector is a DNA molecule used as a vehicle to artificially carry foreign genetic material into another cell, where it can be replicated and/or expressed (e.g. plasmid, cosmid, Lambda phages). A vector containing foreign DNA is considered recombinant DNA. The four major types of vectors are plasmids, viral vectors, cosmids, and artificial chromosomes. Of these, the most commonly used vectors are plasmids. Common to all engineered vectors are an origin of replication, a multicloning site, and a selectable marker.

A number of molecular biology techniques have been developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In an alternative embodiment, synthetic linkers containing one or more restriction sites provide are used to operably link the polynucleotide of the subject technology to the expression vector. In an embodiment, the polynucleotide is generated by restriction endonuclease digestion. In an embodiment, the nucleic acid molecule is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerizing activities, thereby generating blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector having ligation-independent cloning (LIC) sites can be employed. The required PCR amplified polynucleotide can then be cloned into the LIC vector without restriction digest or ligation (Aslanidis and de Jong, NUCL. ACID. RES. 18 6069-74, (1990), Haun et al, BIOTECHNIQUES 13, 515-18 (1992), each of which are incorporated herein by reference).

In an embodiment, in order to isolate and/or modify the polynucleotide of interest for insertion into the chosen plasmid, it is suitable to use PCR. Appropriate primers for use in PCR preparation of the sequence can be designed to isolate the required coding region of the nucleic acid molecule, add restriction endonuclease or LIC sites, place the coding region in the desired reading frame.

In an embodiment, a polynucleotide for incorporation into an expression vector of the subject technology is prepared using PCR appropriate oligonucleotide primers. The coding region is amplified, whilst the primers themselves become incorporated into the amplified sequence product. In an embodiment, the amplification primers contain restriction endonuclease recognition sites, which allow the amplified sequence product to be cloned into an appropriate vector.

The expression vectors can be introduced into plant or microbial host cells by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Successfully transformed cells, that is, those cells containing the expression vector, can be identified by techniques well known in the art. For example, cells transfected with an expression vector of the subject technology can be cultured to produce polypeptides described herein. Cells can be examined for the presence of the expression vector DNA by techniques well known in the art.

The host cells can contain a single copy of the expression vector described previously, or alternatively, multiple copies of the expression vector.

In some embodiments, the transformed cell is a plant cell, an algal cell, a fungal cell that is not Colletotrichum, or a yeast cell. In some embodiments, the cell is a plant cell selected from the group consisting of: canola plant cell, a rapeseed plant cell, a palm plant cell, a sunflower plant cell, a cotton plant cell, a corn plant cell, a peanut plant cell, a flax plant cell, a sesame plant cell, a soybean plant cell, and a petunia plant cell.

Microbial host cell expression systems and expression vectors containing regulatory sequences that direct high-level expression of foreign proteins that are well-known to those skilled in the art. Any of these could be used to construct vectors for expression of the recombinant polypeptide of the subjection technology in a microbial host cell. These vectors could then be introduced into appropriate microorganisms via transformation to allow for high level expression of the recombinant polypeptide of the subject technology.

Vectors or cassettes useful for the transformation of suitable microbial host cells are well known in the art. Typically the vector or cassette contains sequences directing transcription and translation of the relevant polynucleotide, a selectable marker, and sequences allowing autonomous replication or chromosomal integration. Suitable vectors comprise a region 5′ of the polynucleotide which harbors transcriptional initiation controls and a region 3′ of the DNA fragment which controls transcriptional termination. It is preferred for both control regions to be derived from genes homologous to the transformed host cell, although it is to be understood that such control regions need not be derived from the genes native to the specific species chosen as a host.

Termination control regions may also be derived from various genes native to the microbial hosts. A termination site optionally may be included for the microbial hosts described herein.

Preferred host cells include those known to have the ability to produce ambroxide from homfarnesol. For example, preferred host cells can include bacteria of the genus Escherichia.

### Polypeptides

The polypeptides provided herein include those with functional fragments and/or the percent identities provided above. The polypeptides provided herein, such as the AghSHC1 polypeptides, including circular permutants thereof, have been found to exhibit high activity. Thus, the polypeptides may be in the form of a circular permutant. The circular permutants of AghSHC1 polypeptides, which polypeptides include those with a functional fragment and/or the percent identities provided above and retain desirable activity to that of SEQ ID NO: 3, may be made using any one or more of the cut sites provided herein, such as Cut Site 3 and Cut Site 6.

In addition, in one embodiment, a linker may be used in the circular permutant. The linker may be 6-12 amino acids in length, in one embodiment. The linker may be any one of the linkers provided herein. In one embodiment, the linker is Linker12.

### Production of Ambroxide

The inventors have surprisingly discovered that certain polypeptides have surprisingly high activity in the bioconversion of homofarnesol to ambroxide compared to other enzymes. More specifically, by engineering a host cell as provided herein and cultivating the engineered host strain in a mixture including homofarnesol, the inventors were able to achieve high levels of ambroxide production.

Cultivation of host cells can be carried out in an aqueous medium in the presence of usual nutrient substances. A suitable culture medium, for example, can contain a carbon source, an organic or inorganic nitrogen source, inorganic salts and growth factors. For the culture medium, glucose can be a preferred carbon source. Yeast extract can be a useful source of nitrogen. Phosphates, growth factors and trace elements can be added.

An illustrative example of a production process is provided in the Examples.

One skilled in the art will recognize that the ambroxide composition produced by such methods can be further purified and mixed with fragrant consumer products as described above.

The disclosure will be more fully understood upon consideration of the following non-limiting Examples. It should be understood that these examples, while indicating preferred embodiments of the subject technology, are given by way of illustration only. From the above discussion and these examples, one skilled in the art can ascertain the essential characteristics of the subject technology, and without departing from the spirit and scope thereof, can make various changes and modifications of the subject technology to adapt it to various uses and conditions.

### EXAMPLES

### Example 1: Ambroxide Synthesis Using Modified SHCs

Ambroxide is one of the key components of ambergris which is a solid, waxy, flammable substance of a dull grey or blackish color produced in the digestive system of sperm whales. It is thought to be produced as a secretion response to constant irritation caused by sharp beaks of squids and cuttlefish and their indigestible parts. Fresh ambergris is black, semi-viscous and almost smells of fecal matter and is of no value in perfumery, but as it is aged through years of exposure to sunlight, air, and the ocean, it oxidizes and hardens to a pleasantly aromatic substance which is found floating on the surface of the sea. For obvious reasons, this process of producing it is not a sustainable or practical one whatsoever. Currently, ambroxide is produced by multiple companies via a semi-synthetic pathway in which sclareol is produced biosynthetically then oxidatively degraded to a lactone and then hydrogenated to the corresponding diol. The resulting compound is dehydrated to form ambroxide. It has been shown, however, that an alternative biosynthetic route to ambroxide production is available via substrate promiscuity of squalene-hopene cyclases (SHCs).

### Materials and Methods

### Bacterial Strains and Plasmids

E. coli strains of 10G and C41 (DE3) were purchased from Lucigen. 10G cells were used for the cloning and propagation of plasmids, while C41 cells were used for the expression of enzyme and bioconversion thereafter. Plasmid pET28-ntHisYTK01 (FIG. 1) was constructed with the nucleotide sequence listed as SEQ ID NO: 1. This plasmid was used for both gene cloning and gene expression purposes.

### DNA Manipulation

### Cloning

Golden gate cloning was employed to insert genes of interest into the pET28-ntHisYTK01 expression vector using standard procedure outlined by New England Biolabs (NEB). The Q5 PCR system by NEB was used for the amplification of any genes of interest prior to cloning. All reagents for golden gate cloning were purchased from NEB.

### Mutagenesis

REPLACR-mutagenesis was performed to introduce site directed point mutations at residues of interest. Primers were ordered from Genewiz and the Q5 PCR system from NEB was used for the amplification of genes.

### Circular Permutation

Circular permutants were constructed by first amplifying specific segments of the gene of interest via Q5 PCR, then reassembling those segments to produce the desired rearranged variant using circular polymerase extension cloning. Assembled constructs were then transformed into 10G cells for propagation and the resulting plasmids after miniprep were sequence verified via sanger sequencing offered by Genewiz.

### Identification of Target Genes

Protein sequences from all of the proteins that have been previously shown to produce ambroxide from homofarnesol including BjaSHC1 (A5EBP6), AacSHC1 (P33247), ZmoSHC1 (Q5NM88), ZmoSHC2 (P33990), RpaSHC1 (Q07I43), and GmoSHC1 (G6XHN3) were used to propagate sequence similarity networks using the online EFI tool efi.igb.illinois.edu/efi-est/. The sequence similarity networks that were propagated were then visualized and merged into one larger network using Cytoscape. The E-value for this merged network was then increased until the characterized enzymes with desired function began to show more stringent clustering with other uncharacterized enzymes. This occurred at an E-value of 225. Uncharacterized enzymes that clustered closely with those that have been functionally characterized were selected for functional characterization. The selected enzymes were codon optimized for E. coli and synthesized by TWIST Bioscience.

### Construction of Plasmids

E. coli codon optimized gene sequences derived from the protein sequences within the sequence similarity network were synthesized by TWIST biosciences. These genes were then cloned into pET28-ntHisYTK01 using BsaI golden gate cloning as described in the manufactures protocol (NEB). Cloned constructs were then transformed into 10G cells for propagation and plated for colony isolation. Single colonies were then selected for miniprep and were grown up in 5 mL LB cultures at 37° C. The following day plasmid isolation was performed using the IBI Scientific Hi-Speed Mini Plasmid Kit, and the plasmids were sequenced by Genewiz for sequence verification.

Expression of Homofarnesol-Ambroxide Cyclase in E. coli C41 Cells

Sequence verified pET28-ntHisYTK01 constructs were transformed into C41 cells using standard protocol and let incubate at 37° C. overnight following plating. For each transformant, 4 colonies were selected for inoculation into 5 mL LB precultures and were let incubate overnight at 37° C. and 250 RPM. The following day, 50 mL M9CL media (lx M9 Salts [12.8 g/L Na₂HPO₄*7H₂O, 3 g/L KH₂PO₄, 0.5 g/L NaCl, lg/L NH₄Cl], 12 g/L Tryptone, 8 g/L Yeast Extract, 10 g/L Glycerol, 10 mg/L Thiamine, 2.5 mM MgSO₄, 1 mM CaCl₂, 1× Trace Metals [4.84 mg/L Na₂EDTA*2H₂O, 2.3 mg/L ZnSO₄*7H₂O, 1.11 mg/L H₃BO₃, 0.51 mg/L MnCl₂*4H₂O, 0.17 mg/L CoCl₂*6H₂O, 0.15 mg/L CuSO₄*5H₂O, 0.10 mg/L (NH₄)₆Mo₇O₂₄*4H₂O], pH 7.0) cultures were inoculated with 120 μL from their respective preculture and were then let shake at 37° C. until an O.D. 600 value of 0.6 was reached. Expression was then induced via addition of 1 mM IPTG and the cultures were let shake overnight at 37° C. and 250 RPM.

### Bioconversion of Homofarnesol to Ambroxide in Shake Flasks

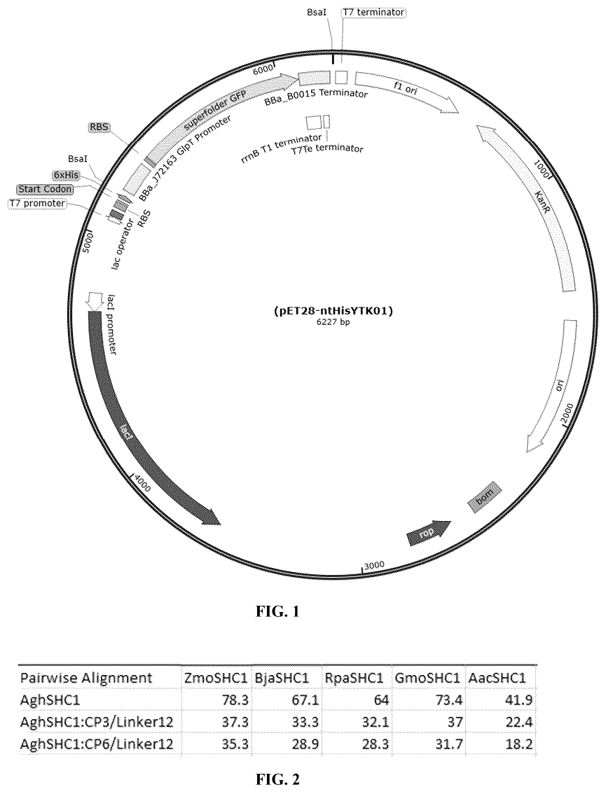

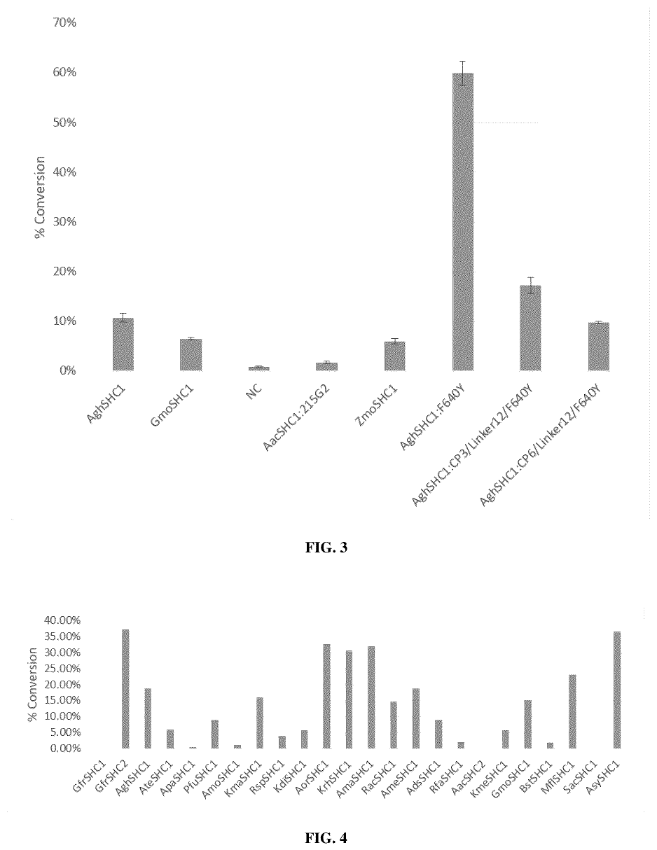

Following expression of homofarnesol-ambroxide cyclases in C41 cells, these expression cultures were spun down at 4000×g for 20 minutes and the supernatant was discarded. The cell pellets were the resuspended to 250 g/L in Bioconversion Media (1× M9 Salts [12.8 g/L Na₂HPO₄*7H₂O, 3 g/L KH₂PO₄, 0.5 g/L NaCl, 1 g/L NH₄Cl], 5 g/L Yeast Extract, 5 g/L glucose, 100 mM citric acid buffer [5.104 g/L anhydrous citric acid, 21.59 g sodium citrate dihydrate], pH 5.4). Cell resuspensions were then transferred to glass tubes and homofarnesol was added to 10 g/L and SDS was added to a final concentration of 0.1%. These resuspensions were let shake at 37° C. and 250 RPM overnight to allow for enzymatic whole cell bioconversion of homofarnesol to ambroxide. Comparison of enzymes under these conditions can be found in FIG. 2.

### Extraction of Metabolites

Following whole cell bioconversion, the metabolites were extracted for analysis using hexanes. 200 μL from each whole cell bioconversion was aliquoted into its respective microcentrifuge tube where it was extracted using 800 μL of hexanes. The mixture was vortexed for 15 seconds and then briefly centrifuged to aid in phase separation. The hexane layer was then extracted into GC vials for analysis.

### GC-MS/FID Analysis of Metabolite Extracts

Extracts were quantified on a SHIMADZU GC-2014 GC-FID with a Zebron ZB-1HT column (30 m Length, 0.25 mm Internal Diameter, 0.10 μm Film Thickness). Splitless injections of 1 μL of sample were used with a medium temperature Shimazu septa (221-76650-01) and a splitless Shimadzu inlet liner (221-48876-02) at an inlet temperature of 225° C. N2 gas was used at a column flow of 1.45 mL/min. Upon injection, an initial column temperature of 90° C. was held for 1 minute thereafter increasing the temperature to 160° C. at a rate of 20° C./min followed by an increase to 280° C. at a rate of 30° C./min. This temperature was held for a minute before equilibrating for the next injection. The FID detector was set to a temperature of 300° C. and a sample rate of 40 msec. A standard curve was made for both ambroxide as well as homofarnesol which allowed for the determination of ambroxide and homofarnesol concentrations in the sample.

Extracts were assessed for purity using a Nexis GC-2030 with a MS-QP2020NX. A 30 m Rtx-5MS column with an inner diameter of 0.25 mm was used. The analysis method uses a 1:10 split injection at 265° C. with a column flow of 1.35 mL/min. Column injection temperature is set to 80° C. with a hold time of 3 min. Following this period of time, the column temperature is ramped up to 260° C. at a rate of 45° C. per minute. The MS is set to only scan for ions within the range of 50-400 m/z with a 2000 scan speed. Analysis via MS only begins after 6 minutes of the run. Retention time and mass spectra of the extracts run using this method were compared to runs using authentic standards for both ambroxide as well as homofarnesol.

### Results

### Enzymatic Conversion of Homofarnesol to Ambroxide

Upon analysis of extracts obtained using the described methods and comparisons of them with authentic standards, it was clear that enzymes selected for characterization including AghSHC1, AteSHC1, ApaSHC1, PfuSHC1, KdiSHC1, AorSHC1, KrhSHC1, AmaSHC1, RacSHC1, AmeSHC1, AdsSHC1, RfaSHC1, KmeSHC1, MflSHC1, AsySHC1, BstSHC1, and GfrSHC2 were all capable of producing ambroxide from homofarnesol to various degrees.

### Comparison of Top Conagen Strains to the Competition

This experiment was performed by first growing up 75 mL cultures in triplicate for each strain to an O.D. 600 value of 0.6 at 37° C. while shaking at 250 RPM before inducing with 1 mM IPTG and allowing the cultures to grow and express protein overnight. The following morning the cultures were pelleted and the cell pellets were resuspended to cell densities of 250 g/L. 2 mL from each suspension was then aliquoted into a 24 well plate where SDS was added to a final concentration of 0.1% and homofarnesol substrate was added to a final concentration of 10 g/L. The plate was then let shake at 37° C. and 250 RPM for 24 hours. Conversion was then measured by taking 200 μL aliquots from each bioconversion and extracting using 800 μL of hexanes prior to analyzing on a GC-FID with which standard curves for both homofarnesol and ambroxide were made prior. Concentration of present homofarnesol and ambroxide in the extracts were then calculated using the standard curve and percent conversion was determined as a function of total ambroxide divided by total metabolite detected.

### REFERENCES

Neumann, S., & Simon, H. (1986). Purification, Partial Characterization and Substrate Specificity of a Squalene Cyclase from Bacillus acidocaldarius. Biological Chemistry, 367(2), 723-730. doi.org/10.1515/bchm3.1986.367.2.723
Seitz, M., Klebensberger, J., Siebenhaller, S., Breuer, M., Siedenburg, G., Jendrossek, D., & Hauer, B. (2012). Substrate specificity of a novel squalene-hopene cyclase from Zymomonas mobilis. Journal of Molecular Catalysis B: Enzymatic, 84, 72-77. doi.org/10.1016/j.molcatb.2012.02.007
Eichhorn, E., Locher, E., Guillemer, S., Wahler, D., Fourage, L., & Schilling, B. (2018). Biocatalytic Process for (−)-Ambrox Production Using Squalene Hopene Cyclase. Advanced Synthesis & Catalysis, 360(12), 2339-2351. doi.org/10.1002/adsc.201800132

### Example 2: Circular Permutants

Circular permutation is a technique that has been shown to be a feasible strategy for altering the primary sequence without significantly effecting tertiary structure. Luckily, the N- and C-termini of SHCs are adjacent to one another being only 13.2 angstroms apart in the tertiary structure allowing this technique to be applicable. Therefore, in theory, fusing these nearby termini and cutting the primary sequence elsewhere at various external loops should not significantly alter the tertiary structure and therefore the catalytic activity of the enzyme. Due to the fact that the reaction this enzyme is being used to catalyze is not the reaction it has evolved to perform in nature, it may be possible that this type of engineering will even increase the catalytic activity when using homofarnesol as a substrate.

TABLE X2
Linker sequences selected for the fusion of the
native N- and C-terminus

| Linker ID | Amino Acid Sequence | SEQ ID NO: |
|---|---|---|
| CN_Linker_1 | GGGGGG | 40 |
| CN_Linker_2 | GGGGGGGGGG | 41 |
| CN_Linker_3 | GGGGGGGGGGGG | 42 |
| CN_Linker_4 | GGSGGTGGSGG | 43 |
| CN_Linker_5 | GGSGREEKGSGG | 44 |
| CN_Linker_6 | GGGSTSGGG | 45 |

TABLE X3
Linker sequences selected for the fusion of the
native N- and C-terminus round 2

| Linker ID | Amino Acid Sequence | SEQ ID NO: |
|---|---|---|
| CN_Linker_7 | AVNRPQDHEA | 46 |
| CN_Linker_8 | AIGEDMTQP | 47 |
| CN_Linker_9 | LGLPRETP | 48 |
| CN_Linker_10 | AGAEERYTP | 49 |
| CN_Linker_11 | ACRHREEWK | 50 |
| CN_Linker_12 | GGGSGGGS | 51 |
| CN_Linker_13 | GGGSGGGSGGGS | 52 |
| CN_Linker_14 | GGGSGGGSGGGP | 53 |

### Materials and Methods

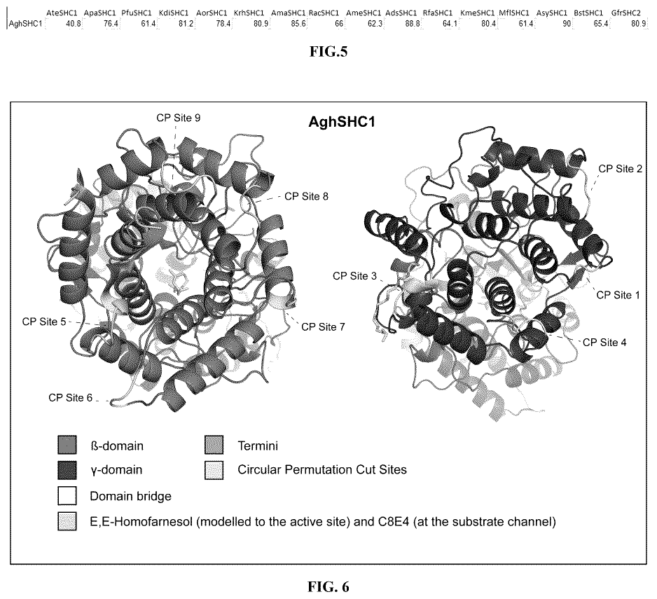

In order to visualize the structure of SEQ ID NO: 3 from a publicly available SHC structure (pdb 2SQC), SEQ ID NO: 3 was modelled using SWISS-MODEL. This model was then used to identify loop regions that are away from conserved domains and motifs and are located near the outside of the enzyme since cutting at these regions should have the lowest negative impact on protein folding. Nine candidate cut sites were selected for the introduction of new termini (FIG. 6).

After selecting the loop regions where cuts were to be made, the specific amino acid residues in those loops where the cuts were to be introduced were selected. Generally, the residues that were selected to make cuts were those that were perceived to have the least involvement in enzyme secondary structure, have the lowest conservation patterns, and are amino acids that exhibit relatively higher freedom in phi psi angles. Once specific terminal residues were identified at each location, the two residues “GA” were planned to be appended to the new C-terminus, while the two residues “MA” were planned to be appended to the new N-terminus. Primers were design for each cut site to include this design and to provide homology to the vector backbone that would be used, pET28-ntHisYTK01.

In order to fuse the native N- and C-termini, truncations of the existing termini were designed so that all excess amino acids past predicted secondary structure would be deleted. This was done so that various iteration of linker sequences between the termini could be screened. Various linker sequences for fusing the termini were then designed with a focus towards flexibility, similarities to naturally observed loop sequences, and lengths that are appropriate for the distance between the two termini (Table X2). These linkers were ordered as oligomers with overhangs homologous to the N- and C terminus- of SEQ ID NO: 3 for cloning. Lastly, primers were designed for the fusion of the termini to the linkers, and primers were designed for amplification of the backbone so that all of the fragments for plasmid assembly could be generated.

### Results

Out of the 54 circular permutants that were screened for activity, only those that were cut at CP Site 3 and CP Cut Site 6 retained the desired activity regardless of the linker that was utilized to fuse the native N- and C-termini. In an attempt to increase activity, 8 more linker sequences (Table X3) were screened for both CP Cut Site 3 and CP Cut Site 6, and the 16 additional circular permutants were screened. Out of all of the circular permutants, CP3/Linker12 (SEQ ID NO: 37) and CP6/Linker12 (SEQ ID NO: 39) were shown to be the most active.

Nucleic Acid and Amino Acid Sequences

SEQ ID NO: 1 pET28-ntHisYTK01 NT Plasmid
GCTGAGCAATAACTAGCATAACCCCTTGGGGCCTCTAAACGGGTCTTGAGGGGTTTT
TTGCTGAAAGGAGGAACTATATCCGGATTGGCGAATGGGACGCGCCCTGTAGCGGC
GCATTAAGCGCGGCGGGTGTGGTGGTTACGCGCAGCGTGACCGCTACACTTGCCAG
CGCCCTAGCGCCCGCTCCTTTCGCTTTCTTCCCTTCCTTTCTCGCCACGTTCGCCGGCT
TTCCCCGTCAAGCTCTAAATCGGGGGCTCCCTTTAGGGTTCCGATTTAGTGCTTTACG
GCACCTCGACCCCAAAAAACTTGATTAGGGTGATGGTTCACGTAGTGGGCCATCGCC
CTGATAGACGGTTTTTCGCCCTTTGACGTTGGAGTCCACGTTCTTTAATAGTGGACTC
TTGTTCCAAACTGGAACAACACTCAACCCTATCTCGGTCTATTCTTTTGATTTATAAG
GGATTTTGCCGATTTCGGCCTATTGGTTAAAAAATGAGCTGATTTAACAAAAATTTA
ACGCGAATTTTAACAAAATATTAACGTTTACAATTTCAGGTGGCACTTTTCGGGGAA
ATGTGCGCGGAACCCCTATTTGTTTATTTTTCTAAATACATTCAAATATGTATCCGCT
CATGAATTAATTCTTAGAAAAACTCATCGAGCATCAAATGAAACTGCAATTTATTCA
TATCAGGATTATCAATACCATATTTTTGAAAAAGCCGTTTCTGTAATGAAGGAGAAA
ACTCACCGAGGCAGTTCCATAGGATGGCAAGATCCTGGTATCGGTCTGCGATTCCGA
CTCGTCCAACATCAATACAACCTATTAATTTCCCCTCGTCAAAAATAAGGTTATCAA
GTGAGAAATCACCATGAGTGACGACTGAATCCGGTGAGAATGGCAAAAGTTTATGC
ATTTCTTTCCAGACTTGTTCAACAGGCCAGCCATTACGCTCGTCATCAAAATCACTCG
CATCAACCAAACCGTTATTCATTCGTGATTGCGCCTGAGCGAGACGAAATACGCGAT
CGCTGTTAAAAGGACAATTACAAACAGGAATCGAATGCAACCGGCGCAGGAACACT
GCCAGCGCATCAACAATATTTTCACCTGAATCAGGATATTCTTCTAATACCTGGAAT
GCTGTTTTCCCGGGGATCGCAGTGGTGAGTAACCATGCATCATCAGGAGTACGGATA
AAATGCTTGATGGTCGGAAGAGGCATAAATTCCGTCAGCCAGTTTAGTCTGACCATC
TCATCTGTAACATCATTGGCAACGCTACCTTTGCCATGTTTCAGAAACAACTCTGGC
GCATCGGGCTTCCCATACAATCGATAGATTGTCGCACCTGATTGCCCGACATTATCG
CGAGCCCATTTATACCCATATAAATCAGCATCCATGTTGGAATTTAATCGCGGCCTA
GAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACT
GAGCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTTGC
CGGATCAAGAGCTACCAACTCTTTTTCCGAAGGTAACTGGCTTCAGCAGAGCGCAGA
TACCAAATACTGTCCTTCTAGTGTAGCCGTAGTTAGGCCACCACTTCAAGAACTCTG
TAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCGAACGAC
CTACACCGAACTGAGATACCTACAGCGTGAGCTATGAGAAAGCGCCACGCTTCCCG
AAGGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTTCG
CCACCTCTGACTTGAGCGTCGATTTTTGTGATGCTCGTCAGGGGGGCGGAGCCTATG
GAAAAACGCCAGCAACGCGGCCTTTTTACGGTTCCTGGCCTTTTGCTGGCCTTTTGCT
CACATGTTCTTTCCTGCGTTATCCCCTGATTCTGTGGATAACCGTATTACCGCCTTTG
AGTGAGCTGATACCGCTCGCCGCAGCCGAACGACCGAGCGCAGCGAGTCAGTGAGC
GAGGAAGCGGAAGAGCGCCTGATGCGGTATTTTCTCCTTACGCATCTGTGCGGTATT
TCACACCGCATATATGGTGCACTCTCAGTACAATCTGCTCTGATGCCGCATAGTTAA
GCCAGTATACACTCCGCTATCGCTACGTGACTGGGTCATGGCTGCGCCCCGACACCC
GCCAACACCCGCTGACGCGCCCTGACGGGCTTGTCTGCTCCCGGCATCCGCTTACAG
ACAAGCTGTGACCGTCTCCGGGAGCTGCATGTGTCAGAGGTTTTCACCGTCATCACC
GAAACGCGCGAGGCAGCTGCGGTAAAGCTCATCAGCGTGGTCGTGAAGCGATTCAC
AGATGTCTGCCTGTTCATCCGCGTCCAGCTCGTTGAGTTTCTCCAGAAGCGTTAATGT
CTGGCTTCTGATAAAGCGGGCCATGTTAAGGGCGGTTTTTTCCTGTTTGGTCACTGAT
GCCTCCGTGTAAGGGGGATTTCTGTTCATGGGGGTAATGATACCGATGAAACGAGA
GAGGATGCTCACGATACGGGTTACTGATGATGAACATGCCCGGTTACTGGAACGTTG
TGAGGGTAAACAACTGGCGGTATGGATGCGGCGGGACCAGAGAAAAATCACTCAGG
GTCAATGCCAGCGCTTCGTTAATACAGATGTAGGTGTTCCACAGGGTAGCCAGCAGC
ATCCTGCGATGCAGATCCGGAACATAATGGTGCAGGGCGCTGACTTCCGCGTTTCCA
GACTTTACGAAACACGGAAACCGAAGACCATTCATGTTGTTGCTCAGGTCGCAGAC
GTTTTGCAGCAGCAGTCGCTTCACGTTCGCTCGCGTATCGGTGATTCATTCTGCTAAC
CAGTAAGGCAACCCCGCCAGCCTAGCCGGGTCCTCAACGACAGGAGCACGATCATG
CGCACCCGTGGGGCCGCCATGCCGGCGATAATGGCCTGCTTCTCGCCGAAACGTTTG
GTGGCGGGACCAGTGACGAAGGCTTGAGCGAGGGCGTGCAAGATTCCGAATACCGC
AAGCGACAGGCCGATCATCGTCGCGCTCCAGCGAAAGCGGTCCTCGCCGAAAATGA
CCCAGAGCGCTGCCGGCACCTGTCCTACGAGTTGCATGATAAAGAAGACAGTCATA
AGTGCGGCGACGATAGTCATGCCCCGCGCCCACCGGAAGGAGCTGACTGGGTTGAA
GGCTCTCAAGGGCATCGGTCGAGATCCCGGTGCCTAATGAGTGAGCTAACTTACATT
AATTGCGTTGCGCTCACTGCCCGCTTTCCAGTCGGGAAACCTGTCGTGCCAGCTGCA
TTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCT
GAGAGAGTTGCAGCAAGCGGTCCACGCTGGTTTGCCCCAGCAGGCGAAAATCCTGT
TTGATGGTGGTTAACGGCGGGATATAACATGAGCTGTCTTCGGTATCGTCGTATCCC
ACTACCGAGATATCCGCACCAACGCGCAGCCCGGACTCGGTAATGGCGCGCATTGC
GCCCAGCGCCATCTGATCGTTGGCAACCAGCATCGCAGTGGGAACGATGCCCTCATT
CAGCATTTGCATGGTTTGTTGAAAACCGGACATGGCACTCCAGTCGCCTTCCCGTTC
CGCTATCGGCTGAATTTGATTGCGAGTGAGATATTTATGCCAGCCAGCCAGACGCAG
ACGCGCCGAGACAGAACTTAATGGGCCCGCTAACAGCGCGATTTGCTGGTGACCCA
ATGCGACCAGATGCTCCACGCCCAGTCGCGTACCGTCTTCATGGGAGAAAATAATA
CTGTTGATGGGTGTCTGGTCAGAGACATCAAGAAATAACGCCGGAACATTAGTGCA
GGCAGCTTCCACAGCAATGGCATCCTGGTCATCCAGCGGATAGTTAATGATCAGCCC
ACTGACGCGTTGCGCGAGAAGATTGTGCACCGCCGCTTTACAGGCTTCGACGCCGCT
TCGTTCTACCATCGACACCACCACGCTGGCACCCAGTTGATCGGCGCGAGATTTAAT
CGCCGCGACAATTTGCGACGGCGCGTGCAGGGCCAGACTGGAGGTGGCAACGCCAA
TCAGCAACGACTGTTTGCCCGCCAGTTGTTGTGCCACGCGGTTGGGAATGTAATTCA
GCTCCGCCATCGCCGCTTCCACTTTTTCCCGCGTTTTCGCAGAAACGTGGCTGGCCTG
GTTCACCACGCGGGAAACGGTCTGATAAGAGACACCGGCATACTCTGCGACATCGT
ATAACGTTACTGGTTTCACATTCACCACCCTGAATTGACTCTCTTCCGGGCGCTATCA
TGCCATACCGCGAAAGGTTTTGCGCCATTCGATGGTGTCCGGGATCTCGACGCTCTC
CCTTATGCGACTCCTGCATTAGGAAGCAGCCCAGTAGTAGGTTGAGGCCGTTGAGCA
CCGCCGCCGCAAGGAATGGTGCATGCAAGGAGATGGCGCCCAACAGTCCCCCGGCC
ACGGGGCCTGCCACCATACCCACGCCGAAACAAGCGCTCATGAGCCCGAAGTGGCG
AGCCCGATCTTCCCCATCGGTGATGTCGGCGATATAGGCGCCAGCAACCGCACCTGT
GGCGCCGGTGATGCCGGCCACGATGCGTCCGGCGTAGAGGATCGAGATCTCGATCC
CGCGAAATTAATACGACTCACTATAGGGGAATTGTGAGCGGATAACAATTCCCCTCT
AGAAATAATTTTGTTTAACTTTAAGAAGGAGATATACCATGGGCAGCAGCCATCATC
ATCATCATCACAGCAGCGGTATGGGAGACCGAAAGTGAAACGTGATTTCATGCGTC
ATTTTGAACATTTTGTAAATCTTATTTAATAATGTGTGCGGCAATTCACATTTAATTT
ATGAATGTTTTCTTAACATCGCGGCAACTCAAGAAACGGCAGGTTCGGATCTTAGCT
ACTAGAGAAAGAGGAGAAATACTAGATGCGTAAAGGCGAAGAGCTGTTCACTGGTG
TCGTCCCTATTCTGGTGGAACTGGATGGTGATGTCAACGGTCATAAGTTTTCCGTGC
GTGGCGAGGGTGAAGGTGACGCAACTAATGGTAAACTGACGCTGAAGTTCATCTGT
ACTACTGGTAAACTGCCGGTTCCTTGGCCGACTCTGGTAACGACGCTGACTTATGGT
GTTCAGTGCTTTGCTCGTTATCCGGACCATATGAAGCAGCATGACTTCTTCAAGTCC
GCCATGCCGGAAGGCTATGTGCAGGAACGCACGATTTCCTTTAAGGATGACGGCAC
GTACAAAACGCGTGCGGAAGTGAAATTTGAAGGCGATACCCTGGTAAACCGCATTG
AGCTGAAAGGCATTGACTTTAAAGAGGACGGCAATATCCTGGGCCATAAGCTGGAA
TACAATTTTAACAGCCACAATGTTTACATCACCGCCGATAAACAAAAAAATGGCATT
AAAGCGAATTTTAAAATTCGCCACAACGTGGAGGATGGCAGCGTGCAGCTGGCTGA
TCACTACCAGCAAAACACTCCAATCGGTGATGGTCCTGTTCTGCTGCCAGACAATCA
CTATCTGAGCACGCAAAGCGTTCTGTCTAAAGATCCGAACGAGAAACGCGATCATA
TGGTTCTGCTGGAGTTCGTAACCGCAGCGGGCATCACGCATGGTATGGATGAACTGT
ACAAATGACCAGGCATCAAATAAAACGAAAGGCTCAGTCGAAAGACTGGGCCTTTC
GTTTTATCTGTTGTTTGTCGGTGAACGCTCTCTACTAGAGTCACACTGGCTCACCTTC
GGGTGGGCCTTTCTGCGTTTATAGGTCTCTAGCG
SEQ ID NO: 2 AghSHC1 NT Organism: Acetobacter ghanensis
ATGAATACGATCTCTCATCCGAGTAAAGTGAAAGCAGCCGTTAGTGATAGCCACACT
CCCACCTCAGCGACGCCGACCCCATTCAAGGGTATGGACAACTCGCTTGCGCACAC
GGTCGCAGCGGCGTGTGATTGGCTGATCGGGGAGCAGAAAGCCGATGGACATTGGG
TGGGTCCGGTAGCGTCCAATGCATCAATGGAAGCGGAATGGTGCCTGGCTTTATGGT
ACCTGGGTCTGGAAGATCACCCGCTGCGCCCACGTTTAGGCAACGCCCTGTTGCAGA
TGCAGCGAGAGGACGGTTCTTGGGGCATTTACTGGGGCGCCGGGAACGGTGACATT
AATGCAACGGTAGAAGCTTACGCGGCACTACGTTCGTTAGGCTACCCGGCTGACAC
GCCAGCTTTAAGCAAAGCGTGTGCGTGGATTATGCGTATGGGCGGCCTGCGCAATAT
CCGCGTGTTCACTCGTTACTGGCTCGCGCTGATTGGCGAATGGCCGTGGGAACAGAC
GCCGAACCTGCCGCCGGAGATCATCTGGTTTCCGAATAAATTCGTTTTCAGCATTTA
CAACTTTGCACAGTGGGCCCGTGCAACATTAGTGCCTCTGGCAATTCTGAGTGCAAG
ACGCCCGTCACGTCCTCTCCGCCCGCAGGATCGTCTGGATGCGTTGTTCCCGGGCGG
TCGGGAAAATTTCGACTATGTGCTGCCTAAACGCGATGGCATGGACCTGTGGTCCTC
ATTCTTTCGCACCACGGACCGCGGCCTGCACTGGCTGCAATCAAAATTTCTGAAACG
CAACACCCTTCGCGAAGCCGCGATTAAACACATGCTGGAATGGATTATTCGCCATCA
GGACGCAGACGGCGGTTGGGGCGGCATTCAGCCGCCATGGGTGTACGGCTTAATGG
CACTACATGGTGAAGATTATCAGTTCCACCATCCGGTGATGGCTAAGGCTTTAGCCG
CCTTAGACGATCCTGGCTGGCGTCAAGATCGTGGGGATGCCAGCTGGGTGCAGGCC
ACGAACAGTCCAGTATGGGATACGATGTTAGCACTGATGGCGCTCCACGACGCGGG
CGCTGAAGAGCGCTATACCCCTGAAATGGATAAAGCGCTGGATTGGCTGTTACAGC
GCCAGGTTCGGGTAACAGGTGACTGGTCAATCAAACTGCCGGGAGTCGAACCTGGC
GGTTGGGCATTCGAATATGCGAATGACCGTTATCCCGATACCGACGATACCGCGGTT
GCGCTCATTGCACTGAGCGCGTGTCGCCACCGGGAAGAATGGAAGAAGAAAGGGGT
CGAAGCTGCGATCACACGCGGAGTAAATTGGCTCATTGCCATGCAGTCAACGTGCG
GCGGCTGGGGCGCATTTGATAAAGATAACAATCGTTCGTTACTGTCGAAGATTCCAT
TTTGTGACTTTGGCGAAGCTCTGGACCCACCCAGCGTCGATGTCACGGCACATGTGC
TTGAAGCCTTTGGTCTGCTGGGTCTGCCCCGCGAAACCCCTTCCATTCAGCGCGGCC
TTGCTTACATCAGAGCGGAACAGGAGCCGTCTGGAGCTTGGTTTGGCCGATGGGGC
GTTAATTACCTGTATGGGACCGGGGCAGTCCTACCGGCCCTGGCGGCAATCGGTGAG
GATATGACCCAGCCGTACATCACTCGTGCGTGTGATTGGCTCGTCGCTCATCAGCAG
GAAAACGGTGGCTGGGGTGAATCGTGTGCGTCATATATGGACGTCGCGTCGATTGGT
CATGGAACTCCGACCGCCTCTCAGACCGCATGGGCCCTGATGGGACTGATTGCGGTG
AATCGACCGCAGGATCACGAGGCAATCGCACGCGGCTGTCGTTTTCTGATTGACCGT
CAGGAAGAAGATGGAAGTTGGACCGAAGAGGAATTTACCGGTACCGGCTTTCCAGG
CTACGGTGTCGGCCAGACTATCAAACTGGACGATCCAGCCGTCGCTAAACGTCTGCA
ACAGGGTGCGGAACTTTCGCGTGCGTTCATGCTACGCTACGACCTGTATCGTCAGTT
CTTCCCGCTGATGGCCCTGAGCCGTGCGGCGCGTATCATTCCAATTAAGAAC
SEQ ID NO: 3 AghSHC1 AA Organism: Acetobacter ghanensis
MNTISHPSKVKAAVSDSHTPTSATPTPFKGMDNSLAHTVAAACDWLIGEQKADGHWV
GPVASNASMEAEWCLALWYLGLEDHPLRPRLGNALLQMQREDGSWGIYWGAGNGDI
NATVEAYAALRSLGYPADTPALSKACAWIMRMGGLRNIRVFTRYWLALIGEWPWEQTP
NLPPEIIWFPNKFVFSIYNFAQWARATLVPLAILSARRPSRPLRPQDRLDALFPGGRENFD
YVLPKRDGMDLWSSFFRTTDRGLHWLQSKFLKRNTLREAAIKHMLEWIIRHQDADGG
WGGIQPPWVYGLMALHGEDYQFHHPVMAKALAALDDPGWRQDRGDASWVQATNSP
VWDTMLALMALHDAGAEERYTPEMDKALDWLLQRQVRVTGDWSIKLPGVEPGGWAF
EYANDRYPDTDDTAVALIALSACRHREEWKKKGVEAAITRGVNWLIAMQSTCGGWGA
FDKDNNRSLLSKIPFCDFGEALDPPSVDVTAHVLEAFGLLGLPRETPSIQRGLAYIRAEQE
PSGAWFGRWGVNYLYGTGAVLPALAAIGEDMTQPYITRACDWLVAHQQENGGWGES
CASYMDVASIGHGTPTASQTAWALMGLIAVNRPQDHEAIARGCRFLIDRQEEDGSWTEE
EFTGTGFPGYGVGQTIKLDDPAVAKRLQQGAELSRAFMLRYDLYRQFFPLMALSRAARI
IPIKN
SEQ ID NO: 4 AteSHC1 NT Organism: Alicyclobacillus tengchongensis
ATGACCAAACAGCTGGCTGAAATCCCGGCGTATATGCAAACCCTGGATAACGGGGT
CGAGTACCTGTTGAGTCGTCAACATGAGGAAGGCTATTGGTGGGGTCCGCTTCTCTC
GAATGTAACTATGGAAGCGGAATATGTTCTGCTGTGCCATTGCTTGGGAAAAGTTGA
TAAAGAACGCCTGGAGAAAATCAAAACGTACTTACTGCACGAACAACGCGAAGATG
GAACTTGGGCTCAGTACCCAGGTGGTCCGCAGGACCTGGACACGACTATCGAAGCG
TACGTGGCACTGAAATATATTGGTTTATCGCCTGACGACGAACGTATGCAGAAAGCG
CTGGCTTTCATCCAGAGTCAGGGTGGCATTGAATCGGCGCGGGTGTTTACCCGGCTC
TGGCTGGCACTGGTAGGGGAATACCCATGGCGTAAACTGCCGGTTGTGCCACCCGA
AATCATGTTTCTCGGGAAGAACATGCCTTTAAATATTTATGACTTCGGCAGCTGGGC
TCGTCCAACTATCGTTGCCCTTACTATCGTTATGAGTCGGCGGGCGGTGTTTCCCCTG
CCAGCCCATGCGAAAGTTCCCGAGTTATTCGAAACGAACGTTCCGCCACGCCGTCGT
GCCGCGAAAGGCGGCAACAGTAGTCTGTTTCTGTCCATCGACAAACTGCTGCAAGG
ATACCAGAACGGCTCCTTCCATCCATTCCGCAAAGCCGCTGAGCAACGCGCGATCG
AGTGGCTAATCGAACATCAAGCCGGTGATGGTAGCTGGGGCGGGATCCAGCCGCCG
TGGTTTTACGCACTGTTGGCTCTTAAAGTGATGAACATGACCTCGCATCCGGCCTTTA
TTAAAGGCTGGGAAGGTCTGGAGTTATATGGCCTGGAACTGGAATACGGTGGGTGG
ATGTTTCAGGCGTCTATCTCCCCGGTGTGGGATACCGGCCTGAGCATCCTTGCCCTG
CGCGCCGCGGGACTCGCCCCTGATGAACCGGCGCTTGTCAAAGCAGGTCAGTGGCT
GCTGGATCATCGCATCGCAACAAAAGGGGATTGGGCAGTGCGCCGTCCGAATGCCA
AACCTGGTGGTTGGGCCTTCCAGTTTGATAATCCCCATTATCCGGACGTCGATGATA
CGGCCGTGGTTGTCTGGGCCCTTAACGGCCTGAAACTGCCCAATGAGGCGGAACGT
CGTGACGCCATGACGGCCGGTTTTCGTTGGCTGACCGCCATGCAGTCGTCTAACGGT
GGCTGGGGCGCGTATGATGTGGATAACAACAAAGAGTTACCTAATCGCATCCCGTTT
TGCGATTTTGGCGAAGTCATTGATCCGCCTAGTGAAGATGTTACCGCGCACGTCCTT
GAATGTTTCGGTTCCTTTGGATATGACGAAGCCTGGAAAGTTGTTGCGCGTGCTGTC
AACTACCTGAAACGGGAACAGAAACCGGACGGCTCCTGGTACGGCCGTTGGGGCGT
TAATTACATTTATGGTATTGGCGCGGTTGTCCCGGCATTGAAATCGGTCGGAGTGGA
CATGAAAGAACCGTTCGTGCAGAAAGCGCTAGACTGGTTGGTTGCACATCAGAACG
AAGATGGTGGCTGGGGTGAAGATTGTCGCAGCTATGTCGATGAACGGTTTGCGGGC
GTAGGACCCAGCACACCCAGTCAAACGGCCTGGGCCCTGATGGCACTGATTGCCGG
CGGTCGTGTGCAAGCGGATGCAGTGTCACGCGGCGTTGCGTATTTGGTGCGCACGCA
GCGTCCGGATGGTGGCTGGGACGAACCATATTACACCGGCACTGGTTTTCCCGGCGA
CTTTTATTTAGGCTATACTCTTTATCGCCACATCTTTCCGGTCATGGCCCTGGGCCGC
TACAAAGATGCTCTGGGCCGGCTTACGCGT
SEQ ID NO: 5 AteSHC1 AA Organism: Alicyclobacillus tengchongensis
MTKQLAEIPAYMQTLDNGVEYLLSRQHEEGYWWGPLLSNVTMEAEYVLLCHCLGKVD
KERLEKIKTYLLHEQREDGTWAQYPGGPQDLDTTIEAYVALKYIGLSPDDERMQKALAF
IQSQGGIESARVFTRLWLALVGEYPWRKLPVVPPEIMFLGKNMPLNIYDFGSWARPTIVA
LTIVMSRRAVFPLPAHAKVPELFETNVPPRRRAAKGGNSSLFLSIDKLLQGYQNGSFHPF
RKAAEQRAIEWLIEHQAGDGSWGGIQPPWFYALLALKVMNMTSHPAFIKGWEGLELYG
LELEYGGWMFQASISPVWDTGLSILALRAAGLAPDEPALVKAGQWLLDHRIATKGDWA
VRRPNAKPGGWAFQFDNPHYPDVDDTAVVVWALNGLKLPNEAERRDAMTAGFRWLT
AMQSSNGGWGAYDVDNNKELPNRIPFCDFGEVIDPPSEDVTAHVLECFGSFGYDEAWK
VVARAVNYLKREQKPDGSWYGRWGVNYIYGIGAVVPALKSVGVDMKEPFVQKALDW
LVAHQNEDGGWGEDCRSYVDERFAGVGPSTPSQTAWALMALIAGGRVQADAVSRGV
AYLVRTQRPDGGWDEPYYTGTGFPGDFYLGYTLYRHIFPVMALGRYKDALGRLTR
SEQ ID NO: 6 ApaSHC1 NT Organism: Acetobacter pasteurianus
ATGAATATGGCGTCACGTTTCTCGCTCAAGAAGATCCTGCGGAGCGGCTCAGATACG
CAGGGTACCAATGTTAACACCCTGATACAAAGCGGCACATCTGATATTGTGCGCCAG
AAACCGGCTCCGCAGGTACCCGCGGACCTGTCCGCATTAAAAGCCATGGGCAACAG
CTTGACGCACACCCTGTCAAGCGCGTGTGAGTGGCTGATGAAACAGCAGAAACCGG
ATGGCCACTGGGTTGGCAGTGTTGGGTCTAATGCGAGCATGGAAGCAGAGTGGTGC
CTGGCCCTGTGGTTTCTGGGCCTTGAAGATCATCCGCTGCGTCCTCGCCTGGGTAAG
GCACTGTTAGAAATGCAGCGTCCGGACGGTTCGTGGGGAACCTATTATGGAGCGGG
ATCGGGCGATATTAACGCTACGGTAGAATCATACGCCGCACTGCGGAGTCTGGGCT
ATGCGGAAGATGATCCGGCCGTTTCGAAAGCCGCCGCTTGGATAATCTCGAAGGGC
GGATTGAAGAATGTGCGTGTGTTTACACGCTATTGGCTGGCACTTATCGGTGAATGG
CCGTGGGAGAAAACCCCGAATCTGCCACCGGAAATTATCTGGTTCCCTGACAATTTT
GTGTTTAGCATCTATAACTTTGCGCAGTGGGCGCGTGCGACGATGATGCCACTTGCC
ATTCTAAGTGCACGTCGTCCATCACGCCCACTTCGCCCACAGGACCGCCTGGATGCA
CTATTCCCTGGTGGTCGCGCCAATTTTGATTATGAACTGCCGACCAAAGAAGGCAGG
GACGTAATCGCAGATTTCTTCCGTCTTGCTGATAAAGGTCTGCACTGGCTGCAAAGT
TCATTTCTGAAACGTGCACCGTCACGTGAGGCAGCCATTAAGTATGTGCTAGAATGG
ATTATCTGGCACCAGGATGCAGACGGCGGTTGGGGCGGTATTCAGCCGCCGTGGGT
ATACGGTCTGATGGCGCTGCACGGCGAAGGCTACCAATTTCATCACCCGGTGATGGC
CAAAGCGCTGGACGCGCTAAATGACCCCGGGTGGCGTCATGATAAGGGTGACGCTA
GCTGGATCCAAGCGACGAACAGCCCTGTTTGGGATACTATGTTGAGCCTGATGGCGC
TGCACGACGCAAATGCGGAAGAACGCTTTACCCCGGAAATGGATAAAGCTCTGGAC
TGGCTGCTGTCACGCCAGATCCGCGTTAAGGGTGACTGGAGCGTCAAACTGCCTAAC
ACAGAACCGGGTGGTTGGGCATTCGAATACGCGAACGACCGCTATCCAGACACGGA
CGACACCGCTGTGGCCCTGATTGCGATTGCCTCATGTCGTAATAGGCCGGAATGGCA
AGCAAAAGGCGTCGAAGAAGCGATTGGCAGGGGTGTACGTTGGCTCGTTGCTATGC
AATCCTCGTGCGGCGGCTGGGGTGCGTTTGACAAAGATAACAACAAGTCAATACTG
GCGAAAATTCCGTTCTGTGATTTCGGTGAGGCCCTCGACCCACCGAGCGTGGACGTT
ACTGCACATGTCCTCGAGGCCTTCGGTTTGCTCGGGTTGCCAAGAGATTTACCGTGC
ATTCAGCGCGGGTTAGCGTACATTCGTAAAGAACAGGATCCTACGGGCCCGTGGTTT
GGCCGATGGGGAGTAAATTACCTGTATGGGACGGGCGCTGTACTTCCAGCGTTGGCC
GCCCTCGGTGAGGACATGACACAACCGTACATTTCCAAAGCGTGTGATTGGCTGATA
AACTGTCAGCAGGAAAACGGCGGGTGGGGAGAAAGTTGTGCGAGCTACATGGAAGT
TAGCTCCATCGGGCACGGCGCAACCACGCCTTCACAGACCGCCTGGGCGCTGATGG
GCCTCATTGCTGCAAATCGGCCGCAGGACTACGAAGCGATTGCCAAGGGTTGTCGAT
ACTTAATTGATCTTCAGGAAGAGGACGGCAGCTGGAACGAAGAAGAGTTCACCGGG
ACAGGCTTCCCAGGCTATGGCGTGGGGCAAACCATCAAACTCGATGATCCTGCTATT
TCTAAACGTCTGATGCAAGGCGCCGAATTGAGCCGCGCGTTCATGCTGCGTTATGAT
CTGTATCGCCAACTCTTCCCGATTATCGCCCTCTCACGCGCATCCCGTCTGATCAAAC
TGGGAAAT
SEQ ID NO: 7 ApaSHC1 AA Organism: Acetobacter pasteurianus
MNMASRFSLKKILRSGSDTQGTNVNTLIQSGTSDIVRQKPAPQVPADLSALKAMGNSLT
HTLSSACEWLMKQQKPDGHWVGSVGSNASMEAEWCLALWFLGLEDHPLRPRLGKALL
EMQRPDGSWGTYYGAGSGDINATVESYAALRSLGYAEDDPAVSKAAAWIISKGGLKNV
RVFTRYWLALIGEWPWEKTPNLPPEIIWFPDNFVFSIYNFAQWARATMMPLAILSARRPS
RPLRPQDRLDALFPGGRANFDYELPTKEGRDVIADFFRLADKGLHWLQSSFLKRAPSRE
AAIKYVLEWIIWHQDADGGWGGIQPPWVYGLMALHGEGYQFHHPVMAKALDALNDP
GWRHDKGDASWIQATNSPVWDTMLSLMALHDANAEERFTPEMDKALDWLLSRQIRV
KGDWSVKLPNTEPGGWAFEYANDRYPDTDDTAVALIAIASCRNRPEWQAKGVEEAIGR
GVRWLVAMQSSCGGWGAFDKDNNKSILAKIPFCDFGEALDPPSVDVTAHVLEAFGLLG
LPRDLPCIQRGLAYIRKEQDPTGPWFGRWGVNYLYGTGAVLPALAALGEDMTQPYISK
ACDWLINCQQENGGWGESCASYMEVSSIGHGATTPSQTAWALMGLIAANRPQDYEAIA
KGCRYLIDLQEEDGSWNEEEFTGTGFPGYGVGQTIKLDDPAISKRLMQGAELSRAFMLR
YDLYRQLFPIIALSRASRLIKLGN
SEQ ID NO: 8 PfuSHC1 NT Organism: Phaeospirillum fulvum
ATGACGAACCAACGCACGCGGCTCAAAGTGGCCTCGGAACGCGCTGATGAGATTGA
TACAGACGGTGATCGAACGGCAACTCTTCCGGTCAGCCCGGTTGTGAACAGCGGCA
AGAACTCGGCACCAATCGGTTTGGCCGCGAACACCGACTCTAGCCTCACGGTTAAA
ATTAAAGCAGCCATTAACGCGGCTGGAGATTGGTTGCTGGACCGCCAGAACGAGGA
CGGTCATTGGGTAGGTCCGTTGCAGAGCAATGCATGCATGGAAGCTCAGTGGTGTCT
GGCGTTATGGTTCCTGGGCTTAGAGAACCACCCTCTCCGCCCTCGCCTCGGACAGTC
ACTCCTGGAAACGCAGCGTGAAGATGGCTCATGGGAAGTCTACTATGCTGCCTCTGC
GGGCGACATCAACACAACCGTGGAAGCGTACGCCGCGCTGCGTTCCCTTGGCTTCCC
GGAAAGTGATCCGCGTCTGACGAAGGCCCGTGAGTGGATTTTGAGCAAGGGCGGAC
TGGGCAAAGTACGTGTGTTTACTCGTTATTGGCTGGCCCTTATCGGCGAATGGCCAT
GGGAACAGACCCCGAATCTGCCACCAGAAGTAATTTGGTTACCGGACTGGGCCCCG
TTCTCCATTTATAACTTCTCGCAATGGGCGCGCGCTACGATGATGCCACTGGCGGTA
CTGAGTGCGCGTCGGCCGAGTCGCCCCTTGCCGCCGGGCAACCGTTTGGATGCTTTA
TTTCCGGAAGGTCGCGAACATTTTGATTATTCTCTTCCTGTTAGAGATGGCGCGGGTT
GGGGAGATCGTTTCTTCCGCGCTGCGGACAAGGCACTCCATCGTTTACAGGACATGG
GTGCGCAGTATGGCCGCTTCGCACCGTGGCGCGATGCAGCGGTTCGCCATACCTTGG
AGTGGATTTTGCGCCATCAGGACGCGGATGGCGGATGGGGCGGAATACAACCGCCG
TGGGTATATGGACTGATGGCGCTGCACGTTGAAGGGTATGCGCTGGACCATCCTGTA
ATGGCGAAAGGCCTCGAAGCGCTCGACCATCCGGGTTGGCGAGTGGATAAAGGTGA
GGCCTCATGGATTCAAGCGTCCAACAGCCCTGTATGGGATACGATGCTTACGCTGAT
CGCGTTTGATGATACTGGCCTAGCCGAAGCGCATCCTGAAGCGACGGCCAAAGCGG
TGCAGTGGCTTCTTGATCACCAGATTCGGCGTCCGGGTGACTGGTCGCGCAAATTGC
CGGGAGTAAAACCGGGTGGATGGGCGTTCGAGTATGCAAACTCCCAGTACCCGGAT
ATAGATGATACTGCCGAAGCGCTTATCGCGCTGGCGCCCTTTCGTCACGATCCTGTA
TGGCAGACGAGAGGCATTGAGGAAGCAATAACCCTGGGCGTTGATTGGCTGATAGG
TATGCAATCTGCATCGGGTGGATGGGGCGCCTTCGATAAGGATAACAATAAACAAC
TGCTCACGAAAATTCCGTTTTGCGATTTCGGTGAAGCGCTAGATCCGCCAAGCGTGG
ATGTGACGGCCCATATTGTGGAAGCGCTGGCACGCCTGGGGCTCTCTGCGGAGCAC
CCAGCTCTGGCGAGAGCCCTGGCGTTCATCCGAGCAGAGCAGGAAGCCGATGGCCC
CTGGTTCGGCCGTTGGGGTGTTAATTATATCTATGGCACTTGCGCCGTGCTCCCGGCC
TTAGCGACGATCGGCGAAGATATGAACCAGCCCTATGTTGGCCGGGCCTGTGATTGG
CTGGTCAGTCGTCAGCAAGATAATGGTGGTTGGGGTGAGTCTTGCGCTTCGTATATG
GACCCAGCCCAGGCGGGATATGGTCCCGTTACTGCGTCGCAGACCGCGTGGGCGCT
GATGGCTTTGCTGGCGGTTAACCGCCCTGAAGATCGCGCAGCAATCGAACGCGGAT
GTCGGTACCTGGTCGAGCACCAGGAAAACGGTTGTTGGGAAGAGCCGCACTATACC
GCAACCGGTTTCCCGGGCTACGGTGTGGGTCAGAGCATAAAGCTGACCGATCCTGA
AATTGCTCGTCGGCTTATGCAAGGCAGTGAATTGTCGCGTGCTTTCATGCTTCGATAT
GACTTATATCGACATTATTTTCCGATGATGGCTCTGGGTCGCGCAGTACGTAGCGGC
CGTGTGAGCGGTGCA
SEQ ID NO: 9 PfuSHC1 AA Organism: Phaeospirillum fulvum
MTNQRTRLKVASERADEIDTDGDRTATLPVSPVVNSGKNSAPIGLAANTDSSLTVKIKA
AINAAGDWLLDRQNEDGHWVGPLQSNACMEAQWCLALWFLGLENHPLRPRLGQSLLE
TQREDGSWEVYYAASAGDINTTVEAYAALRSLGFPESDPRLTKAREWILSKGGLGKVR
VFTRYWLALIGEWPWEQTPNLPPEVIWLPDWAPFSIYNFSQWARATMMPLAVLSARRP
SRPLPPGNRLDALFPEGREHFDYSLPVRDGAGWGDRFFRAADKALHRLQDMGAQYGRF
APWRDAAVRHTLEWILRHQDADGGWGGIQPPWVYGLMALHVEGYALDHPVMAKGLE
ALDHPGWRVDKGEASWIQASNSPVWDTMLTLIAFDDTGLAEAHPEATAKAVQWLLDH
QIRRPGDWSRKLPGVKPGGWAFEYANSQYPDIDDTAEALIALAPFRHDPVWQTRGIEEA
ITLGVDWLIGMQSASGGWGAFDKDNNKQLLTKIPFCDFGEALDPPSVDVTAHIVEALAR
LGLSAEHPALARALAFIRAEQEADGPWFGRWGVNYIYGTCAVLPALATIGEDMNQPYV
GRACDWLVSRQQDNGGWGESCASYMDPAQAGYGPVTASQTAWALMALLAVNRPED
RAAIERGCRYLVEHQENGCWEEPHYTATGFPGYGVGQSIKLTDPEIARRLMQGSELSRA
FMLRYDLYRHYFPMMALGRAVRSGRVSGA
SEQ ID NO: 10 KdiSHC1 NT Organism: Komagataeibacter diospyri
ATGACCCGAGAGAGTCGCCCGTTGACCAAAACCGCGTCGAGTTCTGATGCTGTGATT
CACGATATTGCTTCAGGCACCACCTTTCCGGCGGCCGGCATCCAGTCCGTCAAGGGG
AAAGACAAGAGTTTGCCGGCTGCCAGCGCTCTGCGCACCATGGACAACTCGTTGAG
CCATGCCATCAGTAGCGCGTGCGATTGGCTGGTAGGCCAACAGAAACCGGATGGTC
ACTGGGTGGGCCCGGTTGCAAGTAACGCGAGCATGGAAGCGGAATGGTGCTTAGCG
CTCTGGTTCTTGGGCCTCGAAGATCACCCGCTGCGCCCGCGCCTGGGCAAAGCCTTA
CTGGAGATGCAGCGCGAAGATGGGAGCTGGGGTACGTATTATGGTGCTGGCAACGG
TGATATTAACGCGACCGTTGAGTCGTATGCGGCGCTGAGGTCCCTTGGCTATCCAGC
TGATGATCCGGCCGTGTCGCGCGCGGCGACCTGGATCGCGAGCAAAGGCGGATTGA
AGAATATTCGCGTGTTCACGCGTTATTGGCTGGCGTTAATTGGTGAGTGGCCGTGGG
AGAAAACGCCGAACTTACCGCCGGAAGTCATTTGGTTCCCCAATAAATTCGTATTCT
CGATATATAACTTTGCGCAGTGGGCGCGTGCCACCCTGGTTCCGTTGGCCATTCTGT
CAGCGCGGCGCCCTTCGCGCCCATTGCGACCGCAGGATCGTTTAGATGCCTTATTTC
CGGGTGGCCGCGCTAACTTCGACTATGAACTGCCAGCGCGTGGCGAACGCGATCTG
TGGGATCGCTTCTTCCGTACCACCGATCGTGGCTTGCACTGGCTCCAATCCCGCTTCT
TGAAACGCAACACATTGCGCGAGGCAGCAATCCGTCACATGCTGGAATGGGTGATT
CGTCATCAGGATGCCGACGGTGGATGGGGCGGCATCCAGCCGCCATGGGTTTATGG
TCTGATGGCACTGCACGGCGAGGATTACCAGTTCCATCACCCGGTTATGGCGAAAGC
CCTAAGTGCACTGAACGACCCCGGCTGGCGTCACGACCGTGGTGACGCGTCCTGGA
TACAAGCAACCAATTCTCCAGTCTGGGACACCATGCTGGCTATCATGGCCCTGCATG
ACGCCGACGGCGAAACCCGTTTCTCACCGCAGATGGAGAAGGCTCTCGGCTGGCTG
CTCGATCGTCAGGTGCGAGTGAAAGGAGATTGGTCGATTAAACTGCCGGATGTGGA
ACCAGGTGGCTGGGCCTTCGAATACGCCAACGATCGATACCCCGACACTGATGACA
CGGCTGTAGCGCTGATCGCTTTAAGCAGCTGCCGCAACCGCGAGGAATGGAAGAAA
CGGGGTGTCGAAGATGCTATCCGCCGCGGGGTCAATTGGCTGATCGGTATGCAAAG
CGAATGTGGCGGTTGGGGTGCCTTCGATAAGGACAACAACCGCAGTATTCTCAGTA
AAATTCCGTTCTGCGACTTTGGCGAAGCACTGGACCCGCCGTCGGTAGATGTAACAG
CGCACGTACTGGAAGCGTTCGGGGTGCTGGGTCTGCCGCGTGATCTGCCCGCCATCC
AGCGGGGCCTGGCCTATATTCGTGCAGAACAGGAACCGGATGGCCCGTGGTTCGGA
CGTTGGGGAGTGAATTATTTATATGGTACCGGTGCTGTGCTCCCTGCTTTGGCAGCC
ATCGGCGAGGACATGACGCAACCGTATATCACGCGCGCGTGTGATTGGCTGGTTGC
GCATCAACAGGAGAACGGCGGCTGGGGAGAATCGTGTGCCAGCTACATGGAGTTGT
CCGCCGTAGGGCGCGGCCCGACCACCCCGAGTCAGACGGCGTGGGCGCTGATGGGT
CTGATTGCCGCGAATCGTCCTCAGGATTATGGTGCGATCGCCCGTGGGTGTCGCTAT
CTGATCGATCTGCAACAAGCCGATGGGTCCTGGCATGAGAAAGAATTTACTGGCAC
GGGTTTCCCCGGATACGGGGTTGGTCAGACGATCCGGTTAGATGATCCAGCCCTTTC
CAAAAGACTTCAGCAGGGAGCCGAACTAAGTCGTGCATTCATGCTGCGATACGATC
TCTACAGGCAGTTCTTCCCGATAATGGCACTAAGTCGCGCAAGTCGTCTGATGAAAT
TAGAAAAG
SEQ ID NO: 11 KdiSHC1 AA Organism: Komagataeibacter diospyri
MTRESRPLTKTASSSDAVIHDIASGTTFPAAGIQSVKGKDKSLPAASALRTMDNSLSHAIS
SACDWLVGQQKPDGHWVGPVASNASMEAEWCLALWFLGLEDHPLRPRLGKALLEMQ
REDGSWGTYYGAGNGDINATVESYAALRSLGYPADDPAVSRAATWIASKGGLKNIRVF
TRYWLALIGEWPWEKTPNLPPEVIWFPNKFVFSIYNFAQWARATLVPLAILSARRPSRPL
RPQDRLDALFPGGRANFDYELPARGERDLWDRFFRTTDRGLHWLQSRFLKRNTLREAA
IRHMLEWVIRHQDADGGWGGIQPPWVYGLMALHGEDYQFHHPVMAKALSALNDPGW
RHDRGDASWIQATNSPVWDTMLAIMALHDADGETRFSPQMEKALGWLLDRQVRVKG
DWSIKLPDVEPGGWAFEYANDRYPDTDDTAVALIALSSCRNREEWKKRGVEDAIRRGV
NWLIGMQSECGGWGAFDKDNNRSILSKIPFCDFGEALDPPSVDVTAHVLEAFGVLGLPR
DLPAIQRGLAYIRAEQEPDGPWFGRWGVNYLYGTGAVLPALAAIGEDMTQPYITRACD
WLVAHQQENGGWGESCASYMELSAVGRGPTTPSQTAWALMGLIAANRPQDYGAIARG
CRYLIDLQQADGSWHEKEFTGTGFPGYGVGQTIRLDDPALSKRLQQGAELSRAFMLRY
DLYRQFFPIMALSRASRLMKLEK
SEQ ID NO: 12 AorSHC1 NT Organism: Acetobacter orleanensis
ATGACCACCCCATTGTTTAAAGGAATGGGCAACTCGCTGACCCACACCGTCAGCTCT
GCGTGCGAATGGCTGATTTCTCAACAAAATCCGGACGGTCATTGGGTGGGACCTGTC
GGAAGTAACGCTTCCATGGAAGCTGAATGGTGCCTGGCTCTTTGGTTTCTTGGCCTA
GAGGACCATCCACTGCGACCGAGACTCGGGAACGCGTTGTTACAAACTCAGCGTGA
AGATGGTTCATGGGACGTCTACCTGGGCGCAGGCAATGGTGACATTAATGCCACGG
TCGAAGCCTATGCTGCGTTACGGAGCCTGGGCTATCCGGAGAACACCCCGGCCCTTC
AGAAAGCGGCTACCTGGATCAAACAGAAAGGTGGGCTGAAGAATATCCGGGTGTTT
ACACGTTACTGGCTGGCGCTGATCGGCGAATGGCCATGGGAAAAGACGCCGAATCT
TCCGCCGGAAATCATCTGGTTCCCGAACAAATTTGTTTTCAGCATCTATAACTTCGCC
CAGTGGGCCCGTGCGACCCTGGTTCCGCTGGCGATTTTATCCGCCCGTCGTCCGTCA
CGCCCACTGCGTCCTCAGGACCGCCTAAACGCCCTGTTTCCAGAGGGACGCGGTAAC
TTTGATTATACGTTACCGAAGAAGGAAGGCGTGGATCTGTGGTCGGACTTCTTTCGT
ACAACCGACAAAGGCTTGCACTGGTTGCAATCCAAATTCCTTAAACGGAACACGAT
GCGTGAGGCAGCCATCCGTCACATGTTAGAATGGATAATCCGCCACCAGGATGCAG
ATGGCGGCTGGGGCGGTATCCAGCCACCCTGGGTTTATGGCCTTATGGCCCTGCACG
GCGAAGGATACCAGTTTCATCACCCGGTGATGGCAAAAGGTCTGGACGCACTGAAC
GATCCAGGCTGGCGGCACGACAAAGGTAACGCTAGCTGGATCCAAGCTACGAATAG
TCCTGTCTGGGATACGATGCTGGCAATTATGGCACTGCACGACGCCAAAGCGGAAG
ATCGCTTCACACCGCAGGTTGACAAAGCCCTGGGGTGGCTGCTTGATCGCCAAGTTC
GTGTGAAAGGGGATTGGAGCATTAAGTTGCCTGATGTCGAACCTGGCGGTTGGGCCT
TCGAATATGCAAACGACTTCTATCCTGACACTGATGACACGGCGGTGGCACTTATTG
CGCTGGCGAGTTGCCGGCATCGTCCGGAATGGCAAGAACGTGGCGTAGAGGATGCG
ATTGCTCGCGCCGTACGTTGGCTGGTGGCCATGCAGTCGTCTTGTGGTGGCTGGGGA
GCCTTCGATAAAGATAACAATAAGGCGTTGTTGAGTAAAATACCCTTTTGTGATTTC
GGCGAAGCACTCGATCCACCTTCAGTTGACGTAACGGCACATATCCTGGAAGCTTTC
GGTTTGCTGGGCCTTCCGCGCGATCTGCCGTGTATCAAACATGCGCTGGATTACGTG
CGCGCTGAGCAAGATCCTCAAGGCCCTTGGTTTGGTCGCTGGGGTGTTAATTATGTA
TATGGCACGGGCGCGGTGCTGCCTGCGTTAGCTGCAATCGGTGAAGATATGACTCAA
CCGTATATTACCAAAGCGTGCGATTGGTTAGTTGCGCATCAGCAGGAAGATGGCGG
CTGGGGTGAATCTTGCGCCAGTTACATGGACGCCTCAACCATTGGTAGAGGCAAAA
CCACACCGTCACAAACCGCATGGGCTCTGATGGGACTGATCGCAGCCGCGCGTCCG
CAGGACTATCCGGCCATTGAAAAGGGATGTCGCTACCTCATCGATCGTCAGGAACCT
GATGGCTCCTGGACGGAAGAAGATTATACCGGCACCGGCTTTCCGGGGTATGGCGT
GGGTCAAACGATTCGTCTGGACGATCCTGCCCTGTCCAAACGTTTACAGCAGGGTGC
TGAACTGTCCCGCGCCTTTATGTTGCGCTATGATTTATATCGCCAGTTCTTTCCGATT
ATGGCGCTCTCTCGCGCGTCAAGGCTGATTAGTCCGGAAACCGCCACCGAACAAGC
TGTAGAGGCGGCAGCGAAGAACCTGGAGAAAATCATTGCT
SEQ ID NO: 13 AorSHC1 AA Organism: Acetobacter orleanensis
MTTPLFKGMGNSLTHTVSSACEWLISQQNPDGHWVGPVGSNASMEAEWCLALWFLGL
EDHPLRPRLGNALLQTQREDGSWDVYLGAGNGDINATVEAYAALRSLGYPENTPALQK
AATWIKQKGGLKNIRVFTRYWLALIGEWPWEKTPNLPPEIIWFPNKFVFSIYNFAQWAR
ATLVPLAILSARRPSRPLRPQDRLNALFPEGRGNFDYTLPKKEGVDLWSDFFRTTDKGLH
WLQSKFLKRNTMREAAIRHMLEWIIRHQDADGGWGGIQPPWVYGLMALHGEGYQFHH
PVMAKGLDALNDPGWRHDKGNASWIQATNSPVWDTMLAIMALHDAKAEDRFTPQVD
KALGWLLDRQVRVKGDWSIKLPDVEPGGWAFEYANDFYPDTDDTAVALIALASCRHR
PEWQERGVEDAIARAVRWLVAMQSSCGGWGAFDKDNNKALLSKIPFCDFGEALDPPSV
DVTAHILEAFGLLGLPRDLPCIKHALDYVRAEQDPQGPWFGRWGVNYVYGTGAVLPAL
AAIGEDMTQPYITKACDWLVAHQQEDGGWGESCASYMDASTIGRGKTTPSQTAWALM
GLIAAARPQDYPAIEKGCRYLIDRQEPDGSWTEEDYTGTGFPGYGVGQTIRLDDPALSKR
LQQGAELSRAFMLRYDLYRQFFPIMALSRASRLISPETATEQAVEAAAKNLEKIIA
SEQ ID NO: 14 KrhSHC1 NT Organism: Komagataeibacter rhaeticus
ATGACCTTTCTGACTGGTACCGAGAAAATGAACAAAGAAAGTCGCCCGTTCCCGCC
GGGTACGACCGGCCCAGTTAGTGGTAAAGCGCCTCAGGCGACGGACGGTTCTTTCC
GCGCGCTAGATAATTCACTGTCTCACGCGTTATCCGCGGCCTGCGATTGGCTGATTG
GCCAGCAGAAACCGGATGGTCATTGGGTGGGCCCGGTCGCTAGCAACGCCAGTATG
GAAGCCGAGTGGTGCCTCGCCCTGTGGTTCTTGGGTCTGGATGATCATCCGCTACGC
CCGCGCCTCGGGCACGCGCTACTCGAAATGCAGCGTGAAGATGGATCGTGGGGCAT
CTACTACGGCGCGGGCAATGGAGATATTAATGCGACCGTTGAATCTTACGCGGCTCT
TCGCTCGCTTGGCTATGCAGCGGACGAACCGGCCCTAGCGAAAGCCGCAACTTGGA
TTGCAAGCAAAGGTGGTTTGCGTAACATCCGCGTATTCACTCGCTACTGGCTGGCAC
TGATCGGTGAATGGCCGTGGGAGAAAACCCCGAATCTGCCGCCTGAGGTTATTTGGT
TTCCTAATAAATTCGTTTTCAGCATTTACAACTTTGCCCAATGGGCCCGGGCAACCTT
AGTGCCCCTAGCTATCCTTTCAGCTCGCCGCCCGGCACGCCCATTACGCCCGCAAGA
CCGCTTGGATGCGCTGTTCCCAGGTGGCCGCGCGAATTTTGATTATGAACTTCCGCG
AAAAGAAGGTCGTGACCTTTGGGCTAGCTTCTTCCGCACCACCGACCGTGGCCTGCA
CTGGTTACAGAGCCGCGTTATGAAGAAGTCGAGTCTGAGAGAAGCTGCAATCCGTC
ACATGCTCGAGTGGATAATCCGTCATCAGGACGCCGACGGCGGCTGGGGCGGTATT
CAACCGCCTTGGGTCTATGGCTTAATGGCGTTACACGGTGAGGATTATCAGTTTCAT
CACCCAGTGATGGCAAAAGGCCTGGCGGCGTTAGACGATCCGGGATGGCGCCACGA
CCGCGGGCAGGCGTCCTGGATTCAGGCCACCAACAGTCCGGTATGGGACACGATGC
TTGCAATCATGGCGCTTCATGATGCCGATGGCGAAACCCGTTTCACCCCAGAAATGG
ATCGCGCACTGGGCTGGTTGCTGGATCGTCAGGTCCGCGTGAAGGGTGACTGGTCAA
TTAAGCTGCCGGATGTCGAACCAGGTGGCTGGGCGTTTGAGTATGCAAACGACCGA
TACCCGGATACTGATGATACAGCGGTGGCCCTTATTGCGCTGTCGTCGTGCCGTAAT
CGTCCAGAGTGGCAGGCACGCGGGGTAGAACCGGCAATTAAACGTGGGGTAAATTG
GCTTGTGGCCATGCAGTCAGAGTCAGGTGGCTGGGGCGCGTTTGATAAAGATAACA
ATCGCTCTTTGTTGGCTAAAATTCCGTTCTGCGATTTTGGCGAAGCCCTGGATCCGCC
TAGCGTGGACGTTACGGCCCATGTGCTAGAAGCATTCGGTGTGTTAGGCTTACCGCG
CGACATGCCAGCGATTCGCCGTGGCCTGGCCTATATTCGTGCGGAACAGGCTGCGG
ACGGGCCTTGGTTTGGTCGTTGGGGTGTTAACTATCTGTACGGTACCGGTGCCGTCC
TGCCGGCCCTTGCCGCTATCGGCGAGGATATGACGCAGCCGTATATTACGCGTGCTT
GCGATTGGCTTGTAGGATGTCAACAAGCAGATGGCGGGTGGGGAGAAAGCTGTGCC
TCATATATGGAGATTGCGGCGATTGGTCATGGGCCTACAACACCATCACAGACTGCA
TGGGCACTTATGGGTCTGATTGCGGCTCATCGTCCTCAAGATCACGCCGCTATCGCT
CGCGGCTGCCGTTACCTAATTGACCTTCAGCAACCGGATGGCCGCTGGGATGAAAA
GGAGTTTACGGGTACCGGATTCCCGGGTTACGGTGTCGGTCAAACCATTCGGTTAGA
CGATCCGGCCCTGTCGCAGCGCTTACAGCAAGGCGCCGAGCTGTCTCGTGCTTTTAT
GCTGCGTTACGATCTCTATCGTCAATTCTTCCCGATCATGGCTCTGTCCCGGGCCGCA
CGCGTGCTCAAAGCGGCTGGA
SEQ ID NO: 15 KrhSHC1 AA Organism: Komagataeibacter rhaeticus
MTFLTGTEKMNKESRPFPPGTTGPVSGKAPQATDGSFRALDNSLSHALSAACD
WLIGQQKPDGHWVGPVASNASMEAEWCLALWFLGLDDHPLRPRLGHALLEMQREDGS
WGIYYGAGNGDINATVESYAALRSLGYAADEPALAKAATWIASKGGLRNIRVFTRYWL
ALIGEWPWEKTPNLPPEVIWFPNKFVFSIYNFAQWARATLVPLAILSARRPARPLRPQDR
LDALFPGGRANFDYELPRKEGRDLWASFFRTTDRGLHWLQSRVMKKSSLREAAIRHML
EWIIRHQDADGGWGGIQPPWVYGLMALHGEDYQFHHPVMAKGLAALDDPGWRHDRG
QASWIQATNSPVWDTMLAIMALHDADGETRFTPEMDRALGWLLDRQVRVKGDWSIKL
PDVEPGGWAFEYANDRYPDTDDTAVALIALSSCRNRPEWQARGVEPAIKRGVNWLVA
MQSESGGWGAFDKDNNRSLLAKIPFCDFGEALDPPSVDVTAHVLEAFGVLGLPRDMPAI
RRGLAYIRAEQAADGPWFGRWGVNYLYGTGAVLPALAAIGEDMTQPYITRACDWLVG
CQQADGGWGESCASYMEIAAIGHGPTTPSQTAWALMGLIAAHRPQDHAAIARGCRYLI
DLQQPDGRWDEKEFTGTGFPGYGVGQTIRLDDPALSQRLQQGAELSRAFMLRYDLYRQ
FFPIMALSRAARVLKAAG
SEQ ID NO: 16 AmaSHC1 NT Organism: Acetobacter malorum
ATGGGCAACAGCCTGACGCACACTGTTTCAAGTGCGTGTGAGTGGCTGATAAGCCA
ACAGAAACCCGACGGTCACTGGGTAGGGCCTGTGGGATCTAACGCGAGCATGGAAG
CAGAATGGTGCCTTGCGCTATGGTTCCTGGGCCTGGATGATCACCCCTTGCGTCCGC
GCTTGGGCAACGCGTTGTTGCAGACGCAGCGTGATGATGGATCATGGGGTATCTACC
TCGGCGCAGGTAATGGTGATATTAATGCCACCGTTGAAGCGTATGCGGCCCTGCGTT
CTCTGGGTTATCCCGAAGATCATCCGGCTTTACATAAAGCATCTGCCTGGATTGCCC
AGAAAGGCGGCCTGCGCAACATCCGCGTCTTTACTCGTTACTGGCTTGCGCTGATTG
GCGAATGGCCCTGGGAAAAGACTCCGAACTTGCCGCCAGAAATCATCTGGTTTCCG
AATAAATTCGTCTTTAGCATATACAACTTCGCGCAGTGGGCACGCGCAACCCTGGTG
CCCTTGGCTGTGATCAGCGCACGTCGCCCATCCCGCCCGCTGAGGCCACAGGATAGG
CTGAACGCACTGTTTCCCGAGGGTCGCGCCAACTTCGATTATACCCTGCCGAAGAAG
GAAGGCAGAGACTTATGGAGTGACTTCTTTCGTACGACGGACAAGGGTCTGCACTG
GCTGCAAAGCAAGTTTCTGAAACGTAATACCTTACGTGAGGCAGCAATTCGCCACAT
GTTAGAATGGATTATTCGTCATCAGGATGCGGACGGTGGGTGGGGGGGGATTCAAC
CGCCTTGGGTGTACGGCCTTATGGCCCTGCATGGCGAAGGTTTCCAGTTCCATCATC
CTGTAATGGCTAAAGCGCTGGACGCGCTGAACGATCCGGGCTGGCGCCACGATAAA
GGTGACGCGAGCTGGATTCAAGCAACAAATAGCCCCGTATGGGATACCATGCTGGC
AATTATGGCCCTGCATGATGCCAAAGCAGAGGAACGCTTCACCCCGCAGATGGATA
AAGCGCTGGGTTGGTTGCTCGATCGCCAGGTTCGCGTCAAAGGCGATTGGAGCATCA
AACTGCCGGATGTGGAACCCGGTGGCTGGGCTTTCGAATATGCGAACGATTTCTACC
CGGATACAGACGATACGGCAGTCGCACTGATTGCGCTAGCATCCTGTCGCCACCGTC
CGGAGTGGAAAGAACGTGGAGTTGAAGAGGCGATCGCGCGGGCCGTCCGGTGGCTG
GTAGGCATGCAATCGAGTTGCGGTGGCTGGGGTGCCTTTGATAAAGACAATAATAA
AACTCTTCTGTCGAAGATTCCTTTCTGTGATTTCGGCGAAGCTTTAGACCCGCCAAGC
GTTGATGTTACCGCTCATATCCTTGAGGCTTTTGGTATTTTAGGACTGCCTCGTGACC
TGCCGTGCATTCAGCGCGCGCTTGCCTATGTCCGCGCCGAACAAGATCCACAGGGCC
CGTGGTTCGGTCGTTGGGGCGTGAATTATGTGTACGGGACCGGTGCTGTGCTGCCGG
CCCTGGCTGCAATCGGTGAGGACATGACCCAGCCCTACATCACTAAAGCCTGTGACT
GGCTGGCTGCCCATCAGCAGGATGACGGCGGTTGGGGCGAATCTTGCGCTTCATATA
TGGAAGTTTCCGCCATTGGTCGGGGTAAGACTACCCCAAGTCAGACCGCCTGGGCA
CTGATGGGTTTAATTGCCGCGGCCCGTCCGCAAGATTTCGCGGCGATCGAGAAAGG
GTGTCGTTATCTGATTGATCGTCAAGAACCGGATGGCTCGTGGACTGAAGAGGAATA
CACCGGTACCGGCTTTCCGGGTTATGGTGTGGGGCAAACCATTAAACTCGACGATCC
GACGCTGAGTAAGCGCTTATTGCAAGGTGCCGAGCTTAGCCGCGCCTTCATGCTCCG
CTACGACCTGTACCGTCAATTCTTTCCGATCATGGCTCTGTCCCGCGCGTCCCGTCTT
ATTACACCGAATGCGACTGCGGAGAAAGCAATCAACGCGGCGCGTAAGAATCTGGC
GGAAACCGTTGCG
SEQ ID NO: 17 AmaSHC1 AA Organism: Acetobacter malorum
MGNSLTHTVSSACEWLISQQKPDGHWVGPVGSNASMEAEWCLALWFLGLDDHPLRPR
LGNALLQTQRDDGSWGIYLGAGNGDINATVEAYAALRSLGYPEDHPALHKASAWIAQK
GGLRNIRVFTRYWLALIGEWPWEKTPNLPPEIIWFPNKFVFSIYNFAQWARATLVPLAVI
SARRPSRPLRPQDRLNALFPEGRANFDYTLPKKEGRDLWSDFFRTTDKGLHWLQSKFLK
RNTLREAAIRHMLEWIIRHQDADGGWGGIQPPWVYGLMALHGEGFQFHHPVMAKALD
ALNDPGWRHDKGDASWIQATNSPVWDTMLAIMALHDAKAEERFTPQMDKALGWLLD
RQVRVKGDWSIKLPDVEPGGWAFEYANDFYPDTDDTAVALIALASCRHRPEWKERGVE
EAIARAVRWLVGMQSSCGGWGAFDKDNNKTLLSKIPFCDFGEALDPPSVDVTAHILEAF
GILGLPRDLPCIQRALAYVRAEQDPQGPWFGRWGVNYVYGTGAVLPALAAIGEDMTQP
YITKACDWLAAHQQDDGGWGESCASYMEVSAIGRGKTTPSQTAWALMGLIAAARPQD
FAAIEKGCRYLIDRQEPDGSWTEEEYTGTGFPGYGVGQTIKLDDPTLSKRLLQGAELSRA
FMLRYDLYRQFFPIMALSRASRLITPNATAEKAINAARKNLAETVA
SEQ ID NO: 18 RacSHC1 NT Organism: Rhodoblastus acidophilus
ATGGGCGAGGCGCTCACAGTCGCGGATACCCCGGTTTCACAAGACAACTTTGCCGC
CGCTTTGCGCTCCGGCGTGCGCGCCGCAGTTGATTGGCTGGTTGAGCGCCAGAAACC
AGACGGTCATTGGGTCGGCCGCGCCGAAAGTAACAGTTGTATGGAAGCGCAGTGGC
GCCTGGCACTGTGGTTCCTCGGCTTGGACGATCACCCGTTATGTGCCCGCCTGGCGC
AAAGCCTGTTGGACACCCAAAGACCTGATGGAGCTTGGGAAATTTATTACGGCGCG
CCGAATGGGGACATCAATACAACAGTCGAAGCTTATGCCGCGTTACGGTGCTCCGG
ATATGCGGCGGGTCACCCAGCATTATTGAATGCCCAGGCCTGGATCGAGAGTAAGG
GTGGACTGAAGAACATTCGTGTGTTTACGCGCTATTGGCTGGCGCTGATTGGTGAGT
GGCCGTGGGAAAAGACCCCTAACGTACCGCCTGAAGTCATCTGGTTTCCGCTCTGGT
TTCCGTTCAATATTTATCATTTCGCTCAATGGGCACGGGCTACACTTGTTCCCATTAC
CGTTTTGTCGGCACGGCGTCCGTCCCGTCCGCTGCCGCCGCAGAACCGGCTGGATGC
ACTCTTTCCGGAAGGCCGTGACGCGTTTGATTACGCGCTTCCGGCGAAACCGCAGCC
GGATTTGTGGGACCGCTTCTTTCGTCGCGCTGACAAAATCCTGCATGGCTTACAAGA
TTTCGGTCATCTTACAGGTTTAGGTGGTTTGCGTTCAGCAGCGGTGCGCCATGTATTG
GAATGGATCGTCAAACACCAGGACGCCGACGGGGCCTGGGGTGGCATCCAGCCGCC
GTGGATTTATAGCCTGATGGCCTTGAAAACGGAAGGCTATCCTGTTACGCACCCAGT
TCTGGCCAAAGGGCTAGATGCACTGAACGATCCGGGATGGCGCGTCGATGTGGGTG
ATGCAACGTTCATCCAAGCGACCAACTCCCCTGTCTGGGATACGATGTTGACTCTTC
TGGCGTTTGACGACGCTGAAGCTCTGGACTCGGAAGCCGCCGATAAAGCGGTTGAC
TGGCTGCTGCGCCGTCAAGTGCGGGTTCCGGGCGATTGGTCCGTCAAACTGCCTAAA
GTTCAGCCTGGCGGCTGGGCTTTTGAATATGCTAACAACTGCTACCCGGATACCGAC
GACACAGCGGTGGCATTGATAGCCCTAGTTCCGTTTCGTACCCAGGAAAAGTGGAA
GGCGCGTGGCATTGAAGAAGCGATTCAGTTAGGCGTGGATTGGCTGATCGCGATGC
AGTCTCAGTCGGGCGGCTGGGGTGCCTTTGACAAAGATAATACCCGGAAAATTCTTA
CCAAAATACCGTTTTGCGATTTCGGCGAAGCTCTTGATCCGCCGAGTGTCGATGTGA
CGGCACACGTGGTGGAAGCGTTTGGCAAACTGGGTATATCCCGGGAGCACCCGAGT
ATGATTAGAGCGTTACGCTACATTCGTGAACAACAGGAAGCGGATGGACCGTGGTT
TGGCCGCTGGGGAGTCAACTACCTGTATGGGACGGGCGCGGTGTTGCCAGCGCTTGC
AGCAATCGGCGAGGATATGACCCAGCCGTATATTGGCCGCGCCTGCGATTGGCTGGT
TAGTCGCCAGCAGGCCAATGGTGGCTGGGGTGAAAGCTGTGCGTCATATATGCAAG
CAGATCAGGCGGGGCGGGGCGAAGCAACTGCATCGCAGACAGGATGGGCGTTGATG
GCCCTGCTGGCGGTGGGCCGTGCCCAAGATCGCGAAAGCATCGAACGCGGTTGCCA
ATATCTTCTGGAAAATCAGCGTGAGGGCACCTGGCCTGAGCCTCACTTTACCGGTAC
CGGCTTCCCGGGCTACGGTGTGGGCCAAACTATCCGTCTGGATGACCCACTCTTGGC
ACAGAGGTTGAAGCAGGGCCCCGAGTTGAGCCGCGCGTTCATGCTGCGCTACGGCA
TGTATTGCCACTACTTCCCGCTGATGGCGTTATCCCGCGCCATTCGTCGCATC
SEQ ID NO: 19 RacSHC1 AA Organism: Rhodoblastus acidophilus
MGEALTVADTPVSQDNFAAALRSGVRAAVDWLVERQKPDGHWVGRAESNSCMEAQ
WRLALWFLGLDDHPLCARLAQSLLDTQRPDGAWEIYYGAPNGDINTTVEAYAALRCSG
YAAGHPALLNAQAWIESKGGLKNIRVFTRYWLALIGEWPWEKTPNVPPEVIWFPLWFPF
NIYHFAQWARATLVPITVLSARRPSRPLPPQNRLDALFPEGRDAFDYALPAKPQPDLWD
RFFRRADKILHGLQDFGHLTGLGGLRSAAVRHVLEWIVKHQDADGAWGGIQPPWIYSL
MALKTEGYPVTHPVLAKGLDALNDPGWRVDVGDATFIQATNSPVWDTMLTLLAFDDA
EALDSEAADKAVDWLLRRQVRVPGDWSVKLPKVQPGGWAFEYANNCYPDTDDTAVA
LIALVPFRTQEKWKARGIEEAIQLGVDWLIAMQSQSGGWGAFDKDNTRKILTKIPFCDF
GEALDPPSVDVTAHVVEAFGKLGISREHPSMIRALRYIREQQEADGPWFGRWGVNYLY
GTGAVLPALAAIGEDMTQPYIGRACDWLVSRQQANGGWGESCASYMQADQAGRGEA
TASQTGWALMALLAVGRAQDRESIERGCQYLLENQREGTWPEPHFTGTGFPGYGVGQT
IRLDDPLLAQRLKQGPELSRAFMLRYGMYCHYFPLMALSRAIRRI
SEQ ID NO: 20 AmeSHC1 NT Organism: Amphiplicatus metriothermophilus
ATGTTGGATGAGCAAGCAGCCAAAGCCGCCCGGGGCGATAATTTCCAAGCCGCGTT
GTCAGAGGCAATCCAGGCCTCGGCAGATTGGTTGGTCCGCCGGCAGAAACCGGAAG
GTTACTGGGTGGGCCGCCTGGAGTCAAATAGTTGTATGGAAGCGGAGTGGCGTTTG
GCGTTGTGGTTTATGGGGTTAGATGATCACCACTTGTGTCCGCGCCTGGCAGCGTCG
CTGCGGGAATCGCAGTCTGCGGATGGTAGCTGGCGTATTTATCACGGAGCGCCGGC
AGGGGATATTAATACCACCGTGGAAGCGTATGCGGCCCTGCGCTGTCACGGGGACG
ATCCGCACGCCCCTCACATGGCGAGGGCGCGCGCGTGGATCCTGTCAAAAGGTGGG
CTGCGTAATGTCCGCGTGTTCACGCGTTATTGGCTGGCGTTAATTGGTGAGTGGCCG
TGGGATCGTACCCCGAATCTCCCGCCGGAAGTCATTTGGTTCCCTAATTGGTTCGTTT
TCTCCATTTATAACTTTGCACAATGGGCTCGTGCTACACTTATGCCAATTGCGGTCCT
GTCTGCCCGCCGCCCGACCCGCCCTCTCCCGGCAGAACGGCGTCTAAATGAACTGTT
TCCGGAAGGTCGGGCTCGTTTTGATTACGAGCTTCCGCCTAAACCGGGCGCTGACTT
TTGGGATCGCTTCTTCCGTGCAGCTGATAAGACCCTGCACGCATTACAGGGCTTTGG
TCGGAAAACTGGCCTGTCCCTGGGGGGGGATGCGGCGATTAAACGCGCGCTGGAAT
GGATCATCCGGCATCAGGACGCAGATGGCGTCTGGGGTGGCATTCAGCCGCCATGG
ATCTATTCACTGATGGCACTTCATACCGAAGGGTATGCTTTAGATCATCCGGTGCTG
GCTAAAGGACTGGCGGCGCTGGATGATCCGCGGTGGAGAGTGGATGTAGGCGAAGC
GACCTGGATCCAAGCTTCGACGAGTCCGGTCTGGGACACGGAGCTTACTCTATTGGC
TTTACACGATGCGGATGTCGTCAAAGCCCACGAAGCCGCGGTTGAACGCGCGGTGC
AATGGCTCTTAGATCAACAGGTTAGACGTCCGGGCGATTGGCGTGTAAAAGTGCCG
AACGTGGAACCAGGCGGTTGGGCATTTGAATACAAGAATTACTTCTACCCGGATACC
GATGATACCGCCGTCGCCCTGATTGCTCTCGCACCGTTTCGTGATGATCCGAAATGG
AAGGCGAAAGGCTTGGACGAGGCGATTCGTCTTGGGGTTGATTGGCTGATCGGTAT
GCAGTCTGAATGCGGCGGTTGGGGCGCCTTTGACAAAGACAATGATAAGAAAATTC
TTACCAAGATCCCCTTTTGTGATTTCGGCGAGGCGCTCGATCCACCAAGTGTAGACG
TGACCGCCCATGTGATTGAAGCGTTTGGTAAACTGGGCTACGACAAAACACACCCG
GCGATGAAACGCGCCCTGGATTGGATGAAGAAGGAGCAGGAACCGGATGGTTCTTG
GTTTGGCCGCTGGGGTGTGAACTATGTTTATGGTACTGGGGCAGCATTACCGGCTCT
GGAAGCTATCGGCGAAGATATGTCTGCACCGTATGTACAGCGTGCTTGCGATTGGCT
GGTTTCAATGCAGCAAGCGGACGGTGGTTGGGGTGAATCTTGCGCGTCCTATATGGA
TCCTGCGATGAAAGGTCGTGGGACCCCTACTGCGAGCCAGACTGCATGGGCACTGA
TGGGGTTGGTTGCGGCCAACCGCGCGCAAGATCGTGAAGCCATACAGAAAGGTCTG
GCATTTCTAGTTGAACGCCAGAAGAACGGTACCTGGGAAGAGCCGGAATACACTGG
CACCGGTTTTCCAGGTTACGGTGTAGGCCAGACTATTAAACTCGGCGATCCGCTCCT
TGCAGAACGCCTTAAACAGGGTCCGGAATTAAGTCGCGCATTTATGATTAACTATAA
CCTCTATCGCCATTACTTTCCGCTTATGGCCATGGGACGCGCGCGCAGACTGCTTGG
TGCC
SEQ ID NO: 21 AmeSHC1 AA Organism: Amphiplicatus metriothermophilus
MLDEQAAKAARGDNFQAALSEAIQASADWLVRRQKPEGYWVGRLESNSCMEAEWRL
ALWFMGLDDHHLCPRLAASLRESQSADGSWRIYHGAPAGDINTTVEAYAALRCHGDDP
HAPHMARARAWILSKGGLRNVRVFTRYWLALIGEWPWDRTPNLPPEVIWFPNWFVFSI
YNFAQWARATLMPIAVLSARRPTRPLPAERRLNELFPEGRARFDYELPPKPGADFWDRF
FRAADKTLHALQGFGRKTGLSLGRDAAIKRALEWIIRHQDADGVWGGIQPPWIYSLMA
LHTEGYALDHPVLAKGLAALDDPRWRVDVGEATWIQASTSPVWDTELTLLALHDADV
VKAHEAAVERAVQWLLDQQVRRPGDWRVKVPNVEPGGWAFEYKNYFYPDTDDTAVA
LIALAPFRDDPKWKAKGLDEAIRLGVDWLIGMQSECGGWGAFDKDNDKKILTKIPFCDF
GEALDPPSVDVTAHVIEAFGKLGYDKTHPAMKRALDWMKKEQEPDGSWFGRWGVNY
VYGTGAALPALEAIGEDMSAPYVQRACDWLVSMQQADGGWGESCASYMDPAMKGR
GTPTASQTAWALMGLVAANRAQDREAIQKGLAFLVERQKNGTWEEPEYTGTGFPGYG
VGQTIKLGDPLLAERLKQGPELSRAFMINYNLYRHYFPLMAMGRARRLLGA
SEQ ID NO: 22 AdsSHC1 NT Organism: Acetobacter sp. DsW_54
ATGGGCAACTCCCTCGCTCACACCGTGGCCGCGGCATGTGATTGGCTTATCGGCGAG
CAGAAAGCAGACGGACATTGGGTTGGTCCAGTAGCGAGTAATGCCTCGATGGAAGC
AGAATGGTGTCTGGCCCTGTGGTACTTGGGCCTGGAAGATCACCCGTTGCGTCCGCG
CTTGGGAAAAGCCCTGTTACACATGCAACGAGAGGATGGTTCTTGGGGCACGTACT
GGGGTGCCGGCAATGGAGATATAAATGCGACTGTTGAAGCCTATGCCGCCTTGCGT
AGTCTGGGCTATGCAGCAGACACGCCGGAACTGTCAAAAGCTTGTGCGTGGATCAT
GCGCATGGGTGGTCTGCGTAATGTTCGTGTTTTCACCCGCTATTGGCTGGCGCTGATC
GGCGAATGGCCGTGGGAGCAGACCCCAAACTTACCGCCTGAAGTGATTTGGTTCCCT
AACAAATTCGTTTTCTCTATCTACAATTTTGCTCAATGGGCTCGCGCTACATTAGTAC
CACTGGCGATCCTGTCGGCTAGACGCCCGAGCCGTCCGCTGCGTCCTCAGGATCGCC
TGGATGCCCTGTTTCCACAAGGCCGTGAAAACTTCGACTATGTGCTGCCGAAGAAAG
AAGGCGTTGATCTGTGGTCGTCCTTCTTTCGCACTACTGATAAAGGTCTGCATTGGTT
GCAAAGCCGTTTCCTGAAACGCAACACAGTGCGTGAGGCTGCGATTCGCCACATGC
TTGAGTGGATCATTCGCCACCAAGATGCGGACGGCGGTTGGGGCGGCATCCAGCCG
CCGTGGGTATATGGCTTGATGGCCCTGCACGGCGAGGATTACCAATTTCATCATCCA
GTGATGGCAAAAGCGTTAGCGGCCCTGGATGACCCCGGTTGGCGTCGGGACCAGGG
TGATGCCTCTTGGGTTCAGGCGACAAACAGCCCGGTGTGGGATACGATGCTGGCCCT
TATGGCCCTGCACGATGCGAACGCTGAGGAACGCTACACTCCCCAGATGGATAAAG
CCCTCGATTGGCTGCTGGCCCGACAGGTGCGCGTTAAAGGTGATTGGAGTATTAAGT
TGCCGGATGTGGAACCTGGCGGCTGGGCATTTGAGTATGCTAATGATCGCTACCCAG
ACACGGATGATACCGCTGTAGCACTGATCGCCCTGTCCAGCTGCCGCAATAGAGAA
GAATGGAAAGAGAAAGGCGTAGAGGACGCGATTACCCGCGGTGTGAACTGGTTGAT
CGCGATGCAGAGCTCTTGTGGCGGTTGGGGTGCGTTTGATAAAGACAATAATCGTAG
CCTATTGAGCAAAATCCCCTTTTGTGACTTTGGGGAAGCCCTGGATCCGCCGTCGGT
CGATGTGACGGCCCATGTACTGGAAGCCTTTGGCCTGCTTGGCGTACCACGTCAGAC
TCCAGCGTTACAGCGAGGTCTGGCTTACATTCGCGCAGAACAGGAAGCTTCGGGCG
CATGGTTCGGTCGTTGGGGTGTGAACTATTTGTATGGAACTGGTGCCGTTCTGCCTG
CTCTCGCAGCCATTGGCGAAGATATGACTCAACCCTATATTACCAGAGCTTGCGACT
GGCTCATCGCGCATCAGCAGGAAGATGGCGGCTGGGGCGAATCTTGTGCCAGCTAT
ATGGACGTGTCCAGCATTGGCTGGGGCACGACGACGCCGTCCCAAACCGCATGGGC
CTTAATGGGTCTGATTGCGGCAAATCGTGAACAGGACCATCCGGCAATTGCCCGGG
GCTGCCGTTACCTGATCGATCGTCAAGAAACAGATGGTTCGTGGACGGAAGAAGAA
TTTACCGGCACGGGATTTCCAGGCTACGGTGTGGGCCAGACGATTAAGCTCGACGAC
CCGGCTGTCGCGAAGCGCTTGCAACAGGGAGCAGAGCTGAGCCGCGCGTTTATGTT
GCGCTATGATCTGTATCGCCAATTCTTCCCGCTGATGGCACTGAGCCGGGCGGCTCG
AATTATGCCGGTCGGGCAA
SEQ ID NO: 23 AdsSHC1 AA Organism: Acetobacter sp. DsW_54
MGNSLAHTVAAACDWLIGEQKADGHWVGPVASNASMEAEWCLALWYLGLEDHPLRP
RLGKALLHMQREDGSWGTYWGAGNGDINATVEAYAALRSLGYAADTPELSKACAWI
MRMGGLRNVRVFTRYWLALIGEWPWEQTPNLPPEVIWFPNKFVFSIYNFAQWARATLV
PLAILSARRPSRPLRPQDRLDALFPQGRENFDYVLPKKEGVDLWSSFFRTTDKGLHWLQ
SRFLKRNTVREAAIRHMLEWIIRHQDADGGWGGIQPPWVYGLMALHGEDYQFHHPVM
AKALAALDDPGWRRDQGDASWVQATNSPVWDTMLALMALHDANAEERYTPQMDKA
LDWLLARQVRVKGDWSIKLPDVEPGGWAFEYANDRYPDTDDTAVALIALSSCRNREE
WKEKGVEDAITRGVNWLIAMQSSCGGWGAFDKDNNRSLLSKIPFCDFGEALDPPSVDV
TAHVLEAFGLLGVPRQTPALQRGLAYIRAEQEASGAWFGRWGVNYLYGTGAVLPALA
AIGEDMTQPYITRACDWLIAHQQEDGGWGESCASYMDVSSIGWGTTTPSQTAWALMG
LIAANREQDHPAIARGCRYLIDRQETDGSWTEEEFTGTGFPGYGVGQTIKLDDPAVAKR
LQQGAELSRAFMLRYDLYRQFFPLMALSRAARIMPVGQ
SEQ ID NO: 24 RfaSHC1 NT Organism: Rhodopseudomonas faecalis
ATGACTACACATTCGACGATGGCGCAGCGGGCGGGCGCGTCTTGTAATATGGATGG
CGCCTTGCAGGCTACCATTCAGCAAGCAACTGAGTGGCTCATGAGTCAGCAGAAAC
CCGACGGCCATTGGGTGGGACGGAGCGAGAGTAATGCATGCATGGAAGCCCAATGG
TGTCTCGCCCTGTGGTTCATTGGTCTGGAAGATCATCCTCTGCGTGTTCGCCTGGGCC
AAGCCCTGCTCGATACTCAGCGGCCCGATGGCGCGTGGCATGTTTTCCACGGCGCGC
CGAACGGAGATATTAACGCCACGGTGGAAGCTTATGCCGCGCTTCGTTCTCTGGGTC
ACCGCGATGATGAACCTGCTCTGCGCAAAGCGCGGGAATGGATCCTGGCTAAGGGT
GGGCTGCGCAACATCCGCGTCTTTACCCGCTATTGGCTGGCGGTCATTGGGGAATGG
CCTTGGGACAAAACCCCTAATATTCCGCCCGAAGTCATTTGGCTACCCACTTGGTTC
CCGCTGTCAATTTACAACTTTGCCCAATGGGCGCGTGCGACCCTCATGCCGATCGCG
ATTCTGAGCGCCCACCGTCCATCCCGCGCATTGCCGCCGGAAAATCGCCTGGATGCA
CTGTTTCCGGAAGGTCGTGATAACTTCAATTATGATCTGCCGGAACGGCTGGGCGCG
GGCGTGTGGGACGCGTTCTTCCGCAAAGTTGACACAATCCTGCACTCGTTACAGACC
TGGGGTGCCAAACGTGGCCGTCATGGCTTAATGCGCCGCGCAGCATTAAATCATGTA
CTGGAGTGGATCATTCGTCACCAAGATGCCGACGGGGCGTGGGGCGGTATCCAACC
ACCATGGATCTATGGGATTATGGCCCTTCATTGCGAAGGTTACGCACTGAACCATCC
GGTCGTGGCGAAAGCCCTGGACTGTATGAATGATCCGGGCTGGCGTGTGGACGTCG
GTGACGCGAGTTTCCTGCAAGCCACCAACAGTCCGGTGTGGGACACCCTTCTGTCTT
TAATGTCCCTCGAGGACGCTGAGATGCGTGGTCAACATCCGGAAGAGGTAGAGCGC
TCGGTTCGCTGGGTGTTACAGCGCCAGGTCCGTGTGCCAGGCGATTGGTCGGTTAAA
CTGCCAAATGTAAAGCCTGGTGGCTGGGCCTTCGAATACGCAAACAATTTCTATCCT
GACACCGATGACACCGCGGTTGCGTTAATGGCCCTGGCGCCATTTCGGAATGATCCG
AAATGGCAAGCTGAAGGGATTGAAGAGGCCATTCAGTTAGGTGTCGATTGGCTCAT
AGGTATGCAGTGCAAAGGCGGTGGTTGGGGTGCGTTTGATAAGGATAATAATCAGA
AGCTGCTAGCTAAAATCCCCTTTTGTGATTTCGGTGAAGCCCTGGACCCACCTTCTGC
GGATGTAACCGCGCATGTCATCGAAGCGTTTTGCAAGTTAGGCATTGACCGCCGACA
TCCTTCAATGGCCCGAGCGGTTGCGTATTTGAAGCGCGAACAAGAACCCAACGGTC
CATGGTTCGGTCGTTGGGGCGTAAATTACCTGTATGGAACCGGGGCTGTGCTGCCGG
CTCTGGCTGCGATAGGCGAGGATATGTCCCAACCGTATATTGGCCGTGCATGCGATT
GGTTGGTGGCGCAACAGCAGGACTCAGGCGGCTGGGGCGAGTCATGTAGCTCTTAC
ATGGACCCGAGCGCAGCGGGTCGTGGTGTGGCCACGGCCAGTCAGACCGCCTGGGC
GGTGATGGCTTTACTGGCTGCCAATCGTCCGAAGGATCGTGACGCGATCGAGCGGG
GCTGTTTATTTCTTATCGATAATCAACGTCTGGGTACTTGGGTTGAACCCGAGTTTAC
CGGCACCGGCTTCCCTGGCTACGGGGTGGGGCAGACGATCAAATTAAACGATCCGC
TTCTGTCAAAACGCTTGATGCAGGGTCCTGAATTGTCTCGTAGTTTTATGCTCCGCTA
TGATATGTATCGCCATTATTTTCCGCTGACGGCCCTGGGGCGCATGAAAGCAATGCT
CGCAAAAGAAGCAGCCGCTACCCGT
SEQ ID NO: 25 RfaSHC1 AA Organism: Rhodopseudomonas faecalis
MTTHSTMAQRAGASCNMDGALQATIQQATEWLMSQQKPDGHWVGRSESNACMEAQ
WCLALWFIGLEDHPLRVRLGQALLDTQRPDGAWHVFHGAPNGDINATVEAYAALRSL
GHRDDEPALRKAREWILAKGGLRNIRVFTRYWLAVIGEWPWDKTPNIPPEVIWLPTWFP
LSIYNFAQWARATLMPIAILSAHRPSRALPPENRLDALFPEGRDNFNYDLPERLGAGVW
DAFFRKVDTILHSLQTWGAKRGRHGLMRRAALNHVLEWIIRHQDADGAWGGIQPPWIY
GIMALHCEGYALNHPVVAKALDCMNDPGWRVDVGDASFLQATNSPVWDTLLSLMSLE
DAEMRGQHPEEVERSVRWVLQRQVRVPGDWSVKLPNVKPGGWAFEYANNFYPDTDD
TAVALMALAPFRNDPKWQAEGIEEAIQLGVDWLIGMQCKGGGWGAFDKDNNQKLLA
KIPFCDFGEALDPPSADVTAHVIEAFCKLGIDRRHPSMARAVAYLKREQEPNGPWFGRW
GVNYLYGTGAVLPALAAIGEDMSQPYIGRACDWLVAQQQDSGGWGESCSSYMDPSAA
GRGVATASQTAWAVMALLAANRPKDRDAIERGCLFLIDNQRLGTWVEPEFTGTGFPGY
GVGQTIKLNDPLLSKRLMQGPELSRSFMLRYDMYRHYFPLTALGRMKAMLAKEAAAT
R
SEQ ID NO: 26 KmeSHC1 NT Organism: Komagataeibacter medellinensis
ATGACCTTCTTGACTGGTACCGAAAAGATGAATAAAGAAAGCCGTCCGTTCCCGCCC
ACCGCAAGCGGCACTGCGGGTCCGGCCGCGAGCACCACCCAGCAAGCACCTACGGG
CAGCCCGCGTGCTCTGGACAATTCATTATCTCATACGATCAGTGCGGCATGCGATTG
GCTGATAGGACAGCAGAAACCAGATGGGCATTGGGTGGGACCAGTGGCTAGCAATG
CCAGCATGGAAGCAGAATGGTGTCTGGCACTGTGGTTCCTGGGTCTGGAAGATCATC
CGTTACGACCGCGCTTGGGCCATGCTCTGTTAGAAATGCAGCGCGAGGATGGTAGTT
GGGGCATTTACTACGGCGCGGGTAACGGCGATATTAATGCCACAGTGGAAAGCTAT
GCCGCTCTGCGCTCTTTGGGTTATGGAGCTGATGAACCGACATTGGCGCGCGCGGCC
GAATGGATTGCATCCAAGGGCGGTCTGCGCAATATCCGTGTGTTTACCCGTTATTGG
CTGGCCTTGATCGGTGAATGGCCGTGGGAAAAGACACCTAATCTGCCGCCTGAAGT
GATTTGGTTTCCAAATTCGTTCGTCTTTAGCATCTACAATTTCGCACAGTGGGCACGC
GCGACCCTGGTACCGCTGGCCATCCTGAGTGCAAGACGTCCCGCGCGTCCGCTGCGC
CCGCAGGATCGCCTCGATGCCCTTTTCCCAGGTGGCCGTGCCAATTTTGACTACGAG
CTACCGCGTAAAGAAGGCCGCGATTTGTGGGCGTCATTCTTCCGTACCACCGATCGA
GGTCTGCATTGGTTACAATCACGTGTTCTCAAGAAGAACTCCGTTCGCGAAGCAGCA
ATTCGCCACATGCTGGAATGGATTATCCGCCATCAAGACGCAGATGGTGGTTGGGGT
GGTATACAGCCACCATGGGTTTATGGCTTGATGGCCTTACATGGTGAGGATTACCAG
TTTCATCACCCCGTAATGGCGAAAGCCCTGTCAGCTCTGAATGATCCCGGCTGGCGT
CACGACCGTGGCGATGCAAGTTGGATTCAAGCGACGAATAGCCCGGTGTGGGATAC
TATGCTGGCACTGATGGCGCTGCACGATGCGGACGGCGAAACGCGTTTCACCCCTCA
GATGGACAAAGCGATGGGGTGGTTACTGGATCGCCAGGTTCGAGTTAAGGGTGACT
GGAGCATTAAATTACCCGATGTTGAACCGGGTGGATGGGCATTTGAATACGCGAAC
GACCGGTATCCTGACACCGATGATACCGCGGTGGCGCTGATTGCCCTTTCGTCTTGT
CGTAACCGGCCGGAATGGCAGGCGCGAGGGGTCGAAGCAGCAATCAAGCGCGGCG
TGAACTGGTTAGTTGCAATGCAGTCAGAATCGGGTGGCTGGGGCGCGTTTGATAAG
GACAACAACCGCAGTCTGCTGGCGAAAATTCCGTTTTGCGACTTCGGCGAAGCATTG
GACCCGCCGTCTGTAGACGTAACTGCGCATGTGCTAGAAGCGTTCGGCCTCTTGGGC
TTGCCGCGCGATATGCCGGCTATTAGGCGCGGACTCGCCTACATCCGTGCGGAACAA
GCTGCGGAAGGACCGTGGTTTGGGCGCTGGGGTGTGAATTATTTGTACGGTACTGGT
GCTGTACTGCCGGCCTTAGCCGCAATTGGCGAAGATATGACCCAGCCATACATCGCG
AAAGCGTGCGATTGGCTGGTTGGTTGCCAGCAGGAAAACGGCGGTTGGGGTGAAAG
TTGCGCGAGCTATATGGAGATTTCGAGTATCGGTCGTGGCCCTACCACGCCGTCACA
AACGGCGTGGGCTTTGATGGGCCTGGTTGCAGCCAATCGTAGGCAGGATCACGATG
CGATTGTGCGCGGCTGTCGGTATCTGATCGATCTCCAGCAGCCGGATGGACGGTGGG
ACGAGAAAGAATTTACCGGCACTGGGTTTCCGGGCTATGGCGTGGGCCAGACCATT
CGTCTGGATGATCCTGCGCTTAGCAAACGCTTACAGCAGGGAGCCGAACTGAGCCG
CGCGTTCATGCTGCGATACGACCTGTACCGCCAGCTTTTCCCCATCATGGCACTGAG
CCGCGCGGCCAGGGTGCTGAAAGCCGCAGGC
SEQ ID NO: 27 KmeSHC1 AA Organism: Komagataeibacter medellinensis
MTFLTGTEKMNKESRPFPPTASGTAGPAASTTQQAPTGSPRALDNSLSHTISAACDWLIG
QQKPDGHWVGPVASNASMEAEWCLALWFLGLEDHPLRPRLGHALLEMQREDGSWGIY
YGAGNGDINATVESYAALRSLGYGADEPTLARAAEWIASKGGLRNIRVFTRYWLALIGE
WPWEKTPNLPPEVIWFPNSFVFSIYNFAQWARATLVPLAILSARRPARPLRPQDRLDALF
PGGRANFDYELPRKEGRDLWASFFRTTDRGLHWLQSRVLKKNSVREAAIRHMLEWIIR
HQDADGGWGGIQPPWVYGLMALHGEDYQFHHPVMAKALSALNDPGWRHDRGDASW
IQATNSPVWDTMLALMALHDADGETRFTPQMDKAMGWLLDRQVRVKGDWSIKLPDV
EPGGWAFEYANDRYPDTDDTAVALIALSSCRNRPEWQARGVEAAIKRGVNWLVAMQS
ESGGWGAFDKDNNRSLLAKIPFCDFGEALDPPSVDVTAHVLEAFGLLGLPRDMPAIRRG
LAYIRAEQAAEGPWFGRWGVNYLYGTGAVLPALAAIGEDMTQPYIAKACDWLVGCQQ
ENGGWGESCASYMEISSIGRGPTTPSQTAWALMGLVAANRRQDHDAIVRGCRYLIDLQ
QPDGRWDEKEFTGTGFPGYGVGQTIRLDDPALSKRLQQGAELSRAFMLRYDLYRQLFPI
MALSRAARVLKAAG
SEQ ID NO: 28 MflSHC1 NT Organism: Marinicaulis flavus
ATGTTCGATGAGAAAGAAGCAGGCGCTGATAAACGTTCGAATTTCGATCGTCAGTTA
ACGGCGTCGATTGAGGCAGCAGCAGACTGGCTCGCTCCCCGACAGAAACCGGAAGG
CTATTGGGTTGGTCGGCTGGAGTCCAACGCTTGCATGGAAGCACAGTGGATCTTGGC
GTTGTGGTTTATGGAATTAGAAGATCATCACCTGCGGCCGAGATTAGCAGCGTCGCT
GCGCGAAACCCAACGTCAGGATGGTTCGTGGGAAATCTACTACGGTGCTCCGGCCG
GCGATATTAACACTACGGTAGAAGCATACGCGGCGCTGCGTTCAATGGGCGATGAC
AAAGATGCGGCACACATGGTCCGTGCACGCGAATGGATCTTATCGAAAGGCGGCCT
GAAGAACATCAGAGTCTTTACCCGATACTGGCTTGCGTTAATAGGCGAATGGCCGTG
GAAGAAAACTCCGAATCTGCCGCCCGAAGTGATCTTCTTCCCGAATTGGTTCGTGTT
TTCTATCTATAACTTTGCGCAATGGGCCCGCGCCACCTTGATGCCGATTGCGGTTCTG
AGTGCACGTAGACCAAGCCGCCCGCTGCCAGCGGAGCGTCGCCTCGATGAACTTTTC
CCAGAAGGGCGTGAGAAATATAACTACGCCTACCCGGAGAAGCCTGGTGCCGGCTT
CGCCGAGGCGTTCTTCCTAACGGCGGACAAAATACTGCATGCTCTGCAAAGCTTCGG
TCAGACGGCGGGAATCGGTCTGCTGCGCAAAGGTGCCATCTCACGTGCGCTGGAAT
GGATTATCAAGCATCAGGATGCGGATGGGGTGTGGGGCGGCATCCAGCCACCATGG
ATCTATTCTCTGATGGCCCTGTATAACGAAGGGTATGCGCATGACCATAGTGTGGTG
GCGAAAGGTCTGGGCGCCCTTGATGATCCGCGGTGGCGTGTTGATCAGGGCGAAGC
CACGTGGATCCAGGCATCTACGAGCCCGGTGTGGGACACTGAACTGACCCTATTGG
CGCTGGAAGATTGTGACCTATCCGAGCGGCACGAGAAAGAGGTGGAAAAGGCCTTG
GATTGGTTGCTGAGCCAGCAGGTACTGCGCAAAGGGGACTGGAGTGTTAAATGCCC
GAAGCTGGAACCGGGCGGTTGGGCGTTCGAATATAAGAACTACTATTATCCTGACA
CTGATGACACCGCCGTTGCTCTGATCGCGCTCGCTCCCTTCCGTAATGATCCGAAAT
GGAAAGATAAAGGCATCGAAGAAGCCATTGAGAAAGGAGTGGAATGGCTCATCGG
TATGCAGTGCAAGAATGGTGGTTGGGGCGCGTTTGATAAAGACAACGACAAACAGA
TTCTGACCAAAATCCCATTCTGTGATTTTGGCGAGGCTCTGGATCCACCAAGCGTAG
ACGTCACCGCCCACATCATCGAAGCGTTTGGTAAGCTGGGCATTAGGAAAGACCAC
CCTGTAATGCTGCGCGCTCTGGACTATATCAAAACCGAGCAAGAGGCGGACGGGGC
GTGGTTCGGCCGGTGGGGCGTAAATTATGTGTATGGTACCGGTGCTGTGCTGCCCGC
GCTGGAAGCTATCGGGGAAGATATGACCCAGCATTATATTAAGAAGGCTTCGGACT
GGTTGATCCTGCACCAGAATGAGGATGGCGGCTGGGGTGAATCCTGCGCTTCATACA
TGGATGTCAAACAGATGGGTCGTGGTAAATCCACGGCTAGTCAAACGGCGTGGGCA
CTGATGGGTCTTGCGGCCGTCGGTCGTGCGGAAGATGAGCGTGCAATTGCGGACGG
TGTCCAATTTCTGATCGAGCGTCAGAAAGACGGAGCCTGGGAAGAGCCGGAATATA
CCGGGACCGGCTTCCCGGGATATGGCGTGGGTCAGCATATCAAATTGACCGATCCTC
AGTTGCAGGAGCGGCTTAAACAGGGTCCCGAACTGAGCCGCGCCTTTATGATCAACT
ATAACTTGTATCGACATTACTTTCCGATGATGGCAATGGGCCGCGTGCGGAAAATGA
TGGCAGGCGCT
SEQ ID NO: 29 MflSHC1 AA Organism: Marinicaulis flavus
MFDEKEAGADKRSNFDRQLTASIEAAADWLAPRQKPEGYWVGRLESNACMEAQWILA
LWFMELEDHHLRPRLAASLRETQRQDGSWEIYYGAPAGDINTTVEAYAALRSMGDDK
DAAHMVRAREWILSKGGLKNIRVFTRYWLALIGEWPWKKTPNLPPEVIFFPNWFVFSIY
NFAQWARATLMPIAVLSARRPSRPLPAERRLDELFPEGREKYNYAYPEKPGAGFAEAFF
LTADKILHALQSFGQTAGIGLLRKGAISRALEWIIKHQDADGVWGGIQPPWIYSLMALY
NEGYAHDHSVVAKGLGALDDPRWRVDQGEATWIQASTSPVWDTELTLLALEDCDLSE
RHEKEVEKALDWLLSQQVLRKGDWSVKCPKLEPGGWAFEYKNYYYPDTDDTAVALIA
LAPFRNDPKWKDKGIEEAIEKGVEWLIGMQCKNGGWGAFDKDNDKQILTKIPFCDFGE
ALDPPSVDVTAHIIEAFGKLGIRKDHPVMLRALDYIKTEQEADGAWFGRWGVNYVYGT
GAVLPALEAIGEDMTQHYIKKASDWLILHQNEDGGWGESCASYMDVKQMGRGKSTAS
QTAWALMGLAAVGRAEDERAIADGVQFLIERQKDGAWEEPEYTGTGFPGYGVGQHIK
LTDPQLQERLKQGPELSRAFMINYNLYRHYFPMMAMGRVRKMMAGA
SEQ ID NO: 30 AsySHC1 NT Organism: Acetobacter syzygii
ATGGGTAACTCACTTGCGCACACCGTTGCGGCGGCCTGTGACTGGCTGATCGGCGAG
CAAAAGGCTGATGGTCACTGGGTAGGCCCGGTGGCATCCAATGCAAGTATGGAAGC
GGAGTGGTGCTTGGCGCTTTGGTACCTGGGTCTGGAAGATCACCCTTTACGGCCTCG
CCTGGGGAACGCCCTGTTAGAAATGCAGCGCGAAGATGGGAGTTGGGGCACTTACT
GGGGCGCGGGCAATGGTGACATTAACGCCACCGTGGAAGCATACGCGGCTCTGCGT
TCCCTGGGGTACCCAGAGGATACACCGGCACTGTCCAAAGCGTGCGCGTGGATTAT
GCGCATGGGCGGCCTGCGCAATGTCCGGGTCTTTACGCGCTATTGGCTGGCCCTTAT
TGGTGAATGGCCGTGGGAACAGACTCCGAACCTACCGCCAGAAGTAATTTGGTTCCC
AAATAAGTTTGTTTTCTCGATTTACAATTTTGCACAATGGGCGCGCGCAACGCTGGT
TCCCCTGGCCATTCTGAGTGCACGTCGCCCATCGCGCCCGTTACGACCACAGGATCG
CCTGGATGCGTTGTTTCCAGGCGGGCGCGAAAATTTTGACTACGTTCTTCCGAAGAA
AGACGGGGTTGATCTGTGGAGTTCATTCTTCCGTACTACAGATCGTGGCTTACATTG
GCTCCAGACTCGGTTTCTGAAACGAAATACAGTCAGGGAAGCAGCAATACGTCACA
TGCTGGAATGGATTATCAGGCACCAGGATGCGGACGGCGGTTGGGGTGGCATTCAG
CCGCCGTGGGTGTACGGACTCATGGCCTTGCACGGCGAGGATTACCAGTTTCATCAC
CCCGTGATGGCAAAAGCGCTGGCAGCGCTGGATGACCCTGGCTGGCGCCAGGATCG
TGGTGACGCCTCATGGGTGCAAGCCACCAATTCACCAGTGTGGGATACCATGTTAGC
AATTATGGCGCTGCACGACGCCAAAGCCGAAGAACGGTATACCCCGCAGATGAATA
AAGCCCTTGATTGGCTGCTCGCTCGACAAGTACGCGTTAAAGGTGACTGGAGCATCA
AACTCCCGGAAGTTGAACCGGGTGGCTGGGCTTTCGAATATGCCAATGACCGTTACC
CTGACACCGACGACACGGCGGTGGCACTTATTGCGCTTTCGTCCTGTCGTCATCGGG
AAGAATGGAAACAGAAAGGAGTGGAAGCTGCCATTTTGCGCGGAGTGAACTGGCTT
ATTGCGATGCAAAGTACTTGCGGTGGTTGGGGCGCATTTGACAAAGATAACAATCG
CAGTCTTTTATCTAAAATCCCTTTCTGCGACTTTGGCGAAGCTCTTGACCCGCCGAGT
GTGGACGTCACCGCACATGTTCTTGAAGCGTTTGGGCTCCTGGGTCTTAGCCCGCAA
ACGCCGGCTATCCAGAGAGGGCTGGCGTACATTCGTGCTGAGCAGGAAGCATCGGG
CGCGTGGTTTGGACGCTGGGGTGTCAACTACTTGTACGGAACAGGAGCGGTCCTGCC
AGCGCTGGCTGCAATTGGAGAAGATATGACGCAGCCGTACATTACCCGTGCTTGCG
ATTGGTTGGTTGCCCACCAACAAGAGGACGGTGGTTGGGGTGAATCCTGTGCCTCTT
ATATGGACATTGCTAGTATCGGTCGAGGCACTACCACCCCGTCCCAAACGGCGTGG
GCGCTCATGGGTCTGATCGCGGTAAACCGCAAACAAGACCATGAAGCAATCGCGCG
CGGTTGTCGCTACTTAATAGACCGTCAGGAAAGCGATGGTAGCTGGACGGAGCAAG
AATTTACTGGTACTGGCTTTCCGGGATACGGTGTAGGCCAAACAATCAAGCTGGATG
ACCCGGCCGTTGCCAAGCGTCTGCAACAGGGCGCGGAACTTTCTCGGGCGTTTATGC
TGCGCTATGATCTGTACCGACAGTTCTTTCCGCTCATGGCTCTGAGTAGAGCAGCTA
GGATTATTAGTTTTGAGCAG
SEQ ID NO: 31 AsySHC1 AA Organism: Acetobacter syzygii
MGNSLAHTVAAACDWLIGEQKADGHWVGPVASNASMEAEWCLALWYLGLEDHPLRP
RLGNALLEMQREDGSWGTYWGAGNGDINATVEAYAALRSLGYPEDTPALSKACAWIM
RMGGLRNVRVFTRYWLALIGEWPWEQTPNLPPEVIWFPNKFVFSIYNFAQWARATLVP
LAILSARRPSRPLRPQDRLDALFPGGRENFDYVLPKKDGVDLWSSFFRTTDRGLHWLQT
RFLKRNTVREAAIRHMLEWIIRHQDADGGWGGIQPPWVYGLMALHGEDYQFHHPVMA
KALAALDDPGWRQDRGDASWVQATNSPVWDTMLAIMALHDAKAEERYTPQMNKAL
DWLLARQVRVKGDWSIKLPEVEPGGWAFEYANDRYPDTDDTAVALIALSSCRHREEW
KQKGVEAAILRGVNWLIAMQSTCGGWGAFDKDNNRSLLSKIPFCDFGEALDPPSVDVT
AHVLEAFGLLGLSPQTPAIQRGLAYIRAEQEASGAWFGRWGVNYLYGTGAVLPALAAI
GEDMTQPYITRACDWLVAHQQEDGGWGESCASYMDIASIGRGTTTPSQTAWALMGLIA
VNRKQDHEAIARGCRYLIDRQESDGSWTEQEFTGTGFPGYGVGQTIKLDDPAVAKRLQ
QGAELSRAFMLRYDLYRQFFPLMALSRAARIISFEQ
SEQ ID NO: 32 BstSHC1 NT Organism: Bradyrhizobium sp. STM 3843
ATGACACTGAATGGCAAGACATTAGCGGCGACTGTCTCGGCACCTGACAACTTTCAG
ACGGCCCTGCACTCGACGGTTCGCGCCGCTGCCGACTGGCTGATTGCTCATCAGAAA
GGTGATGGCCACTGGGTAGGACGTGCGGAATCGAACGCCTGTATGGAAGCACAGTG
GTGCTTAGCACTGTGGTTCATGGGGCTGGAAGATCATCCGCTGCGTCCTAGAATGGG
CCAAGCGCTTCTGCAAACACAGCGTGCGGACGGCAGCTGGCAGATTTACTTCAACG
CGCCGAATGGTGATATAAACGCAACGGTCGAGGCGTACGCCGCATTACGCTCGTTA
GGGCACCGTGATGATGAACCTGTACTGCAAAGAGCACGCCAGTGGATTGAGGCCAA
AGGCGGACTGCGCAACGTCCGCGTGTTTACCCGTTATTGGCTCGCGCTGCTGGGTGA
GTGGCCGTGGGAAAAGGTCCCTAATATTCCGCCAGAGGTTATCTGGTTTCCGGTGTG
GTTTCCGTTCAGCATCTACAATTTTGCCCAGTGGGCGAGGGCAACTCTAATGCCAAT
TGCAATTTTGAGTGCCCGCCGTCCAAGCCGTCCGCTGCCGGCGGAGAACCGCCTGGA
TGCCCTCTTCCCGAAGGGTCGCGGGGCTTTTGATTATGAACTCCCGGTGAAAGCTGA
TGCAGGCGGTTGGGATAAATTCTTCCGCGGTGCCGACAAAGTTCTGCATGCGCTGCA
AAACTTGGGCAATTGGTTGAATCTGGGCTTGTGGCGTCCGGCTGCTATGTCGCGTGT
CCTGGAATGGATGATTCGCCACCAGGATTTCGACGGTGCCTGGGGCGGTATTCAGCC
ACCGTGGATTTATGGCTTGATGGCACTGTACGTCGAAGGATACCCTATCAACCACCC
CGTTGTTGCCAAAGGCCTCGATGCGCTAAACGACCCGGGATGGCGTGTGGACATCG
GTGAGGCGACTTATATTCAGGCGACGAACTCTCCTGTGTGGGATACCATCCTAACCT
TACTCGCTTTTGATGATGCGGGTGTGATTGGAGATTATCCAGAGGCGGTCGAGAAAG
CCGTGAACTGGGTTCTGGCACGACAAGTTCGTGTCCCGGGCGATTGGAGTGTTAAAC
TGCCTCATGTGAAACCCGGCGGGTGGGCTTTCGAGTACGCGAACAACTATTATCCAG
ACACTGACGACACCGCGGTCGCCTTGATTGCGCTGGCCCCATTACGCCATGACCCGA
AATGGCAGGCGAAAGGCATTGATGAAGCAATCCAACTGGGCGTCGATTGGTTAATT
GGTATGCAGAGCCAGTCAGGTGGCTGGGGCGCCTTTGATAAAGACAACAACCAACA
GATACTGACGAAAATTCCCTTCTGCGACTTTGGCGAAGCTCTGGATCCGCCATCCGT
GGATGTGACAGCCCATGTGATAGAGGCGTTTGGCAAACTTGGACTGTCAAGGAACC
ATCCTACCATGGTGCGGGCGCTTGATTACGTGCGCCGTGAGCAGGAACCATCCGGCC
CCTGGTTCGGTCGCTGGGGCGTAAACTATGTGTATGGTACGGGAGCAGTCCTTCCGG
CGCTGGCGGCTATCGGTGAGGACATGAGTCAGCCTTACATCCAGCGCGCATGCGAG
TGGTTAGTGGCGCAACAGCAAGATAACGGTGGGTGGGGTGAATCATGTGCGTCTTA
TATGGACATTTCTGCTGTGGGCCGTGGGACTGCCACCGCGAGTCAAACCGCCTGGGC
CCTGATGGCGCTTTTGGCTGCCAACCGTCCGGGTGACCGAGAGGCGATCGAAAGAG
GGTGCCTCTGGCTGATAGAACATCAGAGCGCTGGTACCTGGGCGGAACCAGAGTTC
ACTGGCACGGGGTTCCCAGGCTATGGCGTCGGCCAAACTATCAAATTAACAGATCC
AAGTCTCCAGCAACGCCTGATGCAGGGGCCAGAACTGTCTCGTGCTTTTATGCTGCG
TTATGGTATGTATTGCCACTACTTTCCGCTGATGGCGTTAGGTCGCACACTGCGGCCT
CAGGCGCACGGCAAAGCGCAGTCCACTCCAGCGGCGAACGCCGTTATT
SEQ ID NO: 33 BstSHC1 AA Organism: Bradyrhizobium sp. STM 3843
MTLNGKTLAATVSAPDNFQTALHSTVRAAADWLIAHQKGDGHWVGRAESNACMEAQ
WCLALWFMGLEDHPLRPRMGQALLQTQRADGSWQIYFNAPNGDINATVEAYAALRSL
GHRDDEPVLQRARQWIEAKGGLRNVRVFTRYWLALLGEWPWEKVPNIPPEVIWFPVWF
PFSIYNFAQWARATLMPIAILSARRPSRPLPAENRLDALFPKGRGAFDYELPVKADAGG
WDKFFRGADKVLHALQNLGNWLNLGLWRPAAMSRVLEWMIRHQDFDGAWGGIQPP
WIYGLMALYVEGYPINHPVVAKGLDALNDPGWRVDIGEATYIQATNSPVWDTILTLLAF
DDAGVIGDYPEAVEKAVNWVLARQVRVPGDWSVKLPHVKPGGWAFEYANNYYPDTD
DTAVALIALAPLRHDPKWQAKGIDEAIQLGVDWLIGMQSQSGGWGAFDKDNNQQILTK
IPFCDFGEALDPPSVDVTAHVIEAFGKLGLSRNHPTMVRALDYVRREQEPSGPWFGRWG
VNYVYGTGAVLPALAAIGEDMSQPYIQRACEWLVAQQQDNGGWGESCASYMDISAVG
RGTATASQTAWALMALLAANRPGDREAIERGCLWLIEHQSAGTWAEPEFTGTGFPGYG
VGQTIKLTDPSLQQRLMQGPELSRAFMLRYGMYCHYFPLMALGRTLRPQAHGKAQSTP
AANAVI
SEQ ID NO: 34 GfrSHC2 NT Organism: Gluconobacter frateurii NBRC 101659
ATGAGCGCACCGATTCTCAAAGGCATGAGCAACAGCTTAGCTCATACGGTGAGTTGT
GCGTGTGATTGGCTCATTGGTCAACAGAAGGGTGATGGCCACTGGGTCGGTAGCGT
GGAGAGCAATGCTTCTATGGAAGCCGAATGGTGCCTGGCACTCTGGTTCTTGGGTCT
GGAAGATCATCCGTTGCGTCCGCGTTTGGGGAATGCGCTGCTGGAAATGCAGCGTG
ATGACGGCTCCTGGGGCGTGTACCTGGGCGCGCGTAGCGGCGACATCAATGCTACC
GTAGAGGCCTATGCGGCGTTACGTTCACTCGGGTACTCCGCAAACTCTCCAGTCCTG
CTGAAGGCTTCGATGTGGATTGCTGAAAAGGGCGGATTAAAGAATATTCGGGTATTC
ACCCGCTACTGGTTAGCCCTGATCGGCGAATGGCCTTGGGAGAAAACACCAAATTTG
CCGCCGGAAGTAATCTGGTTCCCGAATAACTTTGTCTTTAGCATCTATAATTTTGCGC
AATGGGCGCGAGCGACTCTGGCCCCGTTAGCCATCTTATCCGCCCGTCGTCCAAGCC
GTCCATTGCGCCCTCAGGATAGACTGGATGCGCTGTTCCCAGAAGGCCGCGAGAAA
TTCGATTACACGCTTCCGAAGAAGGATCGCGTTGACTTATGGTCGTCCTTCTTTCGAA
CCACCGATAAAGGTTTGCACTGGCTGCAAAGCCGCTTTCTGAAACGTAACACCGTTC
GCGAAGCTGCCATTCGCCACATGCTCGAATGGATTATTCGTCACCAAGACGCAGATG
GCGGGTGGGGCGGTATCCAGCCGCCGTGGGTCTACGGCCTCATGGCGTTGCATGGA
GAAGGCTATCCTTTCCATCATCCTGTTCTGGCCAAAGCGTTAGCTGCCCTGGACGAC
CCGGGTTGGCGCTATGATCGCGGCGAAGCATCATGGATTCAGGCCACCAACTCCCC
GGTGTGGGATACGATGCTGGCACTTATGGCACTGTACGATGCAAACGCACAAGAAC
GTTTTACGCCGGAAATGGACAAAGCTCTGGAGTGGCTGCTCAACCGCCAGGTGCGC
GTGAAAGGCGACTGGTCCATAAAACTCCCCGATGTTGAGCCGGGCGGTTGGAGCTTT
GAATATGCGAATGATCGTTATCCAGATACCGATGATACTGCTGTTGCGTTGATCGCA
CTGTCTTTCTGTCGCCACAAAGAAGAATGGAAACGCAAAGGCGTCGATGAAGCGAT
TGATCGCGCAGTCAACTGGCTGATCGCAATGCAATCATCGTGCGGTGGTTGGGGCGC
GTTCGACAAAGATAACAACAAATCCCTGCTTAGCAAGATCCCGTTCTGCGACTTCGG
TGAGGCTTTGGATCCGCCATCGGTTGATGTCACGGCCCATATCTTAGAGGCATTTGG
TTTGCTTGGTCTGTCGCGCGACTTGCCCGCGGTCCAGAAAGCCTTAGCCTACGTTCGT
TCTGAACAAGATCCGCAGGGGCCTTGGTTTGGACGTTGGGGCGTCAACTATCTGTAT
GGTACAGGCGCGGTCCTGCCGGCGCTGGCCGCCATTGGCGAAGATATGACTCAACC
CTACATTACGAACGCGTGCGACTGGTTAATATCTTGTCAGCAGGATGATGGCGGCTG
GGGTGAAAGTTGCGCCAGTTATATGGACATTTCGTCCATTGGGCGCGGCTCGACGAC
TGCCAGCCAGACAGCGTGGGCGCTTATGGGCCTTATCGCTGTGGGTCGCCCGCAGG
ATTACGAAGCCATTGCGAAGGGCTGCCGCTTTCTGATCGATCGGCAAGAGGCGGAC
GGCAGTTGGACCGAGCAGGAGTTTACGGGCACAGGTTTTCCTGGTTATGGCGTAGG
ACAGACCATTAAGCTTGATGATCCGGCCTTGAGTAAACGTTTGATGCAGGGTGCGGA
ACTGAGCCGCGCTTTTATGTTGCGTTACGACATGTATCGTCAGTACTTTCCGATTATG
GCGCTGGCCCGCGCGAATCGCTTGCTCTCCCAGGATGTT
SEQ ID NO: 35 GfrSHC2 AA Organism: Gluconobacter frateurii NBRC 101659
MSAPILKGMSNSLAHTVSCACDWLIGQQKGDGHWVGSVESNASMEAEWCLALWFLGL
EDHPLRPRLGNALLEMQRDDGSWGVYLGARSGDINATVEAYAALRSLGYSANSPVLLK
ASMWIAEKGGLKNIRVFTRYWLALIGEWPWEKTPNLPPEVIWFPNNFVFSIYNFAQWAR
ATLAPLAILSARRPSRPLRPQDRLDALFPEGREKFDYTLPKKDRVDLWSSFFRTTDKGLH
WLQSRFLKRNTVREAAIRHMLEWIIRHQDADGGWGGIQPPWVYGLMALHGEGYPFHH
PVLAKALAALDDPGWRYDRGEASWIQATNSPVWDTMLALMALYDANAQERFTPEMD
KALEWLLNRQVRVKGDWSIKLPDVEPGGWSFEYANDRYPDTDDTAVALIALSFCRHKE
EWKRKGVDEAIDRAVNWLIAMQSSCGGWGAFDKDNNKSLLSKIPFCDFGEALDPPSVD
VTAHILEAFGLLGLSRDLPAVQKALAYVRSEQDPQGPWFGRWGVNYLYGTGAVLPALA
AIGEDMTQPYITNACDWLISCQQDDGGWGESCASYMDISSIGRGSTTASQTAWALMGLI
AVGRPQDYEAIAKGCRFLIDRQEADGSWTEQEFTGTGFPGYGVGQTIKLDDPALSKRLM
QGAELSRAFMLRYDMYRQYFPIMALARANRLLSQDV
SEQ ID NO: 36 AghSHC1:CP3 NT Organism: Acetobacter ghanensis
ATGGCAACCCTTCGCGAAGCCGCGATTAAACACATGCTGGAATGGATTATTCGCCATCAG
GACGCAGACGGCGGTTGGGGCGGCATTCAGCCGCCATGGGTGTACGGCTTAATGGCACT
ACATGGTGAAGATTATCAGTTCCACCATCCGGTGATGGCTAAGGCTTTAGCCGCCTTAGAC
GATCCTGGCTGGCGTCAAGATCGTGGGGATGCCAGCTGGGTGCAGGCCACGAACAGTCC
AGTATGGGATACGATGTTAGCACTGATGGCGCTCCACGACGCGGGCGCTGAAGAGCGCT
ATACCCCTGAAATGGATAAAGCGCTGGATTGGCTGTTACAGCGCCAGGTTCGGGTAACAG
GTGACTGGTCAATCAAACTGCCGGGAGTCGAACCTGGCGGTTGGGCATTCGAATATGCGA
ATGACCGTTATCCCGATACCGACGATACCGCGGTTGCGCTCATTGCACTGAGCGCGTGTC
GCCACCGGGAAGAATGGAAGAAGAAAGGGGTCGAAGCTGCGATCACACGCGGAGTAAAT
TGGCTCATTGCCATGCAGTCAACGTGCGGCGGCTGGGGCGCATTTGATAAAGATAACAAT
CGTTCGTTACTGTCGAAGATTCCATTTTGTGACTTTGGCGAAGCTCTGGACCCACCCAGCG
TCGATGTCACGGCACATGTGCTTGAAGCCTTTGGTCTGCTGGGTCTGCCCCGCGAAACCC
CTTCCATTCAGCGCGGCCTTGCTTACATCAGAGCGGAACAGGAGCCGTCTGGAGCTTGGT
TTGGCCGATGGGGCGTTAATTACCTGTATGGGACCGGGGCAGTCCTACCGGCCCTGGCG
GCAATCGGTGAGGATATGACCCAGCCGTACATCACTCGTGCGTGTGATTGGCTCGTCGCT
CATCAGCAGGAAAACGGTGGCTGGGGTGAATCGTGTGCGTCATATATGGACGTCGCGTC
GATTGGTCATGGAACTCCGACCGCCTCTCAGACCGCATGGGCCCTGATGGGACTGATTGC
GGTGAATCGACCGCAGGATCACGAGGCAATCGCACGCGGCTGTCGTTTTCTGATTGACCG
TCAGGAAGAAGATGGAAGTTGGACCGAAGAGGAATTTACCGGTACCGGCTTTCCAGGCTA
CGGTGTCGGCCAGACTATCAAACTGGACGATCCAGCCGTCGCTAAACGTCTGCAACAGG
GTGCGGAACTTTCGCGTGCGTTCATGCTACGCTACGACCTGTATCGTCAGTTCTTCCCGCT
GATGGCCCTGAGCCGTGCGGCGCGTATCATTGGTGGCGGTAGCGGTGGCGGTAGCTCG
CTTGCGCACACGGTCGCAGCGGCGTGTGATTGGCTGATCGGGGAGCAGAAAGCCGATGG
ACATTGGGTGGGTCCGGTAGCGTCCAATGCATCAATGGAAGCGGAATGGTGCCTGGCTTT
ATGGTACCTGGGTCTGGAAGATCACCCGCTGCGCCCACGTTTAGGCAACGCCCTGTTGCA
GATGCAGCGAGAGGACGGTTCTTGGGGCATTTACTGGGGCGCCGGGAACGGTGACATTA
ATGCAACGGTAGAAGCTTACGCGGCACTACGTTCGTTAGGCTACCCGGCTGACACGCCAG
CTTTAAGCAAAGCGTGTGCGTGGATTATGCGTATGGGCGGCCTGCGCAATATCCGCGTGT
TCACTCGTTACTGGCTCGCGCTGATTGGCGAATGGCCGTGGGAACAGACGCCGAACCTG
CCGCCGGAGATCATCTGGTTTCCGAATAAATTCGTTTTCAGCATTTACAACTTTGCACAGTG
GGCCCGTGCAACATTAGTGCCTCTGGCAATTCTGAGTGCAAGACGCCCGTCACGTCCTCT
CCGCCCGCAGGATCGTCTGGATGCGTTGTTCCCGGGCGGTCGGGAAAATTTCGACTATGT
GCTGCCTAAACGCGATGGCATGGACCTGTGGTCCTCATTCTTTCGCACCACGGACCGCGG
CCTGCACTGGCTGCAATCAAAATTTCTGGGCGCGTAA
SEQ ID NO: 37 AghSHC1:CP3 AA Organism: Acetobacter ghanensis
MATLREAAIKHMLEWIIRHQDADGGWGGIQPPWVYGLMALHGEDYQFHHPVMAKALAALD
DPGWRQDRGDASWVQATNSPVWDTMLALMALHDAGAEERYTPEMDKALDWLLQRQVRVT
GDWSIKLPGVEPGGWAFEYANDRYPDTDDTAVALIALSACRHREEWKKKGVEAAITRGVNWL
IAMQSTCGGWGAFDKDNNRSLLSKIPFCDFGEALDPPSVDVTAHVLEAFGLLGLPRETPSIQR
GLAYIRAEQEPSGAWFGRWGVNYLYGTGAVLPALAAIGEDMTQPYITRACDWLVAHQQENG
GWGESCASYMDVASIGHGTPTASQTAWALMGLIAVNRPQDHEAIARGCRFLIDRQEEDGSWT
EEEFTGTGFPGYGVGQTIKLDDPAVAKRLQQGAELSRAFMLRYDLYRQFFPLMALSRAARIIG
GGSGGGSSLAHTVAAACDWLIGEQKADGHWVGPVASNASMEAEWCLALWYLGLEDHPLRP
RLGNALLQMQREDGSWGIYWGAGNGDINATVEAYAALRSLGYPADTPALSKACAWIMRMGG
LRNIRVFTRYWLALIGEWPWEQTPNLPPEIIWFPNKFVFSIYNFAQWARATLVPLAILSARRPSR
PLRPQDRLDALFPGGRENFDYVLPKRDGMDLWSSFFRTTDRGLHWLQSKFLGA
SEQ ID NO: 38 AghSHC1:CP6 NT Organism: Acetobacter ghanensis
ATGGCAGAATGGAAGAAGAAAGGGGTCGAAGCTGCGATCACACGCGGAGTAAATTGGCT
CATTGCCATGCAGTCAACGTGCGGCGGCTGGGGCGCATTTGATAAAGATAACAATCGTTC
GTTACTGTCGAAGATTCCATTTTGTGACTTTGGCGAAGCTCTGGACCCACCCAGCGTCGAT
GTCACGGCACATGTGCTTGAAGCCTTTGGTCTGCTGGGTCTGCCCCGCGAAACCCCTTCC
ATTCAGCGCGGCCTTGCTTACATCAGAGCGGAACAGGAGCCGTCTGGAGCTTGGTTTGGC
CGATGGGGCGTTAATTACCTGTATGGGACCGGGGCAGTCCTACCGGCCCTGGCGGCAAT
CGGTGAGGATATGACCCAGCCGTACATCACTCGTGCGTGTGATTGGCTCGTCGCTCATCA
GCAGGAAAACGGTGGCTGGGGTGAATCGTGTGCGTCATATATGGACGTCGCGTCGATTG
GTCATGGAACTCCGACCGCCTCTCAGACCGCATGGGCCCTGATGGGACTGATTGCGGTG
AATCGACCGCAGGATCACGAGGCAATCGCACGCGGCTGTCGTTTTCTGATTGACCGTCAG
GAAGAAGATGGAAGTTGGACCGAAGAGGAATTTACCGGTACCGGCTTTCCAGGCTACGGT
GTCGGCCAGACTATCAAACTGGACGATCCAGCCGTCGCTAAACGTCTGCAACAGGGTGC
GGAACTTTCGCGTGCGTTCATGCTACGCTACGACCTGTATCGTCAGTTCTTCCCGCTGATG
GCCCTGAGCCGTGCGGCGCGTATCATTGGTGGCGGTAGCGGTGGCGGTAGCTCGCTTG
CGCACACGGTCGCAGCGGCGTGTGATTGGCTGATCGGGGAGCAGAAAGCCGATGGACAT
TGGGTGGGTCCGGTAGCGTCCAATGCATCAATGGAAGCGGAATGGTGCCTGGCTTTATG
GTACCTGGGTCTGGAAGATCACCCGCTGCGCCCACGTTTAGGCAACGCCCTGTTGCAGAT
GCAGCGAGAGGACGGTTCTTGGGGCATTTACTGGGGCGCCGGGAACGGTGACATTAATG
CAACGGTAGAAGCTTACGCGGCACTACGTTCGTTAGGCTACCCGGCTGACACGCCAGCTT
TAAGCAAAGCGTGTGCGTGGATTATGCGTATGGGCGGCCTGCGCAATATCCGCGTGTTCA
CTCGTTACTGGCTCGCGCTGATTGGCGAATGGCCGTGGGAACAGACGCCGAACCTGCCG
CCGGAGATCATCTGGTTTCCGAATAAATTCGTTTTCAGCATTTACAACTTTGCACAGTGGG
CCCGTGCAACATTAGTGCCTCTGGCAATTCTGAGTGCAAGACGCCCGTCACGTCCTCTCC
GCCCGCAGGATCGTCTGGATGCGTTGTTCCCGGGCGGTCGGGAAAATTTCGACTATGTG
CTGCCTAAACGCGATGGCATGGACCTGTGGTCCTCATTCTTTCGCACCACGGACCGCGGC
CTGCACTGGCTGCAATCAAAATTTCTGAAACGCAACACCCTTCGCGAAGCCGCGATTAAAC
ACATGCTGGAATGGATTATTCGCCATCAGGACGCAGACGGCGGTTGGGGCGGCATTCAG
CCGCCATGGGTGTACGGCTTAATGGCACTACATGGTGAAGATTATCAGTTCCACCATCCG
GTGATGGCTAAGGCTTTAGCCGCCTTAGACGATCCTGGCTGGCGTCAAGATCGTGGGGAT
GCCAGCTGGGTGCAGGCCACGAACAGTCCAGTATGGGATACGATGTTAGCACTGATGGC
GCTCCACGACGCGGGCGCTGAAGAGCGCTATACCCCTGAAATGGATAAAGCGCTGGATT
GGCTGTTACAGCGCCAGGTTCGGGTAACAGGTGACTGGTCAATCAAACTGCCGGGAGTC
GAACCTGGCGGTTGGGCATTCGAATATGCGAATGACCGTTATCCCGATACCGACGATACC
GCGGTTGCGCTCATTGCACTGAGCGCGTGTCGCCACGGCGCGTAA
SEQ ID NO: 39 AghSHC1:CP6 AA Organism: Acetobacter ghanensis
MAEWKKKGVEAAITRGVNWLIAMQSTCGGWGAFDKDNNRSLLSKIPFCDFGEALDPPS
VDVTAHVLEAFGLLGLPRETPSIQRGLAYIRAEQEPSGAWFGRWGVNYLYGTGAVLPA
LAAIGEDMTQPYITRACDWLVAHQQENGGWGESCASYMDVASIGHGTPTASQTAWAL
MGLIAVNRPQDHEAIARGCRFLIDRQEEDGSWTEEEFTGTGFPGYGVGQTIKLDDPAVA
KRLQQGAELSRAFMLRYDLYRQFFPLMALSRAARIIGGGSGGGSSLAHTVAAACDWLI
GEQKADGHWVGPVASNASMEAEWCLALWYLGLEDHPLRPRLGNALLQMQREDGSWG
IYWGAGNGDINATVEAYAALRSLGYPADTPALSKACAWIMRMGGLRNIRVFTRYWLA
LIGEWPWEQTPNLPPEIIWFPNKFVFSIYNFAQWARATLVPLAILSARRPSRPLRPQDRLD
ALFPGGRENFDYVLPKRDGMDLWSSFFRTTDRGLHWLQSKFLKRNTLREAAIKHMLEW
IIRHQDADGGWGGIQPPWVYGLMALHGEDYQFHHPVMAKALAALDDPGWRQDRGDA
SWVQATNSPVWDTMLALMALHDAGAEERYTPEMDKALDWLLQRQVRVTGDWSIKLP
GVEPGGWAFEYANDRYPDTDDTAVALIALSACRHGA

As is evident from the foregoing description, certain aspects of the present disclosure are not limited by the particular details of the examples provided herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

I/We claim:

1. A process of producing ambroxide or an ambroxide derivative from homofarnesol, comprising use of:
a. a polypeptide with the sequence of SEQ ID NOs.: 3, 5, 7, 9, 11, 13, 15, 17, 19, 21, 23, 25, 27, 29, 31, 33, 35, 37, or 39;
b. a polypeptide with a sequence that is at least 80%, 85%, 90%, 91%, 92%, 93%, 94%, 95%, 96%, 97%, 98%, 99% or 100% identical to the sequence of SEQ ID NOs.: 3, 37, or 39; or
c. a chimeric polypeptide, such as a circular permutant, comprising a functional fragment or domain of a polypeptide with the sequence of SEQ ID NOs.: 3, 5, 7, 9, 11, 13, 15, 17, 19, 21, 23, 25, 27, 29, 31, 33, or 35.

2. The process of claim 1, wherein the polypeptide is expressed from a nucleic acid molecule with a sequence that is at least 90%, 91%, 92%, 93%, 94%, 95%, 96%, 97%, 98%, 99% or 100% identical to the sequence of SEQ ID NO.: 2, 36, or 38.

3. The process of claim 1, wherein the polypeptide is isolated from or produced in a/an Escherichia coli, Acetobacter ghanensis, Alicyclobacillus tengchongensis, Acetobacter pasteurianus, Phaeospirillum fulvum, Komagataeibacter diospyri, Acetobacter orleanensis, Komagataeibacter rhaeticus, Acetobacter malorum, Rhodoblastus acidophilus, Amphiplicatus metriothermophilus, Acetobacter sp. DsW_54, Rhodopseudomonas faecalis, Komagataeibacter medellinensis, Marinicaulis flavus, Acetobacter syzygii, Bradyrhizobium sp. STM 3843, or Gluconobacter frateurii NBRC 101659 microorganism.

4. The process of claim 1, wherein the polypeptide is expressed by in vitro translation or by expressing the polypeptide in a host microorganism or cell.

5. The process of claim 4, wherein the host cell is a bacterium or yeast cell.

6. The process of claim 4, wherein the host cell is Escherichia coli, Saccharomyces cerevisiae, or Yarrowia lipolytica.

7. The process of claim 1, wherein catalysis of homofarnesol to ambroxide takes place in a growth media, buffer (e.g., bioconversion buffer) or solution (e.g., bioconversion solution), and the process comprises contacting homofarnesol with the polypeptide or host microorganism or cell.

8. The process of claim 1, wherein the process comprises adding homofarnesol to host microorganisms or cells expressing or containing the polypeptide.

9. The process of claim 1, wherein the process comprises adding homofarnesol to cell lysate.

10. The process of claim 1, wherein the process comprises adding homofarnesol to isolated polypeptide in vitro.

11. The process of claim 1, wherein one or more or all of the steps of the process is performed at a temperature of 16-42° C., a pH of 5.4-8, a substrate concentration of 1-500 g/L, and/or a SDS concentration of 0.1-5%.

12. The process of claim 1, wherein the yield of ambroxide is within 1-500 g/L.

13. The process of claim 1, wherein the process further comprises harvesting the ambroxide, such as from the growth media, buffer or solution.

14. The process according to claim 13, wherein the ambroxide is harvested using organic solvents, steam extraction/distillation or filtration.

15. The process according to claim 14, wherein the organic solvent is hexane, ethyl acetate, ethanol, or methyl tert-butyl ether.

16.-19. (canceled)

20. An isolated recombinant host microorganism or cell that comprises a polynucleotide with a sequence that encodes any one of the polypeptides as defined in claim 1 or the polynucleotide as defined herein.

21.-27. (canceled)

28. A composition comprising any one of the polypeptides defined herein, such as in claim 1.

29. (canceled)

30. A composition comprising any one of the polynucleotides provided herein, such as defined in claim 2.

31.-32. (canceled)

33. A vector comprising any one of the polynucleotides provided herein, such as defined in claim 20.

34.-35. (canceled)

36. A composition comprising ambroxide produced by any one of the processes provided herein, such as the process of claim 1 or any one of the host microorganisms or cells provided herein.


SEQUENCE LISTING (53 sequences)

SEQ ID NO: 1 - DNA, 6227 bp, synthetic construct
  source 1..6227: mol_type = other DNA
gctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggtatgggagaccgaaagtgaaacgtgatttcatgcgtcattttgaacattttgtaaatcttatttaataatgtgtgcggcaattcacatttaatttatgaatgttttcttaacatcgcggcaactcaagaaacggcaggttcggatcttagctactagagaaagaggagaaatactagatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgaccaggcatcaaataaaacgaaaggctcagtcgaaagactgggcctttcgttttatctgttgtttgtcggtgaacgctctctactagagtcacactggctcaccttcgggtgggcctttctgcgtttataggtctctagcg

SEQ ID NO: 2 - DNA, 2091 bp, Acetobacter ghanensis
  source 1..2091: mol_type = genomic DNA
atgaatacgatctctcatccgagtaaagtgaaagcagccgttagtgatagccacactcccacctcagcgacgccgaccccattcaagggtatggacaactcgcttgcgcacacggtcgcagcggcgtgtgattggctgatcggggagcagaaagccgatggacattgggtgggtccggtagcgtccaatgcatcaatggaagcggaatggtgcctggctttatggtacctgggtctggaagatcacccgctgcgcccacgtttaggcaacgccctgttgcagatgcagcgagaggacggttcttggggcatttactggggcgccgggaacggtgacattaatgcaacggtagaagcttacgcggcactacgttcgttaggctacccggctgacacgccagctttaagcaaagcgtgtgcgtggattatgcgtatgggcggcctgcgcaatatccgcgtgttcactcgttactggctcgcgctgattggcgaatggccgtgggaacagacgccgaacctgccgccggagatcatctggtttccgaataaattcgttttcagcatttacaactttgcacagtgggcccgtgcaacattagtgcctctggcaattctgagtgcaagacgcccgtcacgtcctctccgcccgcaggatcgtctggatgcgttgttcccgggcggtcgggaaaatttcgactatgtgctgcctaaacgcgatggcatggacctgtggtcctcattctttcgcaccacggaccgcggcctgcactggctgcaatcaaaatttctgaaacgcaacacccttcgcgaagccgcgattaaacacatgctggaatggattattcgccatcaggacgcagacggcggttggggcggcattcagccgccatgggtgtacggcttaatggcactacatggtgaagattatcagttccaccatccggtgatggctaaggctttagccgccttagacgatcctggctggcgtcaagatcgtggggatgccagctgggtgcaggccacgaacagtccagtatgggatacgatgttagcactgatggcgctccacgacgcgggcgctgaagagcgctatacccctgaaatggataaagcgctggattggctgttacagcgccaggttcgggtaacaggtgactggtcaatcaaactgccgggagtcgaacctggcggttgggcattcgaatatgcgaatgaccgttatcccgataccgacgataccgcggttgcgctcattgcactgagcgcgtgtcgccaccgggaagaatggaagaagaaaggggtcgaagctgcgatcacacgcggagtaaattggctcattgccatgcagtcaacgtgcggcggctggggcgcatttgataaagataacaatcgttcgttactgtcgaagattccattttgtgactttggcgaagctctggacccacccagcgtcgatgtcacggcacatgtgcttgaagcctttggtctgctgggtctgccccgcgaaaccccttccattcagcgcggccttgcttacatcagagcggaacaggagccgtctggagcttggtttggccgatggggcgttaattacctgtatgggaccggggcagtcctaccggccctggcggcaatcggtgaggatatgacccagccgtacatcactcgtgcgtgtgattggctcgtcgctcatcagcaggaaaacggtggctggggtgaatcgtgtgcgtcatatatggacgtcgcgtcgattggtcatggaactccgaccgcctctcagaccgcatgggccctgatgggactgattgcggtgaatcgaccgcaggatcacgaggcaatcgcacgcggctgtcgttttctgattgaccgtcaggaagaagatggaagttggaccgaagaggaatttaccggtaccggctttccaggctacggtgtcggccagactatcaaactggacgatccagccgtcgctaaacgtctgcaacagggtgcggaactttcgcgtgcgttcatgctacgctacgacctgtatcgtcagttcttcccgctgatggccctgagccgtgcggcgcgtatcattccaattaagaac

SEQ ID NO: 3 - protein, 697 aa, Acetobacter ghanensis
MNTISHPSKVKAAVSDSHTPTSATPTPFKGMDNSLAHTVAAACDWLIGEQKADGHWVGPVASNASMEAEWCLALWYLGLEDHPLRPRLGNALLQMQREDGSWGIYWGAGNGDINATVEAYAALRSLGYPADTPALSKACAWIMRMGGLRNIRVFTRYWLALIGEWPWEQTPNLPPEIIWFPNKFVFSIYNFAQWARATLVPLAILSARRPSRPLRPQDRLDALFPGGRENFDYVLPKRDGMDLWSSFFRTTDRGLHWLQSKFLKRNTLREAAIKHMLEWIIRHQDADGGWGGIQPPWVYGLMALHGEDYQFHHPVMAKALAALDDPGWRQDRGDASWVQATNSPVWDTMLALMALHDAGAEERYTPEMDKALDWLLQRQVRVTGDWSIKLPGVEPGGWAFEYANDRYPDTDDTAVALIALSACRHREEWKKKGVEAAITRGVNWLIAMQSTCGGWGAFDKDNNRSLLSKIPFCDFGEALDPPSVDVTAHVLEAFGLLGLPRETPSIQRGLAYIRAEQEPSGAWFGRWGVNYLYGTGAVLPALAAIGEDMTQPYITRACDWLVAHQQENGGWGESCASYMDVASIGHGTPTASQTAWALMGLIAVNRPQDHEAIARGCRFLIDRQEEDGSWTEEEFTGTGFPGYGVGQTIKLDDPAVAKRLQQGAELSRAFMLRYDLYRQFFPLMALSRAARIIPIKN

SEQ ID NO: 4 - DNA, 1899 bp, unidentified
  source 1..1899: mol_type = genomic DNA; note = Alicyclobacillus tengchongensis
atgaccaaacagctggctgaaatcccggcgtatatgcaaaccctggataacggggtcgagtacctgttgagtcgtcaacatgaggaaggctattggtggggtccgcttctctcgaatgtaactatggaagcggaatatgttctgctgtgccattgcttgggaaaagttgataaagaacgcctggagaaaatcaaaacgtacttactgcacgaacaacgcgaagatggaacttgggctcagtacccaggtggtccgcaggacctggacacgactatcgaagcgtacgtggcactgaaatatattggtttatcgcctgacgacgaacgtatgcagaaagcgctggctttcatccagagtcagggtggcattgaatcggcgcgggtgtttacccggctctggctggcactggtaggggaatacccatggcgtaaactgccggttgtgccacccgaaatcatgtttctcgggaagaacatgcctttaaatatttatgacttcggcagctgggctcgtccaactatcgttgcccttactatcgttatgagtcggcgggcggtgtttcccctgccagcccatgcgaaagttcccgagttattcgaaacgaacgttccgccacgccgtcgtgccgcgaaaggcggcaacagtagtctgtttctgtccatcgacaaactgctgcaaggataccagaacggctccttccatccattccgcaaagccgctgagcaacgcgcgatcgagtggctaatcgaacatcaagccggtgatggtagctggggcgggatccagccgccgtggttttacgcactgttggctcttaaagtgatgaacatgacctcgcatccggcctttattaaaggctgggaaggtctggagttatatggcctggaactggaatacggtgggtggatgtttcaggcgtctatctccccggtgtgggataccggcctgagcatccttgccctgcgcgccgcgggactcgcccctgatgaaccggcgcttgtcaaagcaggtcagtggctgctggatcatcgcatcgcaacaaaaggggattgggcagtgcgccgtccgaatgccaaacctggtggttgggccttccagtttgataatccccattatccggacgtcgatgatacggccgtggttgtctgggcccttaacggcctgaaactgcccaatgaggcggaacgtcgtgacgccatgacggccggttttcgttggctgaccgccatgcagtcgtctaacggtggctggggcgcgtatgatgtggataacaacaaagagttacctaatcgcatcccgttttgcgattttggcgaagtcattgatccgcctagtgaagatgttaccgcgcacgtccttgaatgtttcggttcctttggatatgacgaagcctggaaagttgttgcgcgtgctgtcaactacctgaaacgggaacagaaaccggacggctcctggtacggccgttggggcgttaattacatttatggtattggcgcggttgtcccggcattgaaatcggtcggagtggacatgaaagaaccgttcgtgcagaaagcgctagactggttggttgcacatcagaacgaagatggtggctggggtgaagattgtcgcagctatgtcgatgaacggtttgcgggcgtaggacccagcacacccagtcaaacggcctgggccctgatggcactgattgccggcggtcgtgtgcaagcggatgcagtgtcacgcggcgttgcgtatttggtgcgcacgcagcgtccggatggtggctgggacgaaccatattacaccggcactggttttcccggcgacttttatttaggctatactctttatcgccacatctttccggtcatggccctgggccgctacaaagatgctctgggccggcttacgcgt

SEQ ID NO: 5 - protein, 633 aa, unidentified
  source 1..633: note = Alicyclobacillus tengchongensis
MTKQLAEIPAYMQTLDNGVEYLLSRQHEEGYWWGPLLSNVTMEAEYVLLCHCLGKVDKERLEKIKTYLLHEQREDGTWAQYPGGPQDLDTTIEAYVALKYIGLSPDDERMQKALAFIQSQGGIESARVFTRLWLALVGEYPWRKLPVVPPEIMFLGKNMPLNIYDFGSWARPTIVALTIVMSRRAVFPLPAHAKVPELFETNVPPRRRAAKGGNSSLFLSIDKLLQGYQNGSFHPFRKAAEQRAIEWLIEHQAGDGSWGGIQPPWFYALLALKVMNMTSHPAFIKGWEGLELYGLELEYGGWMFQASISPVWDTGLSILALRAAGLAPDEPALVKAGQWLLDHRIATKGDWAVRRPNAKPGGWAFQFDNPHYPDVDDTAVVVWALNGLKLPNEAERRDAMTAGFRWLTAMQSSNGGWGAYDVDNNKELPNRIPFCDFGEVIDPPSEDVTAHVLECFGSFGYDEAWKVVARAVNYLKREQKPDGSWYGRWGVNYIYGIGAVVPALKSVGVDMKEPFVQKALDWLVAHQNEDGGWGEDCRSYVDERFAGVGPSTPSQTAWALMALIAGGRVQADAVSRGVAYLVRTQRPDGGWDEPYYTGTGFPGDFYLGYTLYRHIFPVMALGRYKDALGRLTR

SEQ ID NO: 6 - DNA, 2160 bp, Acetobacter pasteurianus
  source 1..2160: mol_type = genomic DNA
atgaatatggcgtcacgtttctcgctcaagaagatcctgcggagcggctcagatacgcagggtaccaatgttaacaccctgatacaaagcggcacatctgatattgtgcgccagaaaccggctccgcaggtacccgcggacctgtccgcattaaaagccatgggcaacagcttgacgcacaccctgtcaagcgcgtgtgagtggctgatgaaacagcagaaaccggatggccactgggttggcagtgttgggtctaatgcgagcatggaagcagagtggtgcctggccctgtggtttctgggccttgaagatcatccgctgcgtcctcgcctgggtaaggcactgttagaaatgcagcgtccggacggttcgtggggaacctattatggagcgggatcgggcgatattaacgctacggtagaatcatacgccgcactgcggagtctgggctatgcggaagatgatccggccgtttcgaaagccgccgcttggataatctcgaagggcggattgaagaatgtgcgtgtgtttacacgctattggctggcacttatcggtgaatggccgtgggagaaaaccccgaatctgccaccggaaattatctggttccctgacaattttgtgtttagcatctataactttgcgcagtgggcgcgtgcgacgatgatgccacttgccattctaagtgcacgtcgtccatcacgcccacttcgcccacaggaccgcctggatgcactattccctggtggtcgcgccaattttgattatgaactgccgaccaaagaaggcagggacgtaatcgcagatttcttccgtcttgctgataaaggtctgcactggctgcaaagttcatttctgaaacgtgcaccgtcacgtgaggcagccattaagtatgtgctagaatggattatctggcaccaggatgcagacggcggttggggcggtattcagccgccgtgggtatacggtctgatggcgctgcacggcgaaggctaccaatttcatcacccggtgatggccaaagcgctggacgcgctaaatgaccccgggtggcgtcatgataagggtgacgctagctggatccaagcgacgaacagccctgtttgggatactatgttgagcctgatggcgctgcacgacgcaaatgcggaagaacgctttaccccggaaatggataaagctctggactggctgctgtcacgccagatccgcgttaagggtgactggagcgtcaaactgcctaacacagaaccgggtggttgggcattcgaatacgcgaacgaccgctatccagacacggacgacaccgctgtggccctgattgcgattgcctcatgtcgtaataggccggaatggcaagcaaaaggcgtcgaagaagcgattggcaggggtgtacgttggctcgttgctatgcaatcctcgtgcggcggctggggtgcgtttgacaaagataacaacaagtcaatactggcgaaaattccgttctgtgatttcggtgaggccctcgacccaccgagcgtggacgttactgcacatgtcctcgaggccttcggtttgctcgggttgccaagagatttaccgtgcattcagcgcgggttagcgtacattcgtaaagaacaggatcctacgggcccgtggtttggccgatggggagtaaattacctgtatgggacgggcgctgtacttccagcgttggccgccctcggtgaggacatgacacaaccgtacatttccaaagcgtgtgattggctgataaactgtcagcaggaaaacggcgggtggggagaaagttgtgcgagctacatggaagttagctccatcgggcacggcgcaaccacgccttcacagaccgcctgggcgctgatgggcctcattgctgcaaatcggccgcaggactacgaagcgattgccaagggttgtcgatacttaattgatcttcaggaagaggacggcagctggaacgaagaagagttcaccgggacaggcttcccaggctatggcgtggggcaaaccatcaaactcgatgatcctgctatttctaaacgtctgatgcaaggcgccgaattgagccgcgcgttcatgctgcgttatgatctgtatcgccaactcttcccgattatcgccctctcacgcgcatcccgtctgatcaaactgggaaat

SEQ ID NO: 7 - protein, 720 aa, Acetobacter pasteurianus
MNMASRFSLKKILRSGSDTQGTNVNTLIQSGTSDIVRQKPAPQVPADLSALKAMGNSLTHTLSSACEWLMKQQKPDGHWVGSVGSNASMEAEWCLALWFLGLEDHPLRPRLGKALLEMQRPDGSWGTYYGAGSGDINATVESYAALRSLGYAEDDPAVSKAAAWIISKGGLKNVRVFTRYWLALIGEWPWEKTPNLPPEIIWFPDNFVFSIYNFAQWARATMMPLAILSARRPSRPLRPQDRLDALFPGGRANFDYELPTKEGRDVIADFFRLADKGLHWLQSSFLKRAPSREAAIKYVLEWIIWHQDADGGWGGIQPPWVYGLMALHGEGYQFHHPVMAKALDALNDPGWRHDKGDASWIQATNSPVWDTMLSLMALHDANAEERFTPEMDKALDWLLSRQIRVKGDWSVKLPNTEPGGWAFEYANDRYPDTDDTAVALIAIASCRNRPEWQAKGVEEAIGRGVRWLVAMQSSCGGWGAFDKDNNKSILAKIPFCDFGEALDPPSVDVTAHVLEAFGLLGLPRDLPCIQRGLAYIRKEQDPTGPWFGRWGVNYLYGTGAVLPALAALGEDMTQPYISKACDWLINCQQENGGWGESCASYMEVSSIGHGATTPSQTAWALMGLIAANRPQDYEAIAKGCRYLIDLQEEDGSWNEEEFTGTGFPGYGVGQTIKLDDPAISKRLMQGAELSRAFMLRYDLYRQLFPIIALSRASRLIKLGN

SEQ ID NO: 8 - DNA, 2166 bp, Phaeospirillum fulvum
  source 1..2166: mol_type = genomic DNA
atgacgaaccaacgcacgcggctcaaagtggcctcggaacgcgctgatgagattgatacagacggtgatcgaacggcaactcttccggtcagcccggttgtgaacagcggcaagaactcggcaccaatcggtttggccgcgaacaccgactctagcctcacggttaaaattaaagcagccattaacgcggctggagattggttgctggaccgccagaacgaggacggtcattgggtaggtccgttgcagagcaatgcatgcatggaagctcagtggtgtctggcgttatggttcctgggcttagagaaccaccctctccgccctcgcctcggacagtcactcctggaaacgcagcgtgaagatggctcatgggaagtctactatgctgcctctgcgggcgacatcaacacaaccgtggaagcgtacgccgcgctgcgttcccttggcttcccggaaagtgatccgcgtctgacgaaggcccgtgagtggattttgagcaagggcggactgggcaaagtacgtgtgtttactcgttattggctggcccttatcggcgaatggccatgggaacagaccccgaatctgccaccagaagtaatttggttaccggactgggccccgttctccatttataacttctcgcaatgggcgcgcgctacgatgatgccactggcggtactgagtgcgcgtcggccgagtcgccccttgccgccgggcaaccgtttggatgctttatttccggaaggtcgcgaacattttgattattctcttcctgttagagatggcgcgggttggggagatcgtttcttccgcgctgcggacaaggcactccatcgtttacaggacatgggtgcgcagtatggccgcttcgcaccgtggcgcgatgcagcggttcgccataccttggagtggattttgcgccatcaggacgcggatggcggatggggcggaatacaaccgccgtgggtatatggactgatggcgctgcacgttgaagggtatgcgctggaccatcctgtaatggcgaaaggcctcgaagcgctcgaccatccgggttggcgagtggataaaggtgaggcctcatggattcaagcgtccaacagccctgtatgggatacgatgcttacgctgatcgcgtttgatgatactggcctagccgaagcgcatcctgaagcgacggccaaagcggtgcagtggcttcttgatcaccagattcggcgtccgggtgactggtcgcgcaaattgccgggagtaaaaccgggtggatgggcgttcgagtatgcaaactcccagtacccggatatagatgatactgccgaagcgcttatcgcgctggcgccctttcgtcacgatcctgtatggcagacgagaggcattgaggaagcaataaccctgggcgttgattggctgataggtatgcaatctgcatcgggtggatggggcgccttcgataaggataacaataaacaactgctcacgaaaattccgttttgcgatttcggtgaagcgctagatccgccaagcgtggatgtgacggcccatattgtggaagcgctggcacgcctggggctctctgcggagcacccagctctggcgagagccctggcgttcatccgagcagagcaggaagccgatggcccctggttcggccgttggggtgttaattatatctatggcacttgcgccgtgctcccggccttagcgacgatcggcgaagatatgaaccagccctatgttggccgggcctgtgattggctggtcagtcgtcagcaagataatggtggttggggtgagtcttgcgcttcgtatatggacccagcccaggcgggatatggtcccgttactgcgtcgcagaccgcgtgggcgctgatggctttgctggcggttaaccgccctgaagatcgcgcagcaatcgaacgcggatgtcggtacctggtcgagcaccaggaaaacggttgttgggaagagccgcactataccgcaaccggtttcccgggctacggtgtgggtcagagcataaagctgaccgatcctgaaattgctcgtcggcttatgcaaggcagtgaattgtcgcgtgctttcatgcttcgatatgacttatatcgacattattttccgatgatggctctgggtcgcgcagtacgtagcggccgtgtgagcggtgca

SEQ ID NO: 9 - protein, 722 aa, Phaeospirillum fulvum
MTNQRTRLKVASERADEIDTDGDRTATLPVSPVVNSGKNSAPIGLAANTDSSLTVKIKAAINAAGDWLLDRQNEDGHWVGPLQSNACMEAQWCLALWFLGLENHPLRPRLGQSLLETQREDGSWEVYYAASAGDINTTVEAYAALRSLGFPESDPRLTKAREWILSKGGLGKVRVFTRYWLALIGEWPWEQTPNLPPEVIWLPDWAPFSIYNFSQWARATMMPLAVLSARRPSRPLPPGNRLDALFPEGREHFDYSLPVRDGAGWGDRFFRAADKALHRLQDMGAQYGRFAPWRDAAVRHTLEWILRHQDADGGWGGIQPPWVYGLMALHVEGYALDHPVMAKGLEALDHPGWRVDKGEASWIQASNSPVWDTMLTLIAFDDTGLAEAHPEATAKAVQWLLDHQIRRPGDWSRKLPGVKPGGWAFEYANSQYPDIDDTAEALIALAPFRHDPVWQTRGIEEAITLGVDWLIGMQSASGGWGAFDKDNNKQLLTKIPFCDFGEALDPPSVDVTAHIVEALARLGLSAEHPALARALAFIRAEQEADGPWFGRWGVNYIYGTCAVLPALATIGEDMNQPYVGRACDWLVSRQQDNGGWGESCASYMDPAQAGYGPVTASQTAWALMALLAVNRPEDRAAIERGCRYLVEHQENGCWEEPHYTATGFPGYGVGQSIKLTDPEIARRLMQGSELSRAFMLRYDLYRHYFPMMALGRAVRSGRVSGA

SEQ ID NO: 10 - DNA, 2154 bp, unidentified
  source 1..2154: mol_type = genomic DNA; note = Komagataeibacter diospyri
atgacccgagagagtcgcccgttgaccaaaaccgcgtcgagttctgatgctgtgattcacgatattgcttcaggcaccacctttccggcggccggcatccagtccgtcaaggggaaagacaagagtttgccggctgccagcgctctgcgcaccatggacaactcgttgagccatgccatcagtagcgcgtgcgattggctggtaggccaacagaaaccggatggtcactgggtgggcccggttgcaagtaacgcgagcatggaagcggaatggtgcttagcgctctggttcttgggcctcgaagatcacccgctgcgcccgcgcctgggcaaagccttactggagatgcagcgcgaagatgggagctggggtacgtattatggtgctggcaacggtgatattaacgcgaccgttgagtcgtatgcggcgctgaggtcccttggctatccagctgatgatccggccgtgtcgcgcgcggcgacctggatcgcgagcaaaggcggattgaagaatattcgcgtgttcacgcgttattggctggcgttaattggtgagtggccgtgggagaaaacgccgaacttaccgccggaagtcatttggttccccaataaattcgtattctcgatatataactttgcgcagtgggcgcgtgccaccctggttccgttggccattctgtcagcgcggcgcccttcgcgcccattgcgaccgcaggatcgtttagatgccttatttccgggtggccgcgctaacttcgactatgaactgccagcgcgtggcgaacgcgatctgtgggatcgcttcttccgtaccaccgatcgtggcttgcactggctccaatcccgcttcttgaaacgcaacacattgcgcgaggcagcaatccgtcacatgctggaatgggtgattcgtcatcaggatgccgacggtggatggggcggcatccagccgccatgggtttatggtctgatggcactgcacggcgaggattaccagttccatcacccggttatggcgaaagccctaagtgcactgaacgaccccggctggcgtcacgaccgtggtgacgcgtcctggatacaagcaaccaattctccagtctgggacaccatgctggctatcatggccctgcatgacgccgacggcgaaacccgtttctcaccgcagatggagaaggctctcggctggctgctcgatcgtcaggtgcgagtgaaaggagattggtcgattaaactgccggatgtggaaccaggtggctgggccttcgaatacgccaacgatcgataccccgacactgatgacacggctgtagcgctgatcgctttaagcagctgccgcaaccgcgaggaatggaagaaacggggtgtcgaagatgctatccgccgcggggtcaattggctgatcggtatgcaaagcgaatgtggcggttggggtgccttcgataaggacaacaaccgcagtattctcagtaaaattccgttctgcgactttggcgaagcactggacccgccgtcggtagatgtaacagcgcacgtactggaagcgttcggggtgctgggtctgccgcgtgatctgcccgccatccagcggggcctggcctatattcgtgcagaacaggaaccggatggcccgtggttcggacgttggggagtgaattatttatatggtaccggtgctgtgctccctgctttggcagccatcggcgaggacatgacgcaaccgtatatcacgcgcgcgtgtgattggctggttgcgcatcaacaggagaacggcggctggggagaatcgtgtgccagctacatggagttgtccgccgtagggcgcggcccgaccaccccgagtcagacggcgtgggcgctgatgggtctgattgccgcgaatcgtcctcaggattatggtgcgatcgcccgtgggtgtcgctatctgatcgatctgcaacaagccgatgggtcctggcatgagaaagaatttactggcacgggtttccccggatacggggttggtcagacgatccggttagatgatccagccctttccaaaagacttcagcagggagccgaactaagtcgtgcattcatgctgcgatacgatctctacaggcagttcttcccgataatggcactaagtcgcgcaagtcgtctgatgaaattagaaaag

SEQ ID NO: 11 - protein, 718 aa, unidentified
  source 1..718: note = Komagataeibacter diospyri
MTRESRPLTKTASSSDAVIHDIASGTTFPAAGIQSVKGKDKSLPAASALRTMDNSLSHAISSACDWLVGQQKPDGHWVGPVASNASMEAEWCLALWFLGLEDHPLRPRLGKALLEMQREDGSWGTYYGAGNGDINATVESYAALRSLGYPADDPAVSRAATWIASKGGLKNIRVFTRYWLALIGEWPWEKTPNLPPEVIWFPNKFVFSIYNFAQWARATLVPLAILSARRPSRPLRPQDRLDALFPGGRANFDYELPARGERDLWDRFFRTTDRGLHWLQSRFLKRNTLREAAIRHMLEWVIRHQDADGGWGGIQPPWVYGLMALHGEDYQFHHPVMAKALSALNDPGWRHDRGDASWIQATNSPVWDTMLAIMALHDADGETRFSPQMEKALGWLLDRQVRVKGDWSIKLPDVEPGGWAFEYANDRYPDTDDTAVALIALSSCRNREEWKKRGVEDAIRRGVNWLIGMQSECGGWGAFDKDNNRSILSKIPFCDFGEALDPPSVDVTAHVLEAFGVLGLPRDLPAIQRGLAYIRAEQEPDGPWFGRWGVNYLYGTGAVLPALAAIGEDMTQPYITRACDWLVAHQQENGGWGESCASYMELSAVGRGPTTPSQTAWALMGLIAANRPQDYGAIARGCRYLIDLQQADGSWHEKEFTGTGFPGYGVGQTIRLDDPALSKRLQQGAELSRAFMLRYDLYRQFFPIMALSRASRLMKLEK

SEQ ID NO: 12 - DNA, 2079 bp, Acetobacter orleanensis
  source 1..2079: mol_type = genomic DNA
atgaccaccccattgtttaaaggaatgggcaactcgctgacccacaccgtcagctctgcgtgcgaatggctgatttctcaacaaaatccggacggtcattgggtgggacctgtcggaagtaacgcttccatggaagctgaatggtgcctggctctttggtttcttggcctagaggaccatccactgcgaccgagactcgggaacgcgttgttacaaactcagcgtgaagatggttcatgggacgtctacctgggcgcaggcaatggtgacattaatgccacggtcgaagcctatgctgcgttacggagcctgggctatccggagaacaccccggcccttcagaaagcggctacctggatcaaacagaaaggtgggctgaagaatatccgggtgtttacacgttactggctggcgctgatcggcgaatggccatgggaaaagacgccgaatcttccgccggaaatcatctggttcccgaacaaatttgttttcagcatctataacttcgcccagtgggcccgtgcgaccctggttccgctggcgattttatccgcccgtcgtccgtcacgcccactgcgtcctcaggaccgcctaaacgccctgtttccagagggacgcggtaactttgattatacgttaccgaagaaggaaggcgtggatctgtggtcggacttctttcgtacaaccgacaaaggcttgcactggttgcaatccaaattccttaaacggaacacgatgcgtgaggcagccatccgtcacatgttagaatggataatccgccaccaggatgcagatggcggctggggcggtatccagccaccctgggtttatggccttatggccctgcacggcgaaggataccagtttcatcacccggtgatggcaaaaggtctggacgcactgaacgatccaggctggcggcacgacaaaggtaacgctagctggatccaagctacgaatagtcctgtctgggatacgatgctggcaattatggcactgcacgacgccaaagcggaagatcgcttcacaccgcaggttgacaaagccctggggtggctgcttgatcgccaagttcgtgtgaaaggggattggagcattaagttgcctgatgtcgaacctggcggttgggccttcgaatatgcaaacgacttctatcctgacactgatgacacggcggtggcacttattgcgctggcgagttgccggcatcgtccggaatggcaagaacgtggcgtagaggatgcgattgctcgcgccgtacgttggctggtggccatgcagtcgtcttgtggtggctggggagccttcgataaagataacaataaggcgttgttgagtaaaatacccttttgtgatttcggcgaagcactcgatccaccttcagttgacgtaacggcacatatcctggaagctttcggtttgctgggccttccgcgcgatctgccgtgtatcaaacatgcgctggattacgtgcgcgctgagcaagatcctcaaggcccttggtttggtcgctggggtgttaattatgtatatggcacgggcgcggtgctgcctgcgttagctgcaatcggtgaagatatgactcaaccgtatattaccaaagcgtgcgattggttagttgcgcatcagcaggaagatggcggctggggtgaatcttgcgccagttacatggacgcctcaaccattggtagaggcaaaaccacaccgtcacaaaccgcatgggctctgatgggactgatcgcagccgcgcgtccgcaggactatccggccattgaaaagggatgtcgctacctcatcgatcgtcaggaacctgatggctcctggacggaagaagattataccggcaccggctttccggggtatggcgtgggtcaaacgattcgtctggacgatcctgccctgtccaaacgtttacagcagggtgctgaactgtcccgcgcctttatgttgcgctatgatttatatcgccagttctttccgattatggcgctctctcgcgcgtcaaggctgattagtccggaaaccgccaccgaacaagctgtagaggcggcagcgaagaacctggagaaaatcattgct

SEQ ID NO: 13 - protein, 693 aa, Acetobacter orleanensis
MTTPLFKGMGNSLTHTVSSACEWLISQQNPDGHWVGPVGSNASMEAEWCLALWFLGLEDHPLRPRLGNALLQTQREDGSWDVYLGAGNGDINATVEAYAALRSLGYPENTPALQKAATWIKQKGGLKNIRVFTRYWLALIGEWPWEKTPNLPPEIIWFPNKFVFSIYNFAQWARATLVPLAILSARRPSRPLRPQDRLNALFPEGRGNFDYTLPKKEGVDLWSDFFRTTDKGLHWLQSKFLKRNTMREAAIRHMLEWIIRHQDADGGWGGIQPPWVYGLMALHGEGYQFHHPVMAKGLDALNDPGWRHDKGNASWIQATNSPVWDTMLAIMALHDAKAEDRFTPQVDKALGWLLDRQVRVKGDWSIKLPDVEPGGWAFEYANDFYPDTDDTAVALIALASCRHRPEWQERGVEDAIARAVRWLVAMQSSCGGWGAFDKDNNKALLSKIPFCDFGEALDPPSVDVTAHILEAFGLLGLPRDLPCIKHALDYVRAEQDPQGPWFGRWGVNYVYGTGAVLPALAAIGEDMTQPYITKACDWLVAHQQEDGGWGESCASYMDASTIGRGKTTPSQTAWALMGLIAAARPQDYPAIEKGCRYLIDRQEPDGSWTEEDYTGTGFPGYGVGQTIRLDDPALSKRLQQGAELSRAFMLRYDLYRQFFPIMALSRASRLISPETATEQAVEAAAKNLEKIIA

SEQ ID NO: 14 - DNA, 2118 bp, unidentified
  source 1..2118: mol_type = genomic DNA; note = Komagataeibacter rhaeticus
atgacctttctgactggtaccgagaaaatgaacaaagaaagtcgcccgttcccgccgggtacgaccggcccagttagtggtaaagcgcctcaggcgacggacggttctttccgcgcgctagataattcactgtctcacgcgttatccgcggcctgcgattggctgattggccagcagaaaccggatggtcattgggtgggcccggtcgctagcaacgccagtatggaagccgagtggtgcctcgccctgtggttcttgggtctggatgatcatccgctacgcccgcgcctcgggcacgcgctactcgaaatgcagcgtgaagatggatcgtggggcatctactacggcgcgggcaatggagatattaatgcgaccgttgaatcttacgcggctcttcgctcgcttggctatgcagcggacgaaccggccctagcgaaagccgcaacttggattgcaagcaaaggtggtttgcgtaacatccgcgtattcactcgctactggctggcactgatcggtgaatggccgtgggagaaaaccccgaatctgccgcctgaggttatttggtttcctaataaattcgttttcagcatttacaactttgcccaatgggcccgggcaaccttagtgcccctagctatcctttcagctcgccgcccggcacgcccattacgcccgcaagaccgcttggatgcgctgttcccaggtggccgcgcgaattttgattatgaacttccgcgaaaagaaggtcgtgacctttgggctagcttcttccgcaccaccgaccgtggcctgcactggttacagagccgcgttatgaagaagtcgagtctgagagaagctgcaatccgtcacatgctcgagtggataatccgtcatcaggacgccgacggcggctggggcggtattcaaccgccttgggtctatggcttaatggcgttacacggtgaggattatcagtttcatcacccagtgatggcaaaaggcctggcggcgttagacgatccgggatggcgccacgaccgcgggcaggcgtcctggattcaggccaccaacagtccggtatgggacacgatgcttgcaatcatggcgcttcatgatgccgatggcgaaacccgtttcaccccagaaatggatcgcgcactgggctggttgctggatcgtcaggtccgcgtgaagggtgactggtcaattaagctgccggatgtcgaaccaggtggctgggcgtttgagtatgcaaacgaccgatacccggatactgatgatacagcggtggcccttattgcgctgtcgtcgtgccgtaatcgtccagagtggcaggcacgcggggtagaaccggcaattaaacgtggggtaaattggcttgtggccatgcagtcagagtcaggtggctggggcgcgtttgataaagataacaatcgctctttgttggctaaaattccgttctgcgattttggcgaagccctggatccgcctagcgtggacgttacggcccatgtgctagaagcattcggtgtgttaggcttaccgcgcgacatgccagcgattcgccgtggcctggcctatattcgtgcggaacaggctgcggacgggccttggtttggtcgttggggtgttaactatctgtacggtaccggtgccgtcctgccggcccttgccgctatcggcgaggatatgacgcagccgtatattacgcgtgcttgcgattggcttgtaggatgtcaacaagcagatggcgggtggggagaaagctgtgcctcatatatggagattgcggcgattggtcatgggcctacaacaccatcacagactgcatgggcacttatgggtctgattgcggctcatcgtcctcaagatcacgccgctatcgctcgcggctgccgttacctaattgaccttcagcaaccggatggccgctgggatgaaaaggagtttacgggtaccggattcccgggttacggtgtcggtcaaaccattcggttagacgatccggccctgtcgcagcgcttacagcaaggcgccgagctgtctcgtgcttttatgctgcgttacgatctctatcgtcaattcttcccgatcatggctctgtcccgggccgcacgcgtgctcaaagcggctgga

SEQ ID NO: 15 - protein, 706 aa, unidentified
  source 1..706: note = Komagataeibacter rhaeticus
MTFLTGTEKMNKESRPFPPGTTGPVSGKAPQATDGSFRALDNSLSHALSAACDWLIGQQKPDGHWVGPVASNASMEAEWCLALWFLGLDDHPLRPRLGHALLEMQREDGSWGIYYGAGNGDINATVESYAALRSLGYAADEPALAKAATWIASKGGLRNIRVFTRYWLALIGEWPWEKTPNLPPEVIWFPNKFVFSIYNFAQWARATLVPLAILSARRPARPLRPQDRLDALFPGGRANFDYELPRKEGRDLWASFFRTTDRGLHWLQSRVMKKSSLREAAIRHMLEWIIRHQDADGGWGGIQPPWVYGLMALHGEDYQFHHPVMAKGLAALDDPGWRHDRGQASWIQATNSPVWDTMLAIMALHDADGETRFTPEMDRALGWLLDRQVRVKGDWSIKLPDVEPGGWAFEYANDRYPDTDDTAVALIALSSCRNRPEWQARGVEPAIKRGVNWLVAMQSESGGWGAFDKDNNRSLLAKIPFCDFGEALDPPSVDVTAHVLEAFGVLGLPRDMPAIRRGLAYIRAEQAADGPWFGRWGVNYLYGTGAVLPALAAIGEDMTQPYITRACDWLVGCQQADGGWGESCASYMEIAAIGHGPTTPSQTAWALMGLIAAHRPQDHAAIARGCRYLIDLQQPDGRWDEKEFTGTGFPGYGVGQTIRLDDPALSQRLQQGAELSRAFMLRYDLYRQFFPIMALSRAARVLKAAG

SEQ ID NO: 16 - DNA, 2055 bp, Acetobacter malorum
  source 1..2055: mol_type = genomic DNA
atgggcaacagcctgacgcacactgtttcaagtgcgtgtgagtggctgataagccaacagaaacccgacggtcactgggtagggcctgtgggatctaacgcgagcatggaagcagaatggtgccttgcgctatggttcctgggcctggatgatcaccccttgcgtccgcgcttgggcaacgcgttgttgcagacgcagcgtgatgatggatcatggggtatctacctcggcgcaggtaatggtgatattaatgccaccgttgaagcgtatgcggccctgcgttctctgggttatcccgaagatcatccggctttacataaagcatctgcctggattgcccagaaaggcggcctgcgcaacatccgcgtctttactcgttactggcttgcgctgattggcgaatggccctgggaaaagactccgaacttgccgccagaaatcatctggtttccgaataaattcgtctttagcatatacaacttcgcgcagtgggcacgcgcaaccctggtgcccttggctgtgatcagcgcacgtcgcccatcccgcccgctgaggccacaggataggctgaacgcactgtttcccgagggtcgcgccaacttcgattataccctgccgaagaaggaaggcagagacttatggagtgacttctttcgtacgacggacaagggtctgcactggctgcaaagcaagtttctgaaacgtaataccttacgtgaggcagcaattcgccacatgttagaatggattattcgtcatcaggatgcggacggtgggtggggcgggattcaaccgccttgggtgtacggccttatggccctgcatggcgaaggtttccagttccatcatcctgtaatggctaaagcgctggacgcgctgaacgatccgggctggcgccacgataaaggtgacgcgagctggattcaagcaacaaatagccccgtatgggataccatgctggcaattatggccctgcatgatgccaaagcagaggaacgcttcaccccgcagatggataaagcgctgggttggttgctcgatcgccaggttcgcgtcaaaggcgattggagcatcaaactgccggatgtggaacccggtggctgggctttcgaatatgcgaacgatttctacccggatacagacgatacggcagtcgcactgattgcgctagcatcctgtcgccaccgtccggagtggaaagaacgtggagttgaagaggcgatcgcgcgggccgtccggtggctggtaggcatgcaatcgagttgcggtggctggggtgcctttgataaagacaataataaaactcttctgtcgaagattcctttctgtgatttcggcgaagctttagacccgccaagcgttgatgttaccgctcatatccttgaggcttttggtattttaggactgcctcgtgacctgccgtgcattcagcgcgcgcttgcctatgtccgcgccgaacaagatccacagggcccgtggttcggtcgttggggcgtgaattatgtgtacgggaccggtgctgtgctgccggccctggctgcaatcggtgaggacatgacccagccctacatcactaaagcctgtgactggctggctgcccatcagcaggatgacggcggttggggcgaatcttgcgcttcatatatggaagtttccgccattggtcggggtaagactaccccaagtcagaccgcctgggcactgatgggtttaattgccgcggcccgtccgcaagatttcgcggcgatcgagaaagggtgtcgttatctgattgatcgtcaagaaccggatggctcgtggactgaagaggaatacaccggtaccggctttccgggttatggtgtggggcaaaccattaaactcgacgatccgacgctgagtaagcgcttattgcaaggtgccgagcttagccgcgccttcatgctccgctacgacctgtaccgtcaattctttccgatcatggctctgtcccgcgcgtcccgtcttattacaccgaatgcgactgcggagaaagcaatcaacgcggcgcgtaagaatctggcggaaaccgttgcg

SEQ ID NO: 17 - protein, 685 aa, Acetobacter malorum
MGNSLTHTVSSACEWLISQQKPDGHWVGPVGSNASMEAEWCLALWFLGLDDHPLRPRLGNALLQTQRDDGSWGIYLGAGNGDINATVEAYAALRSLGYPEDHPALHKASAWIAQKGGLRNIRVFTRYWLALIGEWPWEKTPNLPPEIIWFPNKFVFSIYNFAQWARATLVPLAVISARRPSRPLRPQDRLNALFPEGRANFDYTLPKKEGRDLWSDFFRTTDKGLHWLQSKFLKRNTLREAAIRHMLEWIIRHQDADGGWGGIQPPWVYGLMALHGEGFQFHHPVMAKALDALNDPGWRHDKGDASWIQATNSPVWDTMLAIMALHDAKAEERFTPQMDKALGWLLDRQVRVKGDWSIKLPDVEPGGWAFEYANDFYPDTDDTAVALIALASCRHRPEWKERGVEEAIARAVRWLVGMQSSCGGWGAFDKDNNKTLLSKIPFCDFGEALDPPSVDVTAHILEAFGILGLPRDLPCIQRALAYVRAEQDPQGPWFGRWGVNYVYGTGAVLPALAAIGEDMTQPYITKACDWLAAHQQDDGGWGESCASYMEVSAIGRGKTTPSQTAWALMGLIAAARPQDFAAIEKGCRYLIDRQEPDGSWTEEEYTGTGFPGYGVGQTIKLDDPTLSKRLLQGAELSRAFMLRYDLYRQFFPIMALSRASRLITPNATAEKAINAARKNLAETVA

SEQ ID NO: 18 - DNA, 2034 bp, Rhodoblastus acidophilus
  source 1..2034: mol_type = genomic DNA
atgggcgaggcgctcacagtcgcggataccccggtttcacaagacaactttgccgccgctttgcgctccggcgtgcgcgccgcagttgattggctggttgagcgccagaaaccagacggtcattgggtcggccgcgccgaaagtaacagttgtatggaagcgcagtggcgcctggcactgtggttcctcggcttggacgatcacccgttatgtgcccgcctggcgcaaagcctgttggacacccaaagacctgatggagcttgggaaatttattacggcgcgccgaatggggacatcaatacaacagtcgaagcttatgccgcgttacggtgctccggatatgcggcgggtcacccagcattattgaatgcccaggcctggatcgagagtaagggtggactgaagaacattcgtgtgtttacgcgctattggctggcgctgattggtgagtggccgtgggaaaagacccctaacgtaccgcctgaagtcatctggtttccgctctggtttccgttcaatatttatcatttcgctcaatgggcacgggctacacttgttcccattaccgttttgtcggcacggcgtccgtcccgtccgctgccgccgcagaaccggctggatgcactctttccggaaggccgtgacgcgtttgattacgcgcttccggcgaaaccgcagccggatttgtgggaccgcttctttcgtcgcgctgacaaaatcctgcatggcttacaagatttcggtcatcttacaggtttaggtggtttgcgttcagcagcggtgcgccatgtattggaatggatcgtcaaacaccaggacgccgacggggcctggggtggcatccagccgccgtggatttatagcctgatggccttgaaaacggaaggctatcctgttacgcacccagttctggccaaagggctagatgcactgaacgatccgggatggcgcgtcgatgtgggtgatgcaacgttcatccaagcgaccaactcccctgtctgggatacgatgttgactcttctggcgtttgacgacgctgaagctctggactcggaagccgccgataaagcggttgactggctgctgcgccgtcaagtgcgggttccgggcgattggtccgtcaaactgcctaaagttcagcctggcggctgggcttttgaatatgctaacaactgctacccggataccgacgacacagcggtggcattgatagccctagttccgtttcgtacccaggaaaagtggaaggcgcgtggcattgaagaagcgattcagttaggcgtggattggctgatcgcgatgcagtctcagtcgggcggctggggtgcctttgacaaagataatacccggaaaattcttaccaaaataccgttttgcgatttcggcgaagctcttgatccgccgagtgtcgatgtgacggcacacgtggtggaagcgtttggcaaactgggtatatcccgggagcacccgagtatgattagagcgttacgctacattcgtgaacaacaggaagcggatggaccgtggtttggccgctggggagtcaactacctgtatgggacgggcgcggtgttgccagcgcttgcagcaatcggcgaggatatgacccagccgtatattggccgcgcctgcgattggctggttagtcgccagcaggccaatggtggctggggtgaaagctgtgcgtcatatatgcaagcagatcaggcggggcggggcgaagcaactgcatcgcagacaggatgggcgttgatggccctgctggcggtgggccgtgcccaagatcgcgaaagcatcgaacgcggttgccaatatcttctggaaaatcagcgtgagggcacctggcctgagcctcactttaccggtaccggcttcccgggctacggtgtgggccaaactatccgtctggatgacccactcttggcacagaggttgaagcagggccccgagttgagccgcgcgttcatgctgcgctacggcatgtattgccactacttcccgctgatggcgttatcccgcgccattcgtcgcatc

SEQ ID NO: 19 - protein, 678 aa, Rhodoblastus acidophilus
MGEALTVADTPVSQDNFAAALRSGVRAAVDWLVERQKPDGHWVGRAESNSCMEAQWRLALWFLGLDDHPLCARLAQSLLDTQRPDGAWEIYYGAPNGDINTTVEAYAALRCSGYAAGHPALLNAQAWIESKGGLKNIRVFTRYWLALIGEWPWEKTPNVPPEVIWFPLWFPFNIYHFAQWARATLVPITVLSARRPSRPLPPQNRLDALFPEGRDAFDYALPAKPQPDLWDRFFRRADKILHGLQDFGHLTGLGGLRSAAVRHVLEWIVKHQDADGAWGGIQPPWIYSLMALKTEGYPVTHPVLAKGLDALNDPGWRVDVGDATFIQATNSPVWDTMLTLLAFDDAEALDSEAADKAVDWLLRRQVRVPGDWSVKLPKVQPGGWAFEYANNCYPDTDDTAVALIALVPFRTQEKWKARGIEEAIQLGVDWLIAMQSQSGGWGAFDKDNTRKILTKIPFCDFGEALDPPSVDVTAHVVEAFGKLGISREHPSMIRALRYIREQQEADGPWFGRWGVNYLYGTGAVLPALAAIGEDMTQPYIGRACDWLVSRQQANGGWGESCASYMQADQAGRGEATASQTGWALMALLAVGRAQDRESIERGCQYLLENQREGTWPEPHFTGTGFPGYGVGQTIRLDDPLLAQRLKQGPELSRAFMLRYGMYCHYFPLMALSRAIRRI

SEQ ID NO: 20 - DNA, 2040 bp, unidentified
  source 1..2040: mol_type = genomic DNA; note = Amphiplicatus metriothermophilus
atgttggatgagcaagcagccaaagccgcccggggcgataatttccaagccgcgttgtcagaggcaatccaggcctcggcagattggttggtccgccggcagaaaccggaaggttactgggtgggccgcctggagtcaaatagttgtatggaagcggagtggcgtttggcgttgtggtttatggggttagatgatcaccacttgtgtccgcgcctggcagcgtcgctgcgggaatcgcagtctgcggatggtagctggcgtatttatcacggagcgccggcaggggatattaataccaccgtggaagcgtatgcggccctgcgctgtcacggggacgatccgcacgcccctcacatggcgagggcgcgcgcgtggatcctgtcaaaaggtgggctgcgtaatgtccgcgtgttcacgcgttattggctggcgttaattggtgagtggccgtgggatcgtaccccgaatctcccgccggaagtcatttggttccctaattggttcgttttctccatttataactttgcacaatgggctcgtgctacacttatgccaattgcggtcctgtctgcccgccgcccgacccgccctctcccggcagaacggcgtctaaatgaactgtttccggaaggtcgggctcgttttgattacgagcttccgcctaaaccgggcgctgacttttgggatcgcttcttccgtgcagctgataagaccctgcacgcattacagggctttggtcggaaaactggcctgtccctggggcgggatgcggcgattaaacgcgcgctggaatggatcatccggcatcaggacgcagatggcgtctggggtggcattcagccgccatggatctattcactgatggcacttcataccgaagggtatgctttagatcatccggtgctggctaaaggactggcggcgctggatgatccgcggtggagagtggatgtaggcgaagcgacctggatccaagcttcgacgagtccggtctgggacacggagcttactctattggctttacacgatgcggatgtcgtcaaagcccacgaagccgcggttgaacgcgcggtgcaatggctcttagatcaacaggttagacgtccgggcgattggcgtgtaaaagtgccgaacgtggaaccaggcggttgggcatttgaatacaagaattacttctacccggataccgatgataccgccgtcgccctgattgctctcgcaccgtttcgtgatgatccgaaatggaaggcgaaaggcttggacgaggcgattcgtcttggggttgattggctgatcggtatgcagtctgaatgcggcggttggggcgcctttgacaaagacaatgataagaaaattcttaccaagatccccttttgtgatttcggcgaggcgctcgatccaccaagtgtagacgtgaccgcccatgtgattgaagcgtttggtaaactgggctacgacaaaacacacccggcgatgaaacgcgccctggattggatgaagaaggagcaggaaccggatggttcttggtttggccgctggggtgtgaactatgtttatggtactggggcagcattaccggctctggaagctatcggcgaagatatgtctgcaccgtatgtacagcgtgcttgcgattggctggtttcaatgcagcaagcggacggtggttggggtgaatcttgcgcgtcctatatggatcctgcgatgaaaggtcgtgggacccctactgcgagccagactgcatgggcactgatggggttggttgcggccaaccgcgcgcaagatcgtgaagccatacagaaaggtctggcatttctagttgaacgccagaagaacggtacctgggaagagccggaatacactggcaccggttttccaggttacggtgtaggccagactattaaactcggcgatccgctccttgcagaacgccttaaacagggtccggaattaagtcgcgcatttatgattaactataacctctatcgccattactttccgcttatggccatgggacgcgcgcgcagactgcttggtgcc

SEQ ID NO: 21 - protein, 680 aa, unidentified
  source 1..680: note = Amphiplicatus metriothermophilus
MLDEQAAKAARGDNFQAALSEAIQASADWLVRRQKPEGYWVGRLESNSCMEAEWRLALWFMGLDDHHLCPRLAASLRESQSADGSWRIYHGAPAGDINTTVEAYAALRCHGDDPHAPHMARARAWILSKGGLRNVRVFTRYWLALIGEWPWDRTPNLPPEVIWFPNWFVFSIYNFAQWARATLMPIAVLSARRPTRPLPAERRLNELFPEGRARFDYELPPKPGADFWDRFFRAADKTLHALQGFGRKTGLSLGRDAAIKRALEWIIRHQDADGVWGGIQPPWIYSLMALHTEGYALDHPVLAKGLAALDDPRWRVDVGEATWIQASTSPVWDTELTLLALHDADVVKAHEAAVERAVQWLLDQQVRRPGDWRVKVPNVEPGGWAFEYKNYFYPDTDDTAVALIALAPFRDDPKWKAKGLDEAIRLGVDWLIGMQSECGGWGAFDKDNDKKILTKIPFCDFGEALDPPSVDVTAHVIEAFGKLGYDKTHPAMKRALDWMKKEQEPDGSWFGRWGVNYVYGTGAALPALEAIGEDMSAPYVQRACDWLVSMQQADGGWGESCASYMDPAMKGRGTPTASQTAWALMGLVAANRAQDREAIQKGLAFLVERQKNGTWEEPEYTGTGFPGYGVGQTIKLGDPLLAERLKQGPELSRAFMINYNLYRHYFPLMAMGRARRLLGA

SEQ ID NO: 22 - DNA, 2001 bp, Acetobacter sp.
  source 1..2001: mol_type = genomic DNA; note = DsW_54
atgggcaactccctcgctcacaccgtggccgcggcatgtgattggcttatcggcgagcagaaagcagacggacattgggttggtccagtagcgagtaatgcctcgatggaagcagaatggtgtctggccctgtggtacttgggcctggaagatcacccgttgcgtccgcgcttgggaaaagccctgttacacatgcaacgagaggatggttcttggggcacgtactggggtgccggcaatggagatataaatgcgactgttgaagcctatgccgccttgcgtagtctgggctatgcagcagacacgccggaactgtcaaaagcttgtgcgtggatcatgcgcatgggtggtctgcgtaatgttcgtgttttcacccgctattggctggcgctgatcggcgaatggccgtgggagcagaccccaaacttaccgcctgaagtgatttggttccctaacaaattcgttttctctatctacaattttgctcaatgggctcgcgctacattagtaccactggcgatcctgtcggctagacgcccgagccgtccgctgcgtcctcaggatcgcctggatgccctgtttccacaaggccgtgaaaacttcgactatgtgctgccgaagaaagaaggcgttgatctgtggtcgtccttctttcgcactactgataaaggtctgcattggttgcaaagccgtttcctgaaacgcaacacagtgcgtgaggctgcgattcgccacatgcttgagtggatcattcgccaccaagatgcggacggcggttggggcggcatccagccgccgtgggtatatggcttgatggccctgcacggcgaggattaccaatttcatcatccagtgatggcaaaagcgttagcggccctggatgaccccggttggcgtcgggaccagggtgatgcctcttgggttcaggcgacaaacagcccggtgtgggatacgatgctggcccttatggccctgcacgatgcgaacgctgaggaacgctacactccccagatggataaagccctcgattggctgctggcccgacaggtgcgcgttaaaggtgattggagtattaagttgccggatgtggaacctggcggctgggcatttgagtatgctaatgatcgctacccagacacggatgataccgctgtagcactgatcgccctgtccagctgccgcaatagagaagaatggaaagagaaaggcgtagaggacgcgattacccgcggtgtgaactggttgatcgcgatgcagagctcttgtggcggttggggtgcgtttgataaagacaataatcgtagcctattgagcaaaatccccttttgtgactttggggaagccctggatccgccgtcggtcgatgtgacggcccatgtactggaagcctttggcctgcttggcgtaccacgtcagactccagcgttacagcgaggtctggcttacattcgcgcagaacaggaagcttcgggcgcatggttcggtcgttggggtgtgaactatttgtatggaactggtgccgttctgcctgctctcgcagccattggcgaagatatgactcaaccctatattaccagagcttgcgactggctcatcgcgcatcagcaggaagatggcggctggggcgaatcttgtgccagctatatggacgtgtccagcattggctggggcacgacgacgccgtcccaaaccgcatgggccttaatgggtctgattgcggcaaatcgtgaacaggaccatccggcaattgcccggggctgccgttacctgatcgatcgtcaagaaacagatggttcgtggacggaagaagaatttaccggcacgggatttccaggctacggtgtgggccagacgattaagctcgacgacccggctgtcgcgaagcgcttgcaacagggagcagagctgagccgcgcgtttatgttgcgctatgatctgtatcgccaattcttcccgctgatggcactgagccgggcggctcgaattatgccggtcgggcaa

SEQ ID NO: 23 - protein, 667 aa, Acetobacter sp.
  source 1..667: note = DsW_54
MGNSLAHTVAAACDWLIGEQKADGHWVGPVASNASMEAEWCLALWYLGLEDHPLRPRLGKALLHMQREDGSWGTYWGAGNGDINATVEAYAALRSLGYAADTPELSKACAWIMRMGGLRNVRVFTRYWLALIGEWPWEQTPNLPPEVIWFPNKFVFSIYNFAQWARATLVPLAILSARRPSRPLRPQDRLDALFPQGRENFDYVLPKKEGVDLWSSFFRTTDKGLHWLQSRFLKRNTVREAAIRHMLEWIIRHQDADGGWGGIQPPWVYGLMALHGEDYQFHHPVMAKALAALDDPGWRRDQGDASWVQATNSPVWDTMLALMALHDANAEERYTPQMDKALDWLLARQVRVKGDWSIKLPDVEPGGWAFEYANDRYPDTDDTAVALIALSSCRNREEWKEKGVEDAITRGVNWLIAMQSSCGGWGAFDKDNNRSLLSKIPFCDFGEALDPPSVDVTAHVLEAFGLLGVPRQTPALQRGLAYIRAEQEASGAWFGRWGVNYLYGTGAVLPALAAIGEDMTQPYITRACDWLIAHQQEDGGWGESCASYMDVSSIGWGTTTPSQTAWALMGLIAANREQDHPAIARGCRYLIDRQETDGSWTEEEFTGTGFPGYGVGQTIKLDDPAVAKRLQQGAELSRAFMLRYDLYRQFFPLMALSRAARIMPVGQ

SEQ ID NO: 24 - DNA, 2067 bp, Rhodopseudomonas faecalis
  source 1..2067: mol_type = genomic DNA
atgactacacattcgacgatggcgcagcgggcgggcgcgtcttgtaatatggatggcgccttgcaggctaccattcagcaagcaactgagtggctcatgagtcagcagaaacccgacggccattgggtgggacggagcgagagtaatgcatgcatggaagcccaatggtgtctcgccctgtggttcattggtctggaagatcatcctctgcgtgttcgcctgggccaagccctgctcgatactcagcggcccgatggcgcgtggcatgttttccacggcgcgccgaacggagatattaacgccacggtggaagcttatgccgcgcttcgttctctgggtcaccgcgatgatgaacctgctctgcgcaaagcgcgggaatggatcctggctaagggtgggctgcgcaacatccgcgtctttacccgctattggctggcggtcattggggaatggccttgggacaaaacccctaatattccgcccgaagtcatttggctacccacttggttcccgctgtcaatttacaactttgcccaatgggcgcgtgcgaccctcatgccgatcgcgattctgagcgcccaccgtccatcccgcgcattgccgccggaaaatcgcctggatgcactgtttccggaaggtcgtgataacttcaattatgatctgccggaacggctgggcgcgggcgtgtgggacgcgttcttccgcaaagttgacacaatcctgcactcgttacagacctggggtgccaaacgtggccgtcatggcttaatgcgccgcgcagcattaaatcatgtactggagtggatcattcgtcaccaagatgccgacggggcgtggggcggtatccaaccaccatggatctatgggattatggcccttcattgcgaaggttacgcactgaaccatccggtcgtggcgaaagccctggactgtatgaatgatccgggctggcgtgtggacgtcggtgacgcgagtttcctgcaagccaccaacagtccggtgtgggacacccttctgtctttaatgtccctcgaggacgctgagatgcgtggtcaacatccggaagaggtagagcgctcggttcgctgggtgttacagcgccaggtccgtgtgccaggcgattggtcggttaaactgccaaatgtaaagcctggtggctgggccttcgaatacgcaaacaatttctatcctgacaccgatgacaccgcggttgcgttaatggccctggcgccatttcggaatgatccgaaatggcaagctgaagggattgaagaggccattcagttaggtgtcgattggctcataggtatgcagtgcaaaggcggtggttggggtgcgtttgataaggataataatcagaagctgctagctaaaatccccttttgtgatttcggtgaagccctggacccaccttctgcggatgtaaccgcgcatgtcatcgaagcgttttgcaagttaggcattgaccgccgacatccttcaatggcccgagcggttgcgtatttgaagcgcgaacaagaacccaacggtccatggttcggtcgttggggcgtaaattacctgtatggaaccggggctgtgctgccggctctggctgcgataggcgaggatatgtcccaaccgtatattggccgtgcatgcgattggttggtggcgcaacagcaggactcaggcggctggggcgagtcatgtagctcttacatggacccgagcgcagcgggtcgtggtgtggccacggccagtcagaccgcctgggcggtgatggctttactggctgccaatcgtccgaaggatcgtgacgcgatcgagcggggctgtttatttcttatcgataatcaacgtctgggtacttgggttgaacccgagtttaccggcaccggcttccctggctacggggtggggcagacgatcaaattaaacgatccgcttctgtcaaaacgcttgatgcagggtcctgaattgtctcgtagttttatgctccgctatgatatgtatcgccattattttccgctgacggccctggggcgcatgaaagcaatgctcgcaaaagaagcagccgctacccgt

SEQ ID NO: 25 - protein, 689 aa, Rhodopseudomonas faecalis
MTTHSTMAQRAGASCNMDGALQATIQQATEWLMSQQKPDGHWVGRSESNACMEAQWCLALWFIGLEDHPLRVRLGQALLDTQRPDGAWHVFHGAPNGDINATVEAYAALRSLGHRDDEPALRKAREWILAKGGLRNIRVFTRYWLAVIGEWPWDKTPNIPPEVIWLPTWFPLSIYNFAQWARATLMPIAILSAHRPSRALPPENRLDALFPEGRDNFNYDLPERLGAGVWDAFFRKVDTILHSLQTWGAKRGRHGLMRRAALNHVLEWIIRHQDADGAWGGIQPPWIYGIMALHCEGYALNHPVVAKALDCMNDPGWRVDVGDASFLQATNSPVWDTLLSLMSLEDAEMRGQHPEEVERSVRWVLQRQVRVPGDWSVKLPNVKPGGWAFEYANNFYPDTDDTAVALMALAPFRNDPKWQAEGIEEAIQLGVDWLIGMQCKGGGWGAFDKDNNQKLLAKIPFCDFGEALDPPSADVTAHVIEAFCKLGIDRRHPSMARAVAYLKREQEPNGPWFGRWGVNYLYGTGAVLPALAAIGEDMSQPYIGRACDWLVAQQQDSGGWGESCSSYMDPSAAGRGVATASQTAWAVMALLAANRPKDRDAIERGCLFLIDNQRLGTWVEPEFTGTGFPGYGVGQTIKLNDPLLSKRLMQGPELSRSFMLRYDMYRHYFPLTALGRMKAMLAKEAAATR

SEQ ID NO: 26 - DNA, 2127 bp, unidentified
  source 1..2127: mol_type = genomic DNA; note = Komagataeibacter medellinensis
atgaccttcttgactggtaccgaaaagatgaataaagaaagccgtccgttcccgcccaccgcaagcggcactgcgggtccggccgcgagcaccacccagcaagcacctacgggcagcccgcgtgctctggacaattcattatctcatacgatcagtgcggcatgcgattggctgataggacagcagaaaccagatgggcattgggtgggaccagtggctagcaatgccagcatggaagcagaatggtgtctggcactgtggttcctgggtctggaagatcatccgttacgaccgcgcttgggccatgctctgttagaaatgcagcgcgaggatggtagttggggcatttactacggcgcgggtaacggcgatattaatgccacagtggaaagctatgccgctctgcgctctttgggttatggagctgatgaaccgacattggcgcgcgcggccgaatggattgcatccaagggcggtctgcgcaatatccgtgtgtttacccgttattggctggccttgatcggtgaatggccgtgggaaaagacacctaatctgccgcctgaagtgatttggtttccaaattcgttcgtctttagcatctacaatttcgcacagtgggcacgcgcgaccctggtaccgctggccatcctgagtgcaagacgtcccgcgcgtccgctgcgcccgcaggatcgcctcgatgcccttttcccaggtggccgtgccaattttgactacgagctaccgcgtaaagaaggccgcgatttgtgggcgtcattcttccgtaccaccgatcgaggtctgcattggttacaatcacgtgttctcaagaagaactccgttcgcgaagcagcaattcgccacatgctggaatggattatccgccatcaagacgcagatggtggttggggtggtatacagccaccatgggtttatggcttgatggccttacatggtgaggattaccagtttcatcaccccgtaatggcgaaagccctgtcagctctgaatgatcccggctggcgtcacgaccgtggcgatgcaagttggattcaagcgacgaatagcccggtgtgggatactatgctggcactgatggcgctgcacgatgcggacggcgaaacgcgtttcacccctcagatggacaaagcgatggggtggttactggatcgccaggttcgagttaagggtgactggagcattaaattacccgatgttgaaccgggtggatgggcatttgaatacgcgaacgaccggtatcctgacaccgatgataccgcggtggcgctgattgccctttcgtcttgtcgtaaccggccggaatggcaggcgcgaggggtcgaagcagcaatcaagcgcggcgtgaactggttagttgcaatgcagtcagaatcgggtggctggggcgcgtttgataaggacaacaaccgcagtctgctggcgaaaattccgttttgcgacttcggcgaagcattggacccgccgtctgtagacgtaactgcgcatgtgctagaagcgttcggcctcttgggcttgccgcgcgatatgccggctattaggcgcggactcgcctacatccgtgcggaacaagctgcggaaggaccgtggtttgggcgctggggtgtgaattatttgtacggtactggtgctgtactgccggccttagccgcaattggcgaagatatgacccagccatacatcgcgaaagcgtgcgattggctggttggttgccagcaggaaaacggcggttggggtgaaagttgcgcgagctatatggagatttcgagtatcggtcgtggccctaccacgccgtcacaaacggcgtgggctttgatgggcctggttgcagccaatcgtaggcaggatcacgatgcgattgtgcgcggctgtcggtatctgatcgatctccagcagccggatggacggtgggacgagaaagaatttaccggcactgggtttccgggctatggcgtgggccagaccattcgtctggatgatcctgcgcttagcaaacgcttacagcagggagccgaactgagccgcgcgttcatgctgcgatacgacctgtaccgccagcttttccccatcatggcactgagccgcgcggccagggtgctgaaagccgcaggc

SEQ ID NO: 27 - protein, 709 aa, unidentified
  source 1..709: note = Komagataeibacter medellinensis
MTFLTGTEKMNKESRPFPPTASGTAGPAASTTQQAPTGSPRALDNSLSHTISAACDWLIGQQKPDGHWVGPVASNASMEAEWCLALWFLGLEDHPLRPRLGHALLEMQREDGSWGIYYGAGNGDINATVESYAALRSLGYGADEPTLARAAEWIASKGGLRNIRVFTRYWLALIGEWPWEKTPNLPPEVIWFPNSFVFSIYNFAQWARATLVPLAILSARRPARPLRPQDRLDALFPGGRANFDYELPRKEGRDLWASFFRTTDRGLHWLQSRVLKKNSVREAAIRHMLEWIIRHQDADGGWGGIQPPWVYGLMALHGEDYQFHHPVMAKALSALNDPGWRHDRGDASWIQATNSPVWDTMLALMALHDADGETRFTPQMDKAMGWLLDRQVRVKGDWSIKLPDVEPGGWAFEYANDRYPDTDDTAVALIALSSCRNRPEWQARGVEAAIKRGVNWLVAMQSESGGWGAFDKDNNRSLLAKIPFCDFGEALDPPSVDVTAHVLEAFGLLGLPRDMPAIRRGLAYIRAEQAAEGPWFGRWGVNYLYGTGAVLPALAAIGEDMTQPYIAKACDWLVGCQQENGGWGESCASYMEISSIGRGPTTPSQTAWALMGLVAANRRQDHDAIVRGCRYLIDLQQPDGRWDEKEFTGTGFPGYGVGQTIRLDDPALSKRLQQGAELSRAFMLRYDLYRQLFPIMALSRAARVLKAAG

SEQ ID NO: 28 - DNA, 2043 bp, unidentified
  source 1..2043: mol_type = genomic DNA; note = Marinicaulis flavus
atgttcgatgagaaagaagcaggcgctgataaacgttcgaatttcgatcgtcagttaacggcgtcgattgaggcagcagcagactggctcgctccccgacagaaaccggaaggctattgggttggtcggctggagtccaacgcttgcatggaagcacagtggatcttggcgttgtggtttatggaattagaagatcatcacctgcggccgagattagcagcgtcgctgcgcgaaacccaacgtcaggatggttcgtgggaaatctactacggtgctccggccggcgatattaacactacggtagaagcatacgcggcgctgcgttcaatgggcgatgacaaagatgcggcacacatggtccgtgcacgcgaatggatcttatcgaaaggcggcctgaagaacatcagagtctttacccgatactggcttgcgttaataggcgaatggccgtggaagaaaactccgaatctgccgcccgaagtgatcttcttcccgaattggttcgtgttttctatctataactttgcgcaatgggcccgcgccaccttgatgccgattgcggttctgagtgcacgtagaccaagccgcccgctgccagcggagcgtcgcctcgatgaacttttcccagaagggcgtgagaaatataactacgcctacccggagaagcctggtgccggcttcgccgaggcgttcttcctaacggcggacaaaatactgcatgctctgcaaagcttcggtcagacggcgggaatcggtctgctgcgcaaaggtgccatctcacgtgcgctggaatggattatcaagcatcaggatgcggatggggtgtggggcggcatccagccaccatggatctattctctgatggccctgtataacgaagggtatgcgcatgaccatagtgtggtggcgaaaggtctgggcgcccttgatgatccgcggtggcgtgttgatcagggcgaagccacgtggatccaggcatctacgagcccggtgtgggacactgaactgaccctattggcgctggaagattgtgacctatccgagcggcacgagaaagaggtggaaaaggccttggattggttgctgagccagcaggtactgcgcaaaggggactggagtgttaaatgcccgaagctggaaccgggcggttgggcgttcgaatataagaactactattatcctgacactgatgacaccgccgttgctctgatcgcgctcgctcccttccgtaatgatccgaaatggaaagataaaggcatcgaagaagccattgagaaaggagtggaatggctcatcggtatgcagtgcaagaatggtggttggggcgcgtttgataaagacaacgacaaacagattctgaccaaaatcccattctgtgattttggcgaggctctggatccaccaagcgtagacgtcaccgcccacatcatcgaagcgtttggtaagctgggcattaggaaagaccaccctgtaatgctgcgcgctctggactatatcaaaaccgagcaagaggcggacggggcgtggttcggccggtggggcgtaaattatgtgtatggtaccggtgctgtgctgcccgcgctggaagctatcggggaagatatgacccagcattatattaagaaggcttcggactggttgatcctgcaccagaatgaggatggcggctggggtgaatcctgcgcttcatacatggatgtcaaacagatgggtcgtggtaaatccacggctagtcaaacggcgtgggcactgatgggtcttgcggccgtcggtcgtgcggaagatgagcgtgcaattgcggacggtgtccaatttctgatcgagcgtcagaaagacggagcctgggaagagccggaatataccgggaccggcttcccgggatatggcgtgggtcagcatatcaaattgaccgatcctcagttgcaggagcggcttaaacagggtcccgaactgagccgcgcctttatgatcaactataacttgtatcgacattactttccgatgatggcaatgggccgcgtgcggaaaatgatggcaggcgct

SEQ ID NO: 29 - protein, 681 aa, unidentified
  source 1..681: note = Marinicaulis flavus
MFDEKEAGADKRSNFDRQLTASIEAAADWLAPRQKPEGYWVGRLESNACMEAQWILALWFMELEDHHLRPRLAASLRETQRQDGSWEIYYGAPAGDINTTVEAYAALRSMGDDKDAAHMVRAREWILSKGGLKNIRVFTRYWLALIGEWPWKKTPNLPPEVIFFPNWFVFSIYNFAQWARATLMPIAVLSARRPSRPLPAERRLDELFPEGREKYNYAYPEKPGAGFAEAFFLTADKILHALQSFGQTAGIGLLRKGAISRALEWIIKHQDADGVWGGIQPPWIYSLMALYNEGYAHDHSVVAKGLGALDDPRWRVDQGEATWIQASTSPVWDTELTLLALEDCDLSERHEKEVEKALDWLLSQQVLRKGDWSVKCPKLEPGGWAFEYKNYYYPDTDDTAVALIALAPFRNDPKWKDKGIEEAIEKGVEWLIGMQCKNGGWGAFDKDNDKQILTKIPFCDFGEALDPPSVDVTAHIIEAFGKLGIRKDHPVMLRALDYIKTEQEADGAWFGRWGVNYVYGTGAVLPALEAIGEDMTQHYIKKASDWLILHQNEDGGWGESCASYMDVKQMGRGKSTASQTAWALMGLAAVGRAEDERAIADGVQFLIERQKDGAWEEPEYTGTGFPGYGVGQHIKLTDPQLQERLKQGPELSRAFMINYNLYRHYFPMMAMGRVRKMMAGA

SEQ ID NO: 30 - DNA, 2001 bp, Acetobacter syzygii
  source 1..2001: mol_type = genomic DNA
atgggtaactcacttgcgcacaccgttgcggcggcctgtgactggctgatcggcgagcaaaaggctgatggtcactgggtaggcccggtggcatccaatgcaagtatggaagcggagtggtgcttggcgctttggtacctgggtctggaagatcaccctttacggcctcgcctggggaacgccctgttagaaatgcagcgcgaagatgggagttggggcacttactggggcgcgggcaatggtgacattaacgccaccgtggaagcatacgcggctctgcgttccctggggtacccagaggatacaccggcactgtccaaagcgtgcgcgtggattatgcgcatgggcggcctgcgcaatgtccgggtctttacgcgctattggctggcccttattggtgaatggccgtgggaacagactccgaacctaccgccagaagtaatttggttcccaaataagtttgttttctcgatttacaattttgcacaatgggcgcgcgcaacgctggttcccctggccattctgagtgcacgtcgcccatcgcgcccgttacgaccacaggatcgcctggatgcgttgtttccaggcgggcgcgaaaattttgactacgttcttccgaagaaagacggggttgatctgtggagttcattcttccgtactacagatcgtggcttacattggctccagactcggtttctgaaacgaaatacagtcagggaagcagcaatacgtcacatgctggaatggattatcaggcaccaggatgcggacggcggttggggtggcattcagccgccgtgggtgtacggactcatggccttgcacggcgaggattaccagtttcatcaccccgtgatggcaaaagcgctggcagcgctggatgaccctggctggcgccaggatcgtggtgacgcctcatgggtgcaagccaccaattcaccagtgtgggataccatgttagcaattatggcgctgcacgacgccaaagccgaagaacggtataccccgcagatgaataaagcccttgattggctgctcgctcgacaagtacgcgttaaaggtgactggagcatcaaactcccggaagttgaaccgggtggctgggctttcgaatatgccaatgaccgttaccctgacaccgacgacacggcggtggcacttattgcgctttcgtcctgtcgtcatcgggaagaatggaaacagaaaggagtggaagctgccattttgcgcggagtgaactggcttattgcgatgcaaagtacttgcggtggttggggcgcatttgacaaagataacaatcgcagtcttttatctaaaatccctttctgcgactttggcgaagctcttgacccgccgagtgtggacgtcaccgcacatgttcttgaagcgtttgggctcctgggtcttagcccgcaaacgccggctatccagagagggctggcgtacattcgtgctgagcaggaagcatcgggcgcgtggtttggacgctggggtgtcaactacttgtacggaacaggagcggtcctgccagcgctggctgcaattggagaagatatgacgcagccgtacattacccgtgcttgcgattggttggttgcccaccaacaagaggacggtggttggggtgaatcctgtgcctcttatatggacattgctagtatcggtcgaggcactaccaccccgtcccaaacggcgtgggcgctcatgggtctgatcgcggtaaaccgcaaacaagaccatgaagcaatcgcgcgcggttgtcgctacttaatagaccgtcaggaaagcgatggtagctggacggagcaagaatttactggtactggctttccgggatacggtgtaggccaaacaatcaagctggatgacccggccgttgccaagcgtctgcaacagggcgcggaactttctcgggcgtttatgctgcgctatgatctgtaccgacagttctttccgctcatggctctgagtagagcagctaggattattagttttgagcag

SEQ ID NO: 31 - protein, 667 aa, Acetobacter syzygii
MGNSLAHTVAAACDWLIGEQKADGHWVGPVASNASMEAEWCLALWYLGLEDHPLRPRLGNALLEMQREDGSWGTYWGAGNGDINATVEAYAALRSLGYPEDTPALSKACAWIMRMGGLRNVRVFTRYWLALIGEWPWEQTPNLPPEVIWFPNKFVFSIYNFAQWARATLVPLAILSARRPSRPLRPQDRLDALFPGGRENFDYVLPKKDGVDLWSSFFRTTDRGLHWLQTRFLKRNTVREAAIRHMLEWIIRHQDADGGWGGIQPPWVYGLMALHGEDYQFHHPVMAKALAALDDPGWRQDRGDASWVQATNSPVWDTMLAIMALHDAKAEERYTPQMNKALDWLLARQVRVKGDWSIKLPEVEPGGWAFEYANDRYPDTDDTAVALIALSSCRHREEWKQKGVEAAILRGVNWLIAMQSTCGGWGAFDKDNNRSLLSKIPFCDFGEALDPPSVDVTAHVLEAFGLLGLSPQTPAIQRGLAYIRAEQEASGAWFGRWGVNYLYGTGAVLPALAAIGEDMTQPYITRACDWLVAHQQEDGGWGESCASYMDIASIGRGTTTPSQTAWALMGLIAVNRKQDHEAIARGCRYLIDRQESDGSWTEQEFTGTGFPGYGVGQTIKLDDPAVAKRLQQGAELSRAFMLRYDLYRQFFPLMALSRAARIISFEQ

SEQ ID NO: 32 - DNA, 2088 bp, Bradyrhizobium sp.
  source 1..2088: mol_type = genomic DNA; note = STM 3843
atgacactgaatggcaagacattagcggcgactgtctcggcacctgacaactttcagacggccctgcactcgacggttcgcgccgctgccgactggctgattgctcatcagaaaggtgatggccactgggtaggacgtgcggaatcgaacgcctgtatggaagcacagtggtgcttagcactgtggttcatggggctggaagatcatccgctgcgtcctagaatgggccaagcgcttctgcaaacacagcgtgcggacggcagctggcagatttacttcaacgcgccgaatggtgatataaacgcaacggtcgaggcgtacgccgcattacgctcgttagggcaccgtgatgatgaacctgtactgcaaagagcacgccagtggattgaggccaaaggcggactgcgcaacgtccgcgtgtttacccgttattggctcgcgctgctgggtgagtggccgtgggaaaaggtccctaatattccgccagaggttatctggtttccggtgtggtttccgttcagcatctacaattttgcccagtgggcgagggcaactctaatgccaattgcaattttgagtgcccgccgtccaagccgtccgctgccggcggagaaccgcctggatgccctcttcccgaagggtcgcggggcttttgattatgaactcccggtgaaagctgatgcaggcggttgggataaattcttccgcggtgccgacaaagttctgcatgcgctgcaaaacttgggcaattggttgaatctgggcttgtggcgtccggctgctatgtcgcgtgtcctggaatggatgattcgccaccaggatttcgacggtgcctggggcggtattcagccaccgtggatttatggcttgatggcactgtacgtcgaaggataccctatcaaccaccccgttgttgccaaaggcctcgatgcgctaaacgacccgggatggcgtgtggacatcggtgaggcgacttatattcaggcgacgaactctcctgtgtgggataccatcctaaccttactcgcttttgatgatgcgggtgtgattggagattatccagaggcggtcgagaaagccgtgaactgggttctggcacgacaagttcgtgtcccgggcgattggagtgttaaactgcctcatgtgaaacccggcgggtgggctttcgagtacgcgaacaactattatccagacactgacgacaccgcggtcgccttgattgcgctggccccattacgccatgacccgaaatggcaggcgaaaggcattgatgaagcaatccaactgggcgtcgattggttaattggtatgcagagccagtcaggtggctggggcgcctttgataaagacaacaaccaacagatactgacgaaaattcccttctgcgactttggcgaagctctggatccgccatccgtggatgtgacagcccatgtgatagaggcgtttggcaaacttggactgtcaaggaaccatcctaccatggtgcgggcgcttgattacgtgcgccgtgagcaggaaccatccggcccctggttcggtcgctggggcgtaaactatgtgtatggtacgggagcagtccttccggcgctggcggctatcggtgaggacatgagtcagccttacatccagcgcgcatgcgagtggttagtggcgcaacagcaagataacggtgggtggggtgaatcatgtgcgtcttatatggacatttctgctgtgggccgtgggactgccaccgcgagtcaaaccgcctgggccctgatggcgcttttggctgccaaccgtccgggtgaccgagaggcgatcgaaagagggtgcctctggctgatagaacatcagagcgctggtacctgggcggaaccagagttcactggcacggggttcccaggctatggcgtcggccaaactatcaaattaacagatccaagtctccagcaacgcctgatgcaggggccagaactgtctcgtgcttttatgctgcgttatggtatgtattgccactactttccgctgatggcgttaggtcgcacactgcggcctcaggcgcacggcaaagcgcagtccactccagcggcgaacgccgttatt

SEQ ID NO: 33 - protein, 696 aa, Bradyrhizobium sp.
  source 1..696: note = STM 3843
MTLNGKTLAATVSAPDNFQTALHSTVRAAADWLIAHQKGDGHWVGRAESNACMEAQWCLALWFMGLEDHPLRPRMGQALLQTQRADGSWQIYFNAPNGDINATVEAYAALRSLGHRDDEPVLQRARQWIEAKGGLRNVRVFTRYWLALLGEWPWEKVPNIPPEVIWFPVWFPFSIYNFAQWARATLMPIAILSARRPSRPLPAENRLDALFPKGRGAFDYELPVKADAGGWDKFFRGADKVLHALQNLGNWLNLGLWRPAAMSRVLEWMIRHQDFDGAWGGIQPPWIYGLMALYVEGYPINHPVVAKGLDALNDPGWRVDIGEATYIQATNSPVWDTILTLLAFDDAGVIGDYPEAVEKAVNWVLARQVRVPGDWSVKLPHVKPGGWAFEYANNYYPDTDDTAVALIALAPLRHDPKWQAKGIDEAIQLGVDWLIGMQSQSGGWGAFDKDNNQQILTKIPFCDFGEALDPPSVDVTAHVIEAFGKLGLSRNHPTMVRALDYVRREQEPSGPWFGRWGVNYVYGTGAVLPALAAIGEDMSQPYIQRACEWLVAQQQDNGGWGESCASYMDISAVGRGTATASQTAWALMALLAANRPGDREAIERGCLWLIEHQSAGTWAEPEFTGTGFPGYGVGQTIKLTDPSLQQRLMQGPELSRAFMLRYGMYCHYFPLMALGRTLRPQAHGKAQSTPAANAVI

SEQ ID NO: 34 - DNA, 2025 bp, Gluconobacter frateurii
  source 1..2025: mol_type = genomic DNA; note = NBRC 101659
atgagcgcaccgattctcaaaggcatgagcaacagcttagctcatacggtgagttgtgcgtgtgattggctcattggtcaacagaagggtgatggccactgggtcggtagcgtggagagcaatgcttctatggaagccgaatggtgcctggcactctggttcttgggtctggaagatcatccgttgcgtccgcgtttggggaatgcgctgctggaaatgcagcgtgatgacggctcctggggcgtgtacctgggcgcgcgtagcggcgacatcaatgctaccgtagaggcctatgcggcgttacgttcactcgggtactccgcaaactctccagtcctgctgaaggcttcgatgtggattgctgaaaagggcggattaaagaatattcgggtattcacccgctactggttagccctgatcggcgaatggccttgggagaaaacaccaaatttgccgccggaagtaatctggttcccgaataactttgtctttagcatctataattttgcgcaatgggcgcgagcgactctggccccgttagccatcttatccgcccgtcgtccaagccgtccattgcgccctcaggatagactggatgcgctgttcccagaaggccgcgagaaattcgattacacgcttccgaagaaggatcgcgttgacttatggtcgtccttctttcgaaccaccgataaaggtttgcactggctgcaaagccgctttctgaaacgtaacaccgttcgcgaagctgccattcgccacatgctcgaatggattattcgtcaccaagacgcagatggcgggtggggcggtatccagccgccgtgggtctacggcctcatggcgttgcatggagaaggctatcctttccatcatcctgttctggccaaagcgttagctgccctggacgacccgggttggcgctatgatcgcggcgaagcatcatggattcaggccaccaactccccggtgtgggatacgatgctggcacttatggcactgtacgatgcaaacgcacaagaacgttttacgccggaaatggacaaagctctggagtggctgctcaaccgccaggtgcgcgtgaaaggcgactggtccataaaactccccgatgttgagccgggcggttggagctttgaatatgcgaatgatcgttatccagataccgatgatactgctgttgcgttgatcgcactgtctttctgtcgccacaaagaagaatggaaacgcaaaggcgtcgatgaagcgattgatcgcgcagtcaactggctgatcgcaatgcaatcatcgtgcggtggttggggcgcgttcgacaaagataacaacaaatccctgcttagcaagatcccgttctgcgacttcggtgaggctttggatccgccatcggttgatgtcacggcccatatcttagaggcatttggtttgcttggtctgtcgcgcgacttgcccgcggtccagaaagccttagcctacgttcgttctgaacaagatccgcaggggccttggtttggacgttggggcgtcaactatctgtatggtacaggcgcggtcctgccggcgctggccgccattggcgaagatatgactcaaccctacattacgaacgcgtgcgactggttaatatcttgtcagcaggatgatggcggctggggtgaaagttgcgccagttatatggacatttcgtccattgggcgcggctcgacgactgccagccagacagcgtgggcgcttatgggccttatcgctgtgggtcgcccgcaggattacgaagccattgcgaagggctgccgctttctgatcgatcggcaagaggcggacggcagttggaccgagcaggagtttacgggcacaggttttcctggttatggcgtaggacagaccattaagcttgatgatccggccttgagtaaacgtttgatgcagggtgcggaactgagccgcgcttttatgttgcgttacgacatgtatcgtcagtactttccgattatggcgctggcccgcgcgaatcgcttgctctcccaggatgtt

SEQ ID NO: 35 - protein, 675 aa, Gluconobacter frateurii
  source 1..675: note = NBRC 101659
MSAPILKGMSNSLAHTVSCACDWLIGQQKGDGHWVGSVESNASMEAEWCLALWFLGLEDHPLRPRLGNALLEMQRDDGSWGVYLGARSGDINATVEAYAALRSLGYSANSPVLLKASMWIAEKGGLKNIRVFTRYWLALIGEWPWEKTPNLPPEVIWFPNNFVFSIYNFAQWARATLAPLAILSARRPSRPLRPQDRLDALFPEGREKFDYTLPKKDRVDLWSSFFRTTDKGLHWLQSRFLKRNTVREAAIRHMLEWIIRHQDADGGWGGIQPPWVYGLMALHGEGYPFHHPVLAKALAALDDPGWRYDRGEASWIQATNSPVWDTMLALMALYDANAQERFTPEMDKALEWLLNRQVRVKGDWSIKLPDVEPGGWSFEYANDRYPDTDDTAVALIALSFCRHKEEWKRKGVDEAIDRAVNWLIAMQSSCGGWGAFDKDNNKSLLSKIPFCDFGEALDPPSVDVTAHILEAFGLLGLSRDLPAVQKALAYVRSEQDPQGPWFGRWGVNYLYGTGAVLPALAAIGEDMTQPYITNACDWLISCQQDDGGWGESCASYMDISSIGRGSTTASQTAWALMGLIAVGRPQDYEAIAKGCRFLIDRQEADGSWTEQEFTGTGFPGYGVGQTIKLDDPALSKRLMQGAELSRAFMLRYDMYRQYFPIMALARANRLLSQDV

SEQ ID NO: 36 - DNA, 2010 bp, Acetobacter ghanensis
  source 1..2010: mol_type = genomic DNA
atggcaacccttcgcgaagccgcgattaaacacatgctggaatggattattcgccatcaggacgcagacggcggttggggcggcattcagccgccatgggtgtacggcttaatggcactacatggtgaagattatcagttccaccatccggtgatggctaaggctttagccgccttagacgatcctggctggcgtcaagatcgtggggatgccagctgggtgcaggccacgaacagtccagtatgggatacgatgttagcactgatggcgctccacgacgcgggcgctgaagagcgctatacccctgaaatggataaagcgctggattggctgttacagcgccaggttcgggtaacaggtgactggtcaatcaaactgccgggagtcgaacctggcggttgggcattcgaatatgcgaatgaccgttatcccgataccgacgataccgcggttgcgctcattgcactgagcgcgtgtcgccaccgggaagaatggaagaagaaaggggtcgaagctgcgatcacacgcggagtaaattggctcattgccatgcagtcaacgtgcggcggctggggcgcatttgataaagataacaatcgttcgttactgtcgaagattccattttgtgactttggcgaagctctggacccacccagcgtcgatgtcacggcacatgtgcttgaagcctttggtctgctgggtctgccccgcgaaaccccttccattcagcgcggccttgcttacatcagagcggaacaggagccgtctggagcttggtttggccgatggggcgttaattacctgtatgggaccggggcagtcctaccggccctggcggcaatcggtgaggatatgacccagccgtacatcactcgtgcgtgtgattggctcgtcgctcatcagcaggaaaacggtggctggggtgaatcgtgtgcgtcatatatggacgtcgcgtcgattggtcatggaactccgaccgcctctcagaccgcatgggccctgatgggactgattgcggtgaatcgaccgcaggatcacgaggcaatcgcacgcggctgtcgttttctgattgaccgtcaggaagaagatggaagttggaccgaagaggaatttaccggtaccggctttccaggctacggtgtcggccagactatcaaactggacgatccagccgtcgctaaacgtctgcaacagggtgcggaactttcgcgtgcgttcatgctacgctacgacctgtatcgtcagttcttcccgctgatggccctgagccgtgcggcgcgtatcattggtggcggtagcggtggcggtagctcgcttgcgcacacggtcgcagcggcgtgtgattggctgatcggggagcagaaagccgatggacattgggtgggtccggtagcgtccaatgcatcaatggaagcggaatggtgcctggctttatggtacctgggtctggaagatcacccgctgcgcccacgtttaggcaacgccctgttgcagatgcagcgagaggacggttcttggggcatttactggggcgccgggaacggtgacattaatgcaacggtagaagcttacgcggcactacgttcgttaggctacccggctgacacgccagctttaagcaaagcgtgtgcgtggattatgcgtatgggcggcctgcgcaatatccgcgtgttcactcgttactggctcgcgctgattggcgaatggccgtgggaacagacgccgaacctgccgccggagatcatctggtttccgaataaattcgttttcagcatttacaactttgcacagtgggcccgtgcaacattagtgcctctggcaattctgagtgcaagacgcccgtcacgtcctctccgcccgcaggatcgtctggatgcgttgttcccgggcggtcgggaaaatttcgactatgtgctgcctaaacgcgatggcatggacctgtggtcctcattctttcgcaccacggaccgcggcctgcactggctgcaatcaaaatttctgggcgcgtaa

SEQ ID NO: 37 - protein, 669 aa, Acetobacter ghanensis
MATLREAAIKHMLEWIIRHQDADGGWGGIQPPWVYGLMALHGEDYQFHHPVMAKALAALDDPGWRQDRGDASWVQATNSPVWDTMLALMALHDAGAEERYTPEMDKALDWLLQRQVRVTGDWSIKLPGVEPGGWAFEYANDRYPDTDDTAVALIALSACRHREEWKKKGVEAAITRGVNWLIAMQSTCGGWGAFDKDNNRSLLSKIPFCDFGEALDPPSVDVTAHVLEAFGLLGLPRETPSIQRGLAYIRAEQEPSGAWFGRWGVNYLYGTGAVLPALAAIGEDMTQPYITRACDWLVAHQQENGGWGESCASYMDVASIGHGTPTASQTAWALMGLIAVNRPQDHEAIARGCRFLIDRQEEDGSWTEEEFTGTGFPGYGVGQTIKLDDPAVAKRLQQGAELSRAFMLRYDLYRQFFPLMALSRAARIIGGGSGGGSSLAHTVAAACDWLIGEQKADGHWVGPVASNASMEAEWCLALWYLGLEDHPLRPRLGNALLQMQREDGSWGIYWGAGNGDINATVEAYAALRSLGYPADTPALSKACAWIMRMGGLRNIRVFTRYWLALIGEWPWEQTPNLPPEIIWFPNKFVFSIYNFAQWARATLVPLAILSARRPSRPLRPQDRLDALFPGGRENFDYVLPKRDGMDLWSSFFRTTDRGLHWLQSKFLGA

SEQ ID NO: 38 - DNA, 2013 bp, Acetobacter ghanensis
  source 1..2013: mol_type = genomic DNA
atggcagaatggaagaagaaaggggtcgaagctgcgatcacacgcggagtaaattggctcattgccatgcagtcaacgtgcggcggctggggcgcatttgataaagataacaatcgttcgttactgtcgaagattccattttgtgactttggcgaagctctggacccacccagcgtcgatgtcacggcacatgtgcttgaagcctttggtctgctgggtctgccccgcgaaaccccttccattcagcgcggccttgcttacatcagagcggaacaggagccgtctggagcttggtttggccgatggggcgttaattacctgtatgggaccggggcagtcctaccggccctggcggcaatcggtgaggatatgacccagccgtacatcactcgtgcgtgtgattggctcgtcgctcatcagcaggaaaacggtggctggggtgaatcgtgtgcgtcatatatggacgtcgcgtcgattggtcatggaactccgaccgcctctcagaccgcatgggccctgatgggactgattgcggtgaatcgaccgcaggatcacgaggcaatcgcacgcggctgtcgttttctgattgaccgtcaggaagaagatggaagttggaccgaagaggaatttaccggtaccggctttccaggctacggtgtcggccagactatcaaactggacgatccagccgtcgctaaacgtctgcaacagggtgcggaactttcgcgtgcgttcatgctacgctacgacctgtatcgtcagttcttcccgctgatggccctgagccgtgcggcgcgtatcattggtggcggtagcggtggcggtagctcgcttgcgcacacggtcgcagcggcgtgtgattggctgatcggggagcagaaagccgatggacattgggtgggtccggtagcgtccaatgcatcaatggaagcggaatggtgcctggctttatggtacctgggtctggaagatcacccgctgcgcccacgtttaggcaacgccctgttgcagatgcagcgagaggacggttcttggggcatttactggggcgccgggaacggtgacattaatgcaacggtagaagcttacgcggcactacgttcgttaggctacccggctgacacgccagctttaagcaaagcgtgtgcgtggattatgcgtatgggcggcctgcgcaatatccgcgtgttcactcgttactggctcgcgctgattggcgaatggccgtgggaacagacgccgaacctgccgccggagatcatctggtttccgaataaattcgttttcagcatttacaactttgcacagtgggcccgtgcaacattagtgcctctggcaattctgagtgcaagacgcccgtcacgtcctctccgcccgcaggatcgtctggatgcgttgttcccgggcggtcgggaaaatttcgactatgtgctgcctaaacgcgatggcatggacctgtggtcctcattctttcgcaccacggaccgcggcctgcactggctgcaatcaaaatttctgaaacgcaacacccttcgcgaagccgcgattaaacacatgctggaatggattattcgccatcaggacgcagacggcggttggggcggcattcagccgccatgggtgtacggcttaatggcactacatggtgaagattatcagttccaccatccggtgatggctaaggctttagccgccttagacgatcctggctggcgtcaagatcgtggggatgccagctgggtgcaggccacgaacagtccagtatgggatacgatgttagcactgatggcgctccacgacgcgggcgctgaagagcgctatacccctgaaatggataaagcgctggattggctgttacagcgccaggttcgggtaacaggtgactggtcaatcaaactgccgggagtcgaacctggcggttgggcattcgaatatgcgaatgaccgttatcccgataccgacgataccgcggttgcgctcattgcactgagcgcgtgtcgccacggcgcgtaa

SEQ ID NO: 39 - protein, 670 aa, Acetobacter ghanensis
MAEWKKKGVEAAITRGVNWLIAMQSTCGGWGAFDKDNNRSLLSKIPFCDFGEALDPPSVDVTAHVLEAFGLLGLPRETPSIQRGLAYIRAEQEPSGAWFGRWGVNYLYGTGAVLPALAAIGEDMTQPYITRACDWLVAHQQENGGWGESCASYMDVASIGHGTPTASQTAWALMGLIAVNRPQDHEAIARGCRFLIDRQEEDGSWTEEEFTGTGFPGYGVGQTIKLDDPAVAKRLQQGAELSRAFMLRYDLYRQFFPLMALSRAARIIGGGSGGGSSLAHTVAAACDWLIGEQKADGHWVGPVASNASMEAEWCLALWYLGLEDHPLRPRLGNALLQMQREDGSWGIYWGAGNGDINATVEAYAALRSLGYPADTPALSKACAWIMRMGGLRNIRVFTRYWLALIGEWPWEQTPNLPPEIIWFPNKFVFSIYNFAQWARATLVPLAILSARRPSRPLRPQDRLDALFPGGRENFDYVLPKRDGMDLWSSFFRTTDRGLHWLQSKFLKRNTLREAAIKHMLEWIIRHQDADGGWGGIQPPWVYGLMALHGEDYQFHHPVMAKALAALDDPGWRQDRGDASWVQATNSPVWDTMLALMALHDAGAEERYTPEMDKALDWLLQRQVRVTGDWSIKLPGVEPGGWAFEYANDRYPDTDDTAVALIALSACRHGA

SEQ ID NO: 40 - protein, 6 aa, synthetic construct
GGGGGG

SEQ ID NO: 41 - protein, 10 aa, synthetic construct
GGGGGGGGGG

SEQ ID NO: 42 - protein, 12 aa, synthetic construct
GGGGGGGGGGGG

SEQ ID NO: 43 - protein, 11 aa, synthetic construct
GGSGGTGGSGG

SEQ ID NO: 44 - protein, 12 aa, synthetic construct
GGSGREEKGSGG

SEQ ID NO: 45 - protein, 9 aa, synthetic construct
GGGSTSGGG

SEQ ID NO: 46 - protein, 10 aa, synthetic construct
AVNRPQDHEA

SEQ ID NO: 47 - protein, 9 aa, synthetic construct
AIGEDMTQP

SEQ ID NO: 48 - protein, 8 aa, synthetic construct
LGLPRETP

SEQ ID NO: 49 - protein, 9 aa, synthetic construct
AGAEERYTP

SEQ ID NO: 50 - protein, 9 aa, synthetic construct
ACRHREEWK

SEQ ID NO: 51 - protein, 8 aa, synthetic construct
GGGSGGGS

SEQ ID NO: 52 - protein, 12 aa, synthetic construct
GGGSGGGSGGGS

SEQ ID NO: 53 - protein, 12 aa, synthetic construct
GGGSGGGSGGGP